(19) United States
(12) Patent Application Publication
(10) Pub. No.: US 2025/0242049 A1
(43) Pub. Date: Jul. 31, 2025

(54) LIPID COMPOSITION AND METHOD OF DELIVERING THERAPEUTIC AGENT

(71) Applicants: FUJIFILM Corporation, Tokyo (JP); Massachusetts Institute of Technology, Cambridge, MA (US)

(72) Inventors: Sho TOYONAGA, Cambridge, MA (US); Daniel Griffith ANDERSON, Framingham, MA (US); Theresa Marie RAIMONDO, Greenville, RI (US); Dennis Zheng SHI, Somerville, MA (US)

(73) Assignees: FUJIFILM Corporation, Tokyo (JP); Massachusetts Institute of Technology, Cambridge, MA (US)

(21) Appl. No.: 19/181,743

(22) Filed: Apr. 17, 2025

Related U.S. Application Data
(63) Continuation of application No. PCT/JP2023/037715, filed on Oct. 18, 2023.
(60) Provisional application No. 63/417,563, filed on Oct. 19, 2022.

(51) Int. Cl. A61K 47/69; A61K 31/167; A61K 31/7105; A61K 31/711; A61K 45/06; A61K 47/54; A61K 47/60; A61K 47/68

(52) U.S. Cl. CPC A61K 47/6929; A61K 31/167; A61K 31/7105; A61K 31/711; A61K 45/06; A61K 47/543; A61K 47/545; A61K 47/60; A61K 47/6849

(57) ABSTRACT

It is an object of the present invention to provide a lipid composition capable of delivering a nucleic acid such as RNA to a hematopoietic stem/progenitor cell or mesenchymal stem cells, and a method of delivering a therapeutic agent to a cell using the lipid composition. The present invention provides a lipid composition comprising (A) a therapeutic agent and (B) a lipid nanoparticle conjugated to a targeting molecule, wherein the lipid nanoparticle comprises an ionizable lipid, and the targeting molecule specifically binds to a marker of hematopoietic stem/progenitor cells or mesenchymal stem cells.

### CROSS-REFERENCE TO RELATED APPLICATIONS

This application is a Continuation of PCT International Application No. PCT/JP2023/037715 filed on Oct. 18, 2023, which claims priority under 35 U.S.C § 119(a) to U.S. Provisional Patent Application No. 63/417,563 filed on Oct. 19, 2022. Each of the above application(s) is hereby expressly incorporated by reference, in its entirety, into the present application.

### TECHNICAL FIELD

The present invention relates to a lipid composition comprising a therapeutic agent and a lipid nanoparticle (LNP), and a method of delivering a therapeutic agent to a cell using the lipid composition.

### BACKGROUND OF THE INVENTION

Hematopoietic stem/progenitor cells (HSPCs) and mesenchymal stem cells (MSCs) are key targets for gene therapy. Current gene therapy protocols include harvesting HSPCs or MSCs from donors/patients, in vitro culturing, transduction by retroviral vector, and reimplantation into bone marrow conditioned patients. In addition to technical complexity and manufacturing cost, disadvantages of this approach include the need for cultures in the presence of multiple cytokines that can affect the pluripotency and engraftment of HSPCs. Moreover, the need for myeloablative procedures such as total body irradiation (TBI) or fatal chemotherapy including busulfan/cyclophosphamide (BU/CY) in patients with non-malignant diseases creates additional risks.

Also, in terms of allogeneic transplantation, donor HSC or MSC must escape immune rejection by the recipient. Recognition of HLA incompatibility by the immune system is a major barrier to allogeneic hematopoietic stem cell transplantation. Thus, sibling donors with identical HLA genotypes are the gold standard for transplant purposes, but only 30% of patients have such a donor. For the remaining 70% of patients, alternative sources of stem cells are matched unrelated adult volunteer donors, semi-matched donors or umbilical cord blood units.

On the other hand, CD117 is known to be expressed in hematopoietic stem cells. Non-Patent Document 1 describes the utility of anti CD117 antibody-modified nanoparticles and the combination of recruitment of hematopoietic stem/progenitor cells with anti-antibody-modified nanoparticles. Patent Document 1 describes charged lipids/polymers and anti CD117 antibodies. Patent Document 2 describes liposomes coated with an anti CD117 antibody. However, Non-Patent Document 1 and Patent Documents 1 and 2 do not describe a combination of an ionizable lipid and an anti CD117 antibody.

### CITATION LIST

### Patent Literature

Patent Document 1: WO2019/213308
Patent Document 2: WO2015/153805

### Non Patent Literature

Non-Patent Document 1: Paula Cannon et al., HUMAN GENE THERAPY, VOLUME 32, NUMBERS 1 and 2 (pages 31-43) DOI: 10.1089/hum.2020.263

### SUMMARY OF INVENTION

### Object to be Solved by the Invention

The present invention addresses this need by providing a lipid composition capable of delivering a nucleic acid such as RNA to a hematopoietic stem/progenitor cell, and a method of delivering a therapeutic agent to a cell using the lipid composition.

### Means for Solving the Object

As a result of extensive studies to solve the above object, the present inventors have found that a therapeutic agent can be efficiently delivered to a hematopoietic stem/progenitor cell by administering a lipid nanoparticle to which a targeting molecule that specifically binds to a marker of a hematopoietic stem/progenitor cell is bound wherein a therapeutic agent is encapsulated. The present invention has been completed based on the above findings.

According to the present invention, there is provided the following invention.

<1> A lipid composition comprising (A) a therapeutic agent and (B) a lipid nanoparticle conjugated to a targeting molecule,
wherein the lipid nanoparticle comprises an ionizable lipid, and
the targeting molecule specifically binds to a marker of hematopoietic stem/progenitor cells or mesenchymal stem cells.
   <2> The lipid composition of <1>, wherein the lipid nanoparticle comprises a PEG-lipid conjugated to the targeting molecule.
   <3> The lipid composition of <1>, wherein the ionizable lipid has at least one ionizable amino group and at least one biodegradable group, and wherein the biodegradable group is represented by —O(CO)O—, —O(CO)—, —(CO)O— or S—S.
   <4> The lipid composition of <1>, wherein the ionizable lipid is a compound represented by formula (4):

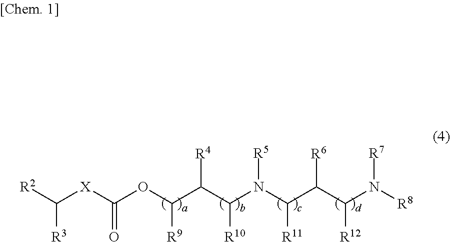

wherein X represents NR¹— or —O—,
R¹ represents a hydrogen atom, a hydrocarbon group having 6 to 24 carbon atoms, or a group represented by R²¹-L¹-R²²—, R²¹ represents a hydrocarbon group having 1 to 24 carbon atoms, and L¹ represents —O(CO)O—, —O(CO)—, —(CO)O—, —O—, or

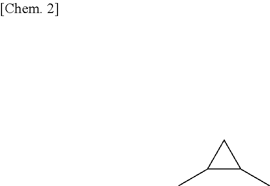

R²² is a divalent linking group and represents a hydrocarbon linking group having 1 to 18 carbon atoms,
R² and R³ each independently represent a hydrogen atom, a hydrocarbon group having 3 to 24 carbon atoms, or a group represented by R³¹-L²-R³²—, R³¹ represents a hydrocarbon group having 1 to 24 carbon atoms, and L² represents —O(CO)O—, —O(CO)—, —(CO)O—, —O—, or

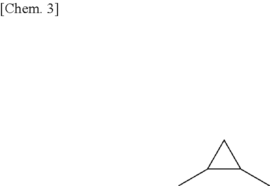

R³² is a divalent linking group and represents a hydrocarbon linking group having 1 to 18 carbon atoms,
each of R⁴, R⁵, R⁶, R⁷, R⁸, R⁹, R¹⁰, R¹¹, and R¹² independently represents a hydrogen atom or an optionally substituted alkyl group having 1-18 carbon atoms,
any one or more sets of R⁴ and R⁵, R¹⁰ and R⁵, R⁵ and R¹², R⁴ and R⁶, R⁵ and R⁶, R⁶ and R⁷, R⁶ and R¹⁰, R¹² and R⁷, and R⁷ and R⁸, may be linked together to form a 4- to 7-membered ring which may contain O atom,
a substituents on the optionally substituted alkyl group having 1-18 carbon atoms represents a hydroxyl groups, a carboxyl groups, an amino groups represented by NR⁴⁵R⁴⁶, a substituted or unsubstituted aryl group, a substituted or unsubstituted heteroaryl group, or a group represented by —O(CO)O—R⁴¹, —O(CO)—R⁴², —(CO)O—R⁴³, or —O—R⁴⁴, wherein R⁴¹, R⁴², R⁴³, R⁴⁴, R⁴⁵ and R⁴⁶ each independently represents a hydrocarbon group having 1 to 18 carbon atoms,
a substituents on the substituted or unsubstituted aryl group and the substituted or unsubstituted heteroaryl group represents an alkyl groups having 1 to 18 carbon atoms, a hydroxyl groups, a carboxyl groups, an amino groups represented by —NR⁴⁵R⁴⁶, or a group represented by —O(CO)O—R⁴¹, —O(CO)—R⁴², —(CO)O—R⁴³, or —O—R⁴⁴, and R⁴¹, R⁴², R⁴³, R⁴⁴, R⁴⁵ and R⁴⁶ each independently represents a hydrocarbon group having 1 to 18 carbon atoms,
a, b, c, and d each independently represents an integer from 0 to 3, wherein a+b is 1 or more, and c+d is 1 or more.
   <5> The lipid composition of <1>, wherein the ionizable lipid is a compound represented by formula (1):

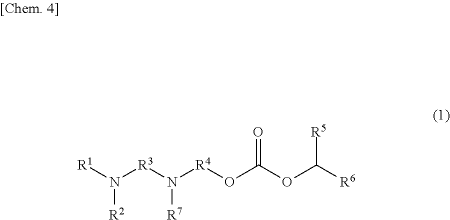

In the formula,
R¹ and R² each independently represent a hydrocarbon group having 1 to 18 carbon atoms, and R³ represents a hydrocarbon group having 2 to 8 carbon atoms, where the hydrocarbon groups represented by R¹, R², and R³ may be substituted with one or more substituents selected from —OH, COOH, —NR⁵¹R⁵², —OC(O)O—R⁵³, —C(O)O-—R⁵⁴, —OC(O)—R⁵⁵, and —O—R⁵⁶,
R⁴ represents a hydrocarbon group having 1 to 8 carbon atoms,
R⁵ and R⁶ each independently represent a hydrocarbon group having 1 to 8 carbon atoms or —R⁸-L¹-R⁹, excluding a case that both R⁵ and R⁶ are hydrocarbon groups having 1 to 8 carbon atoms,
R⁷ represents —R¹⁰-L²-R¹¹-L³-R¹²,
R⁵¹ and R⁵² each independently represent a hydrocarbon group having 1 to 8 carbon atoms,
R⁵³, R⁵⁴, R⁵⁵, and R⁵⁶ each independently represent a hydrocarbon group having 1 to 24 carbon atoms,
the hydrocarbon groups represented by R⁵³, R⁵⁴, R⁵⁵, and R⁵⁶ may be substituted with an aryl group having 6 to 20 carbon atoms or —S—R⁵⁸,
the above-described aryl group having 6 to 20 carbon atoms may be substituted with —OH, COOH, —NR⁵¹R⁵², —OC(O)O—R⁵³, —C(O)O—R⁵⁴, —OC(O)—R⁵⁵, —O—R⁵⁶, or -(hydrocarbon group having 1 to 12 carbon atoms)-R⁵⁷,
R⁵⁸ represents a hydrocarbon group having 1 to 12 carbon atoms, and
R⁵⁷ represents —OH, COOH, —NR⁶¹R⁶², —OC(O)O—R⁶³, —C(O)O—R⁶⁴, —OC(O)—R⁶⁵, or —O—R⁶⁶.
R⁶¹ and R⁶² each independently represent a hydrocarbon group having 1 to 8 carbon atoms,
R⁶³, R⁶⁴, R⁶⁵, and R⁶⁶ each independently represent a hydrocarbon group having 1 to 24 carbon atoms,
the hydrocarbon groups represented by R⁶³, R⁶⁴, R⁶⁵, and R⁶⁶ may be substituted with an aryl group having 6 to 20 carbon atoms or —S—R⁶⁸,
the above-described aryl group having 6 to 20 carbon atoms may be substituted with —OH, COOH, —NR⁶¹R⁶², —OC(O)O—R⁶³, —C(O)O—R⁶⁴, —OC(O)—R⁶⁵, —O—R⁶⁶, or -(hydrocarbon group having 1 to 12 carbon atoms)-R⁶⁷,
R⁶⁸ represents a hydrocarbon group having 1 to 12 carbon atoms, and
L¹, L², and L³ each independently represent —OC(O)O—, —C(O)O—, —OC(O)—, or —O—.
R⁸ represents a hydrocarbon group having 1 to 12 carbon atoms,
R⁹ represents a hydrocarbon group having 1 to 24 carbon atoms,
R¹⁰ represents a hydrocarbon group having 1 to 8 carbon atoms,
R¹¹ represents a hydrocarbon group having 1 to 24 carbon atoms,
R¹² represents a hydrocarbon group having 1 to 24 carbon atoms,
the hydrocarbon groups represented by R⁹ and R¹² may be substituted with an aryl group, —OC(O)O—R⁵³, —C(O)O—R⁵⁴, —OC(O)—R⁵⁵, or —S—R⁵⁸, where definitions of R⁵³, R⁵⁴, R⁵⁵, and R⁵⁸ are as described above, and
the hydrocarbon group represented by R¹¹ may be substituted with —OC(O)O—R⁵³, —C(O)O—R⁵⁴, or —OC(O)—R⁵⁵, where the definitions of R⁵³, R⁵⁴, and R⁵⁵ are as described above.
   <6> The lipid composition of <1>, wherein the ionizable lipid is a compound represented by the following formula (5):

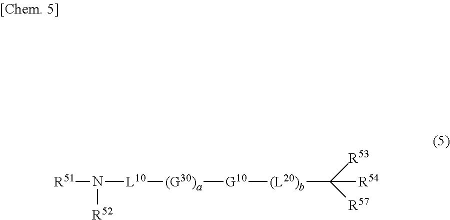

wherein R⁵¹ and R⁵² each independently represent a hydrocarbon group having 1 to 21 carbon atoms which may have a substituent A,
the substituent A represents a hydroxyl group, or a group represented by -G²⁰-CH(R⁵⁵)(R⁵⁶), —N(R⁵⁸)(R⁵⁹) or -G²⁰-R⁶⁰,
G²⁰ represents —O(CO)—, or —(CO)O—,
R⁵⁵ and R⁵⁶ each independently represent a hydrogen atom or a hydrocarbon group having 1 to 18 carbon atoms,
R⁵⁸ and R⁵⁹ each independently represent a hydrogen atom or a cyclic hydrocarbon group having 3 to 6 carbon atoms which may have a substituent B,
the substituent B is —N(R⁶¹)(R⁶²),
R⁶¹ and R⁶² each independently represent a hydrogen atom or a hydrocarbon group having 1 to 6 carbon atoms,
R⁶⁰ represents a hydrocarbon group having 1 to 18 carbon atoms,
L¹⁰ represents a hydrocarbon group having 1 to 18 carbon atoms,
G³⁰ indicates —S—(CO)—NR⁶⁴,
R⁶⁴ represents a group represented by -L³⁰-G²⁰-CH(R⁵⁵)(R⁵⁶),
a represents 0 or 1,
L³⁰ represents a single bond or a hydrocarbon group having 1 to 18 carbon atoms,
G¹⁰ represents —O(CO)—, —(CO)O—, —O(CO)O—, or —N(C(O)R⁶³)—,
R⁶³ represents a hydrocarbon group having 1 to 18 carbon atoms,
L²⁰ represents a hydrocarbon group having 1 to 6 carbon atoms,
b represents 0 or 1,
R⁵³, R⁵⁴ and R⁵⁷ each independently represent a hydrogen atom or a hydrocarbon group having 1 to 21 carbon atoms which may have a substituent C,
the substituent C represents a group represented by —(CO)OR⁶⁵ or —O(CO)—R⁶⁵,
R⁶⁵ represents a hydrocarbon group having 1 to 18 carbon atoms or a group represented by -L⁴⁰-CH(R⁶⁶)(R⁶⁷),
L⁴⁰ represents a hydrocarbon group having 1 to 6 carbon atoms,
R⁶⁶ and R⁶⁷ represent a hydrocarbon group having 1 to 10 carbon atoms or an alkoxy group.
   <7> The lipid composition of <1>, wherein the ionizable lipid is at least one which is selected from the compounds represented by the following formulas.

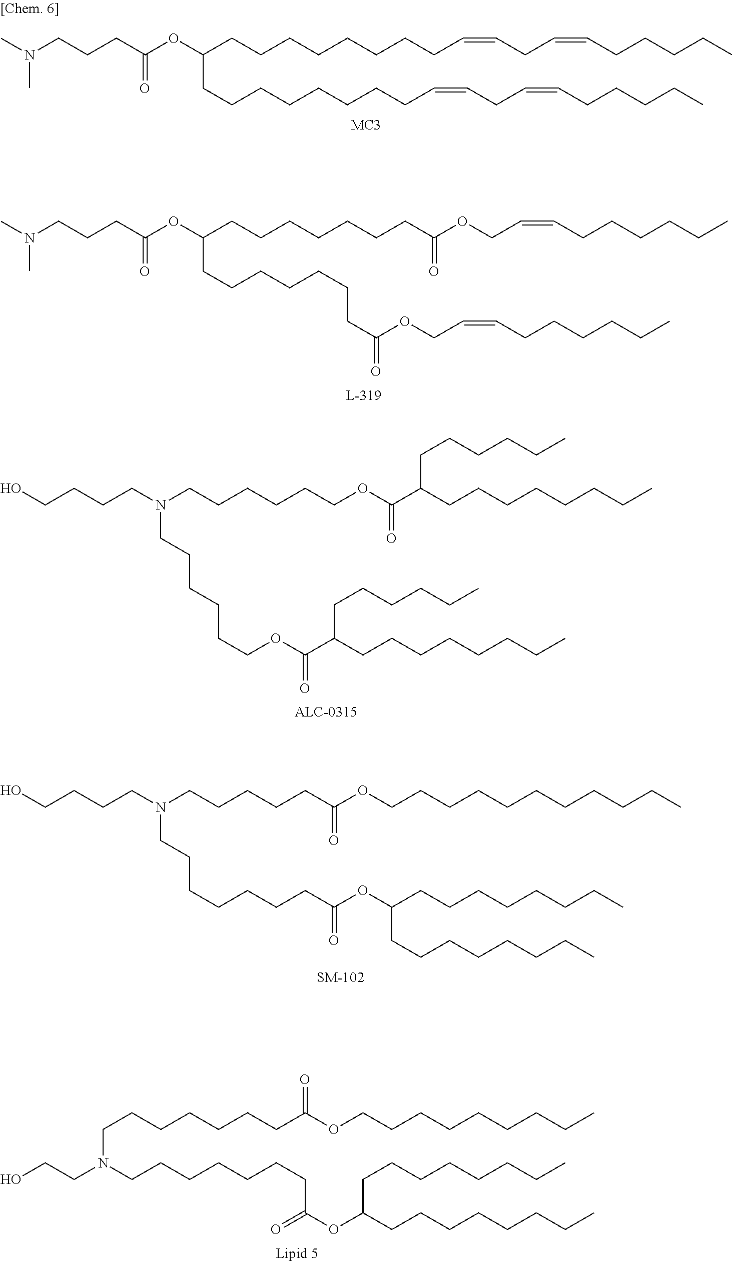

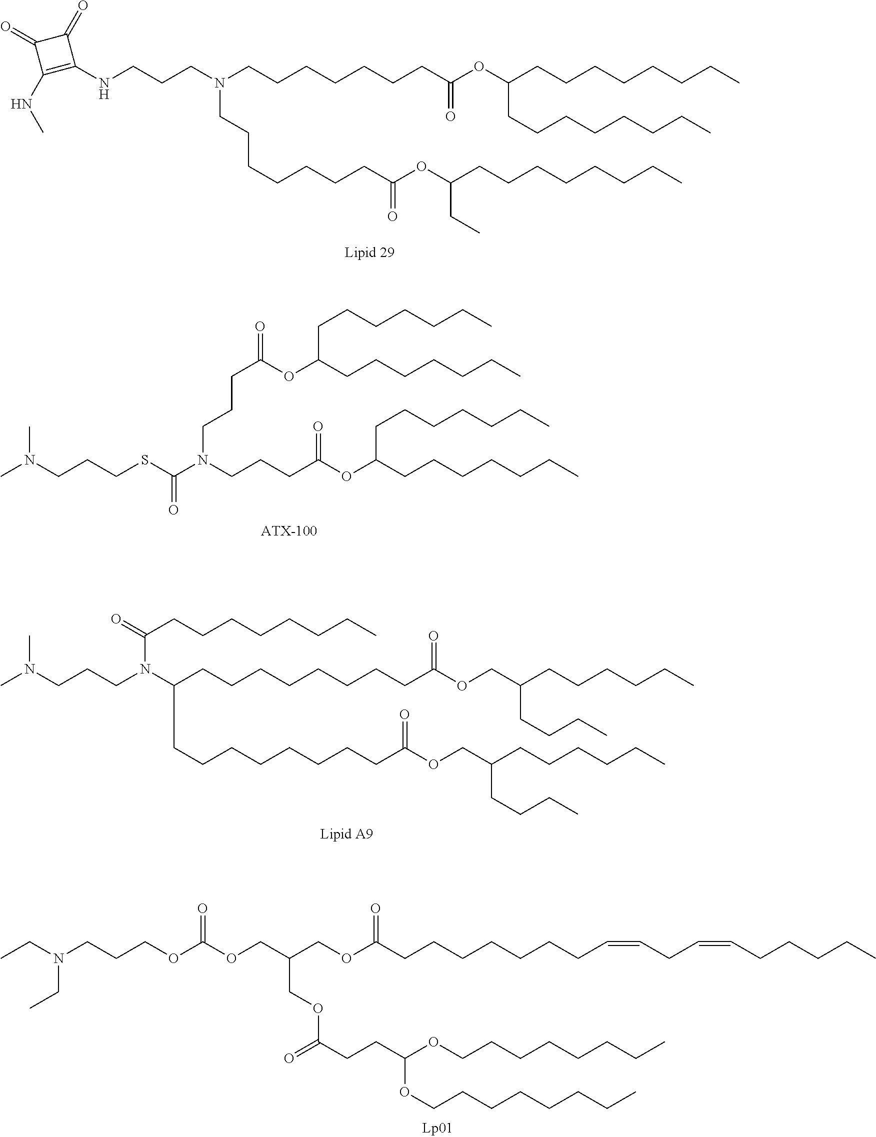

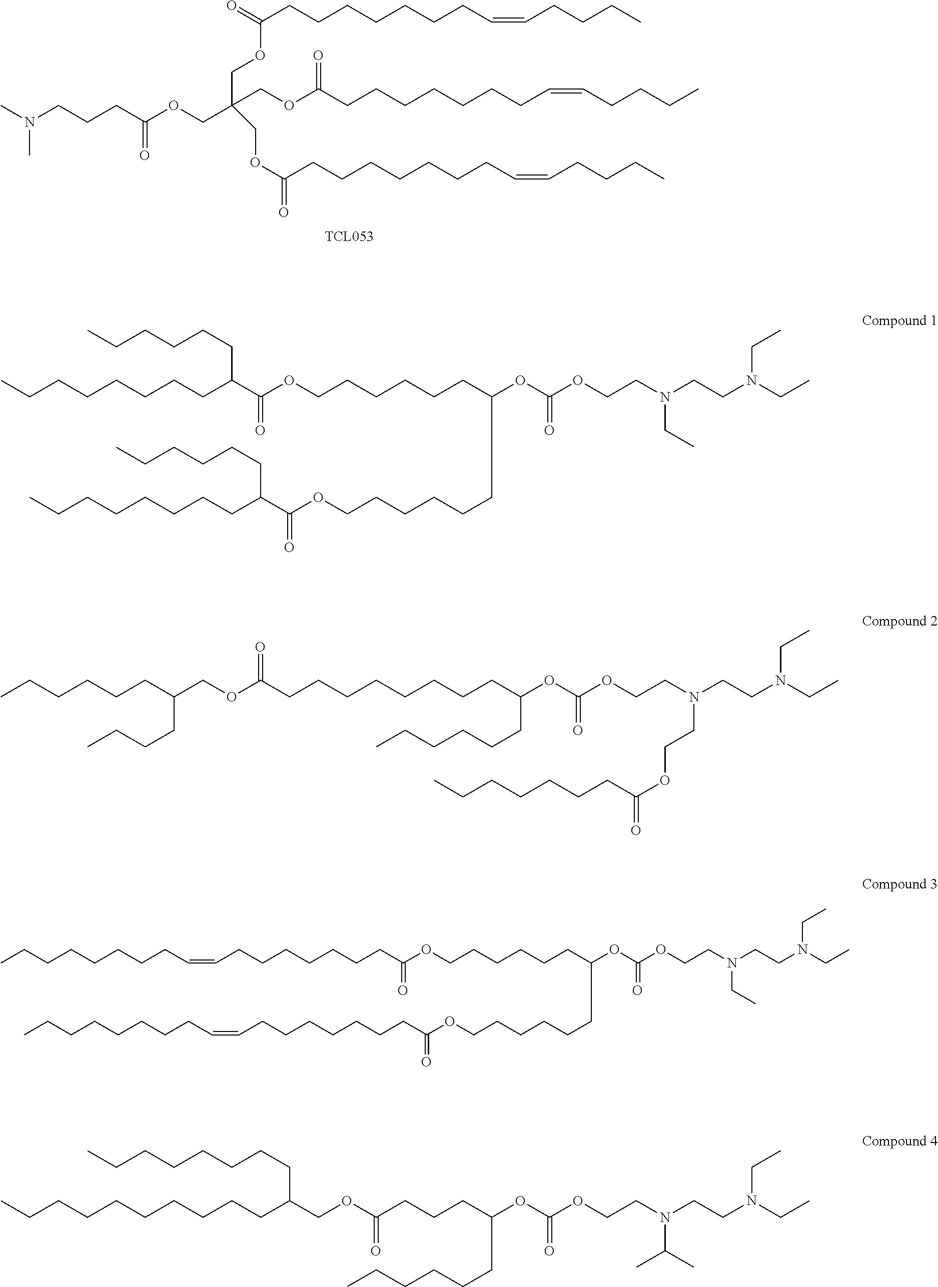

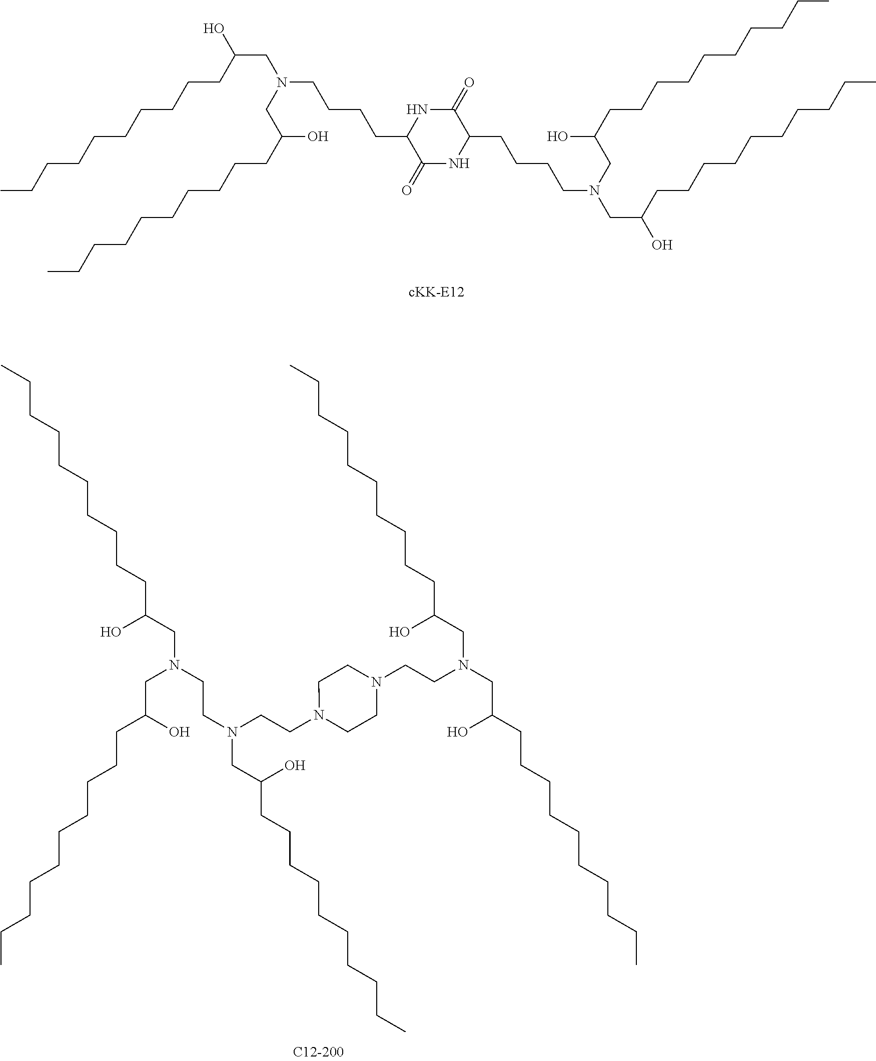

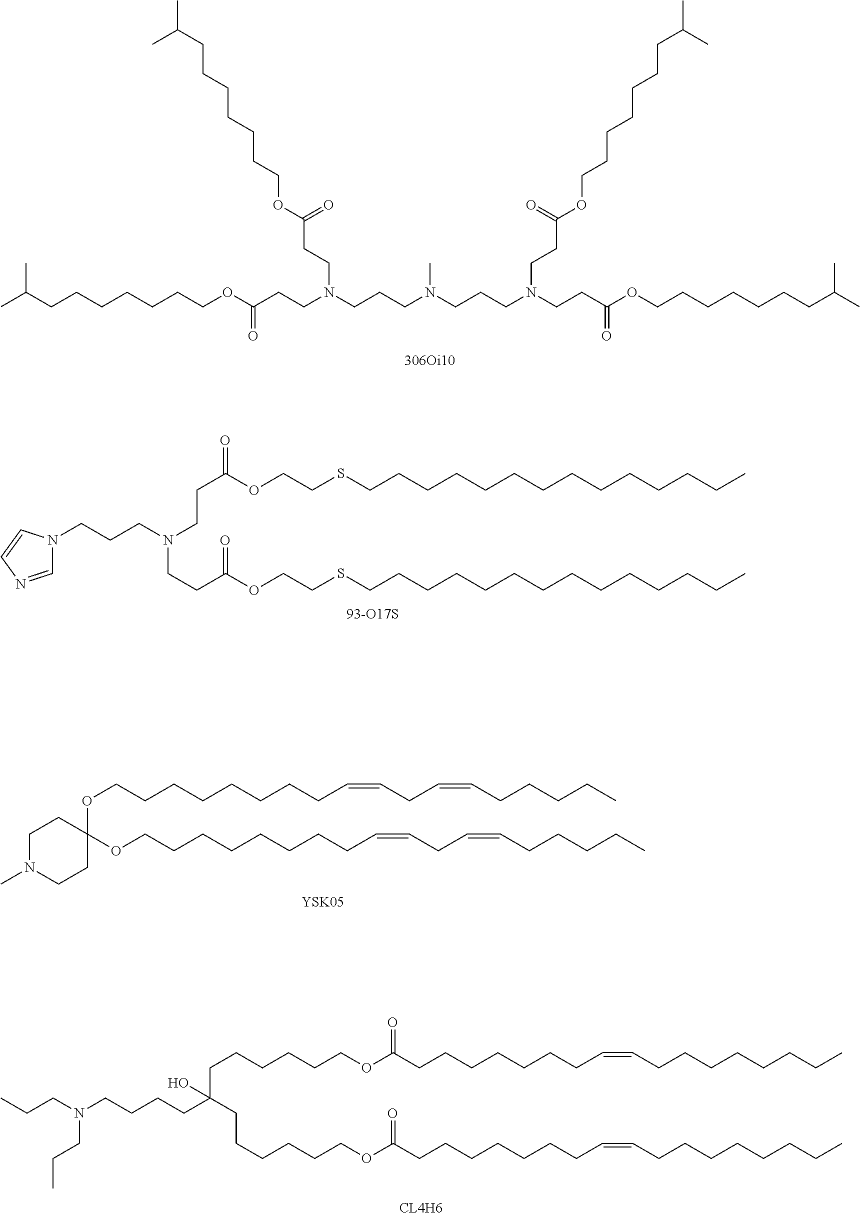

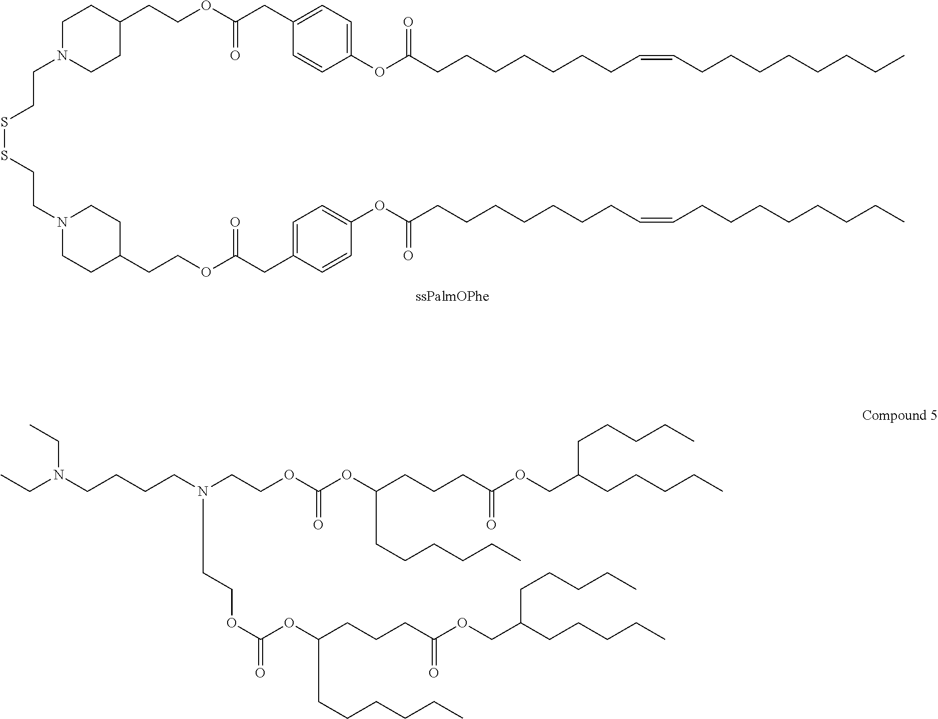

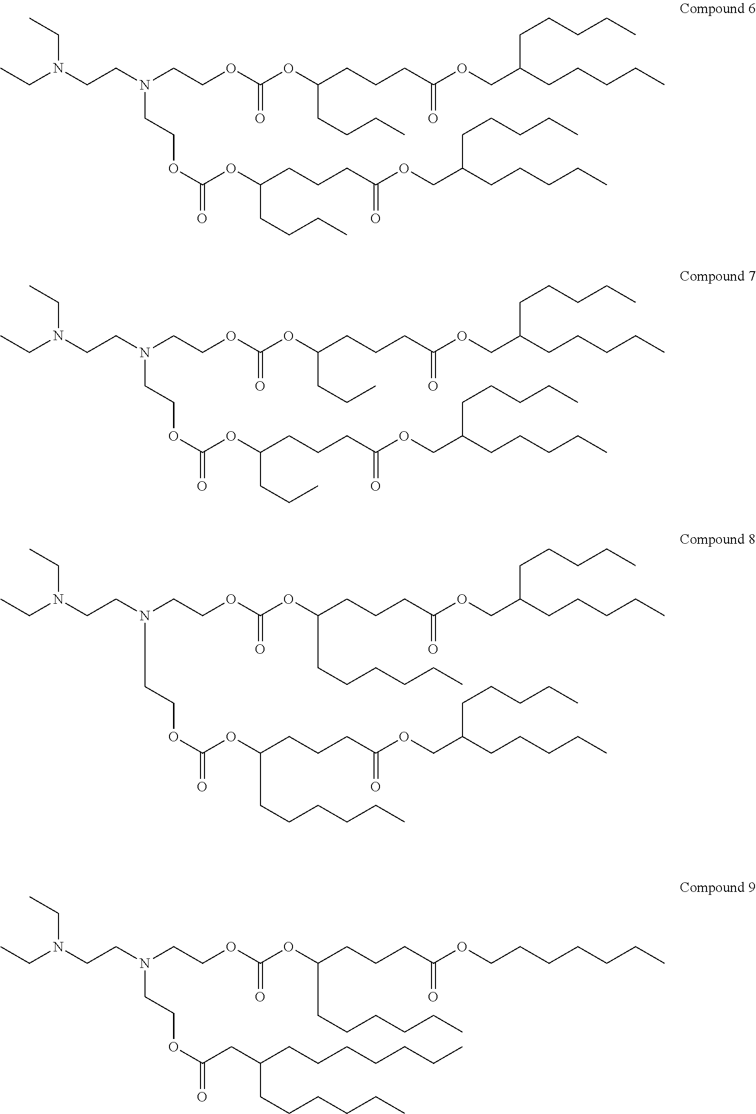

<8> The lipid composition of <1>, wherein the lipid nanoparticle comprises a sterol.
<9> The lipid composition of <1>, wherein the lipid nanoparticle comprises a phospholipid.
<10> The lipid composition of <1>, wherein the therapeutic agent comprises a polynucleotide.
<11> The lipid composition of <9>, wherein the polynucleotide is DNA or RNA.
<12> The lipid composition of <9>, wherein the polynucleotide is mRNA, sgRNA or siRNA.
   <13> The lipid composition of <1>, wherein the targeting molecule is at least one which is selected from nucleic acid, peptide, antibody and small molecule.
   <14> The lipid composition of <12>, wherein the targeting molecule is antibody.
   <15> The lipid composition of <1>, wherein the marker of hematopoietic stem/progenitor cells or mesenchymal stem cells is CD34, CD105, CD117, or CD184 (CXCR4).
   <16> The lipid composition of <1>, wherein the marker of hematopoietic stem/progenitor cells or mesenchymal stem cells is CD117.
   <17> The lipid composition of <1>, wherein the marker of mesenchymal stem cell is CD105.
   <18> A method for delivering a therapeutic agent to a cell which expresses a marker of hematopoietic stem/progenitor cells or mesenchymal stem cells, which comprises administering the lipid composition of <1> to a subject.
   <19> The method of <18>, which further comprises administering a therapeutically effective amount of an inflammatory reducing agent to the subject prior to administering the lipid composition of claim 1 to the subject.
   <20> The method of <19>, wherein the inflammatory reducing agents is selected from (a) corticosteroids, (b) antihistamines, (c) acetaminophen, (d) NSAIDS, (e) kinase inhibitors with CD117 kinase activity inhibiting activity, or (f) other immunosuppressants.
   <21> A method of reducing adverse effects related to anti-CD117 antibody-modified LNP administration, the method comprising administering a therapeutically effective amount of an inflammatory reducing agent to a subject prior to administering CD117 antibody-modified LNP.
   <22> The method of <21>, wherein the inflammatory reducing agents is selected from (a) corticosteroids, (b) antihistamines, (c) acetaminophen, (d) NSAIDS, (e) kinase inhibitors with CD117 kinase activity inhibiting activity, or (f) other immunosuppressants.

Methods of delivering therapeutic agents to hematopoietic stem/progenitor cell (HSPCs) or mesenchymal stem cells in culture are also provided herein.

### Effect of the Invention

According to the lipid composition and method of the present invention, a therapeutic agent can be efficiently delivered to the hematopoietic stem/progenitor cell or mesenchymal stem cells.

### BRIEF DESCRIPTION OF DRAWINGS

FIG. 1 shows in vitro evaluation of CD45 gene silencing on EML cells using LNPs conjugated with aCD117.

FIG. 2 shows knockdown of CD45 through aCD117-receptor interaction with ionizable lipids.

FIG. 3 shows CD45 expression level in bone marrow LSK cells as quantified by flow cytometry.

FIG. 4 shows in vivo Cre mRNA delivery in bone marrow HSPCs using Ai14 mouse model.

FIG. 5 shows LNP Uptake in HSPCs or LT-HSCs (Left) and level of functional gene silencing in HSPCs with different alkyl chain lengths (Right).

FIG. 6 shows uptake in HSPCs as determined by % of LSK cells that are DiR+ (Left) and functional knockdown of CD45 in HSPCs (Right).

FIG. 7 shows LNP uptake in various cell populations of the bone marrow as determined by % DiR positive (Top) and Dose response of functional siCD45 knockdown with Ab-LNP formulations (Bottom).

FIG. 8 shows (Top) LNP uptake in various cell populations of the bone marrow as determined by % DiR positive, and (Bottom) dose response of functional siCD45 knockdown with Ab-LNP formulations.

FIG. 9 shows (Left) Screening of alternate markers for in vivo HSPC delivery at a dose of 1 mg kg⁻¹ siCD45 (n=3 mice for anti-CD49d condition, n=2 mice for all other conditions, one-way ANOVA with Dunnett's multiple comparison test). Data are represented as mean±SD (*P<0.05, **P<0.01, ***P<0.001), and (Righ) Treatment of ligand decorated LNPs at a dose of 1 mg kg⁻¹ siCD45 does not result in any depletion or clearance of HSCs. Statistics determined by one-way ANOVA with Dunnett's multiple comparison to the PBS control group (n=3 mice for anti-CD49 group, n=2 mice for other groups). Significance was defined as P<0.05

FIG. 10 shows in vitro RNA delivery to human primary HSPC using a non-antagonistic antibody (Clone LMJ729).

FIG. 11 shows luciferase expression in human primary bone marrow CD34+ cells, which was quantified using SteadyGlo Luciferase Assay System after treatment with the LNPs or PBS for 24 h at a dose of 100 ng/5,000 cells.

FIG. 12 shows Itgb1 mRNA level in primary murine bone marrow mesenchymal stem cells which was quantified using RT-qPCR 24 hours post LNP treatment at 100 nM siRNA. Remaining Itgb1 mRNA level was normalized to housekeeping gene, B2m.

FIG. 13 shows luciferase expression in human primary bone marrow CD34+ cells which was quantified using using SteadyGlo Luciferase Assay System after treatment with the LNPs or PBS for 24 h at a dose of 100 ng/5,000 cells.

FIG. 14 shows that anti-CD117 LNPs encapsulating Cre mRNA shows high levels of editing in vivo. (a) Schematic of the Ai14 transgenic mouse LoxPflanked stop cassette preventing the transcription of TdTomato. Upon delivery of Cre recombinase via Cre mRNA, the stop cassette is excised, and the cell expresses TdTomato. (b) Timeline of experimental workflow for bone marrow and blood analysis. Panels a and b were created with BioRender.com. (c) Representative flow cytometry scatter plots of TdTomato expression in LT-HSCs with varying doses of Cre mRNA. (d) Quantification of the dose-response in both HSPCs and LT-HSCs (n=4 mice for 1 mg kg⁻¹ Ab-LNP group, n=3 mice for others). Statistics were performed with two-way ANOVA with Tukey's multiple comparison test. (e) Time course monitoring of TdTomato expression in mature immune cells (n=3 mice). (f) TdTomato expression in T cell subsets (n=3 mice). (g) TdTomato expression in erythrocytes (TER-119+) (n=3 mice). Data are represented as mean±SD (*P<0.05, **P<0.01, ***P<0.001).

FIG. 15 shows effect on PEG-lipid alkyl length on mRNA delivery to HSPCs in the bone marrow.

### DETAILED DESCRIPTION OF THE INVENTION

Hereinafter, the present invention will be described in detail.

In this specification, “˜” denotes a range including a numerical value described before and after it as a minimum value and a maximum value, respectively.

The present invention is a lipid composition comprising (A) a therapeutic agent and (B) a lipid nanoparticle bound to a targeting molecule,
wherein the therapeutic agent is encapsulated in lipid nanoparticles,
the lipid nanoparticle comprises an ionizable lipid, and
the targeting molecule is a molecule that specifically binds to a marker of hematopoietic stem/progenitor cells or mesenchymal stem cells.

In one embodiment, the lipid composition of the present invention is used in combination with an inflammatory reducing agent. The inflammatory reducing agents is selected from (a) corticosteroids, (b) antihistamines, (c) acetaminophen, (d) NSAIDS, (e) kinase inhibitors with CD117 kinase activity inhibiting activity, or (f) other immunosuppressants.

The present invention further relates to a method of delivering a therapeutic agent to a cell expressing a marker of hematopoietic stem/progenitor cells or mesenchymal stem cells, which comprises administering the lipid composition of the invention to a subject. Preferably, the method of delivering a therapeutic agent to a cell according to the present invention may further comprises administering a therapeutically effective amount of an inflammatory reducing agent to the subject prior to administering the lipid composition of the invention to the subject.

In the present invention, a lipid nanoparticle bound to a targeting molecule is used.

The present invention further relates to a method of reducing adverse effects related to anti-CD117 antibody-modified LNP administration, the method comprising administering a therapeutically effective amount of an inflammatory reducing agent to a subject prior to administering CD117 antibody-modified LNP. Preferably, the inflammatory reducing agents is selected from (a) corticosteroids, (b) antihistamines, (c) acetaminophen, (d) NSAIDS, (e) kinase inhibitors with CD117 kinase activity inhibiting activity, or (f) other immunosuppressants.

### Targeting Molecule

The targeting molecule is a molecule that specifically binds to a hematopoietic stem/progenitor cell marker or mesenchymal stem cells. By using a molecule that specifically bind to markers of hematopoietic stem/progenitor cells or mesenchymal stem cells as a targeting molecule, it becomes possible to efficiently deliver a therapeutic agent encapsulated in lipid nanoparticles to hematopoietic stem/progenitor cells or mesenchymal stem cells.

The type of targeting molecule is not particularly limited as long as it is a molecule that specifically binds to a marker of a hematopoietic stem/progenitor cell or mesenchymal stem cells, and may include a molecule that binds to a cell surface, a molecule that binds to an extracellular matrix, or the like. Molecules that bind to the cell surface can include, for example, molecules that bind to membrane proteins, such as receptors or channels that are exposed to the cell surface. As the targeting molecule, it is preferable to use a molecule which binds to a marker of hematopoietic stem/progenitor cells or mesenchymal stem cells. As the targeting molecule, it is preferable to use a non-antagonistic molecule. As the targeting molecule, for example, at least one selected from carbohydrates, nucleic acids, peptides, proteins, antibodies, antibody fragments, antigen binding domains, immunoglobulins or immunoglobulin fragments, and small molecules can be used. The targeting molecule is preferably an antibody.

The term “antibody,” as used herein, refers to an immunoglobulin molecule, which specifically binds with an antigen or epitope. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)2, as well as single chain antibodies, and humanized antibodies. The term “antibody fragment” refers to a portion of an intact antibody and refers to the antigenic determining variable regions of an intact antibody. Examples of antibody fragments include, but are not limited to, Fab, Fab′, F(ab′)2, VHH, and Fv fragments, linear antibodies, scFv antibodies, and multispecific antibodies formed from antibody fragments.

Markers for hematopoietic stem/progenitor cells can include CD13, CD27,,CD33, CD34, CD45, CD49d (VLA-4, integrin α4), CD49e (VLA-5, integrin α5), CD49f (VLA-6, integrin α6), CD51 (integrin αV), CD59, CD84 (CD150 family), CD93, CD110 (Thrombopoietin (TPO) receptor), CD114 (CSF3 receptor, G-CSF receptor), CD115 (CSF1 receptor), CD116 (GM-CSF receptor), CD117 (c-Kit/SCF receptor), CD121a (IL-1R), CD 123 (IL-3R), CD 124 (IL-4R), CD 125 (IL-5Rα), CD 126 (IL-6R), CD128 (IL-8Rα), CD129 (IL-9R), CD 133 (Prominin 1), CD135 (Flt3 receptor), CD166 (ALCAM), CD184(CXCR4), CD210 (IL-10R), CD218 (IL-18R1), CD244, CD360 (IL-21R), integrin β7, Prominin 2, Erythropoietin R, Endothelial Cell-Selective Adhesion Molecule, Tie1, Tie2, MPL, gp130, Leukemia inhibitory factor Receptor, oncostatin M receptor, Embigin, and the like. Among the above, CD117 is preferred. As an exemplary targeting molecule, anti-CD117 antibody can be used.

Hematopoietic stem/progenitor cells express c-Kit (CD117, a dimeric transmembrane receptor tyrosine kinase). Signaling involving CD117 is essential for the function of numerous hematopoietic stem/progenitor cells, including homing, proliferation, adhesion, maintain, and survival. CD117 is also expressed in other cell types, such as cancerous cells.

Markers for mesenchymal stem cells can include CD105.

Examples of antibody clones for each marker are shown below.

### Mouse CD117

Antagonistic: ACK2 (BioXCell)
Non-antagonistic: 2B8 (BioXCell)

### Human CD117

Antagonistic:
Briquilimab/AMG191/JSP191 (Amgen or Jasper therapeutics, WO2007/127317)
Barzolvolimab (Celldex, WO2022159737)
LMS359, GZQ167, LMJ451 (Clin Cancer Res (2018) 24 (17): 4297-4308. Novatris)
AB85/MGTA117 (Magenta therapeutics, WO2019084067, WO2020219748, WO2020219770, and WO2020219775)
Non-antagonistic
104D2 (Biolegend)
DLY884, LPG166, LQS721, LMJ729, LPG167, (Clin Cancer Res (2018) 24 (17): 4297-4308. Novartis)

### Human CD184(CXCR4)

Antagonistic:
Ulocuplumab (Bristol Meyers Squibb)
LY2510924 (Eli Lilly: peptide)
LY2624587 (Eli Lilly)
ALX-0651 (Sanofi/Ablynx: nanobody)
PF-06747143 (Pfizer)

### Human CD105

TRC105 (Tracon)

### Human CD34

8G12 (BD Biosciences)
581 (Biolegend)
S20016E (Biolegend)

### Human CD27

Agonistic (non-antagonistic):
Boserolimab (Merck/Aduro)
Varlilumab (Celldex)

### Human CD133

C-Mab43 (Monoclonal Antibodies in Immunodiagnosis and Immunotherapy. October 2017.231-235.)
HW350341.1 (GenBank: HW350341.1)

Although there is no particular limitation on the method of binding the targeting molecule to the lipid nanoparticles, it is preferable to bind the targeting molecule to any lipid component constituting the lipid nanoparticle. For example, when the lipid nanoparticles comprise an ionizable lipid, a sterol, a neutral lipid (e.g., a phospholipid, etc.), a lipid having a nonionic hydrophilic polymer (e.g., a lipid to which polyethylene glycol is bound, etc.), it is preferred to bind to any of the lipid components described above. As an example, a targeting molecule can be bound to a lipid having a nonionic hydrophilic polymer (e.g., a lipid to which polyethylene glycol is bound, etc.), but is not particularly limited.

The number of targeting molecule per lipid nanoparticle is not particularly limited, but in general one or more targeting molecule per lipid nanoparticle is preferred. In one embodiment, the number of targeting molecules per lipid nanoparticle can be determined by measuring the concentration of lipid nanoparticles, [A], by common methods (e.g., microfluidic resistive pulse sensing (MRPS) method, unable resistive pulse sensing (TRPS) method Nanoparticle Tracking Analysis (NTA), Transmission Electron Microscopy (TEM), etc. The concentration of the targeting molecule, [B], can be measured by common methods such as HPLC, BCA assay, Lowry assay, Bradford assay, etc.). The number of targeting molecules per lipid nanoparticle can be calculated as [B]/[A]. The concentration of lipid nanoparticles [A] can also be calculated from the volume-averaged particle size of lipid nanoparticles, the molecular volume of each constituent, and the molar ratio of each constituent.

### Sterols

The lipid nanoparticle preferably contains sterols.

In the present invention, since a sterol is contained, the membrane fluidity can be reduced and the effect to stabilize the lipid particles can be obtained.

The sterols are not particularly limited, and examples thereof include cholesterol, phytosterol (sitosterol, stigmasterol, fucosterol, spinasterol, brassicasterol, and the like), ergosterol, cholestanone, cholestenone, coprostanol, cholesteryl-2′-hydroxyethyl ether, cholesteryl-4′-hydroxybutyl ether, and the like. Among these, cholesterol is preferable.

The content of the sterols with respect to the total lipids is preferably 5 mol % to 80 mol %, more preferably 10 mol % to 80 mol %, still more preferably 10 mol % to 60 mol %, and further preferably 30 mol % to 50 mol %.

### Ionizable Lipids

In the present invention, an ionizable lipid is used. The ionizable lipid may be a lipid having at least one biodegradable group. The ionizable lipid may be a lipid having at least one ionizable amino group and at least one biodegradable group. The ionizable lipids are pH-responsive cationic lipids. They are electrically neutral at physiological pH such as in blood, and change to cationic in acidic environments such as endosomes. Examples of the above-mentioned biodegradable group include groups represented by —O(CO)O—, —O(CO)—, or —(CO)O—.

### Lipid Represented by Formula (4) or Salt Thereof

For example, a lipid represented by Formula (4) or a salt thereof may be used as the ionizable lipid.

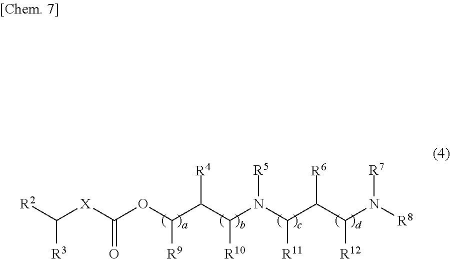

In the formula, X represents —NR¹— or —O—,
R¹ represents a hydrogen atom, a hydrocarbon group having 6 to 24 carbon atoms, or a group represented by R²¹-L¹-R²²—, where R²¹ represents a hydrocarbon group having 1 to 24 carbon atoms, L¹ represents —O(CO)O—, —O(CO)—, —(CO)O—, —O—, or a group represented by the following formula,

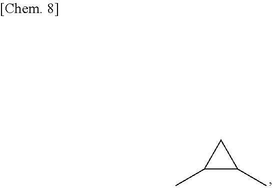

and R²² represents a divalent hydrocarbon linking group having 1 to 18 carbon atoms,
R² and R³ each independently represent a hydrogen atom, a hydrocarbon group having 3 to 24 carbon atoms, or a group represented by R³¹-L²-R³²—, where R³¹ represents a hydrocarbon group having 1 to 24 carbon atoms, L² represents —O(CO)O—, —O(CO)—, —(CO)O—, —O—, or a group represented by the following formula,

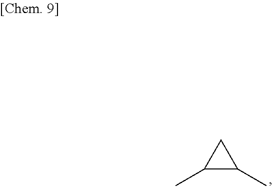

and R³² represents a divalent hydrocarbon linking group having 1 to 18 carbon atoms,
R⁴, R⁵, R⁶, R⁷, R⁸, R⁹, R¹⁰, R¹¹, and R¹² each independently represent a hydrogen atom or an alkyl group having 1 to 18 carbon atoms which may be substituted,
groups in any one or more pairs among R⁴ and R⁵, R¹⁰ and R⁵, R⁵ and R¹², R⁴ and R⁶, R⁵ and R⁶, R⁶ and R⁷, R⁶ and R¹⁰, R¹² and R⁷, and R⁷ and R⁸ may be linked to each other to form a 4- to 7-membered ring which may contain an O atom,
a substituent on the alkyl group having 1 to 18 carbon atoms which may be substituted is a hydroxyl group, a carboxyl group, an amino group represented by —NR⁴⁵R⁴⁶, a substituted or unsubstituted aryl group, a substituted or unsubstituted heteroaryl group, or a group represented by —O(CO)O—R⁴¹, —O(CO)—R⁴², —(CO)O—R⁴³, or —O—R⁴⁴, where R⁴¹, R⁴², R⁴³, R⁴⁴, R⁴⁵, and R⁴⁶ each independently represent a hydrocarbon group having 1 to 18 carbon atoms,
the substituent on the substituted or unsubstituted aryl group and on the substituted or unsubstituted heteroaryl group is an alkyl group having 1 to 18 carbon atoms, a hydroxyl group, a carboxyl group, an amino group represented by —NR⁴⁵R⁴⁶, or a group represented by —O(CO)O—R⁴¹, —O(CO)—R⁴², —(CO)O—R⁴³, or —O—R⁴⁴, where R⁴¹, R⁴², R⁴³, R⁴⁴, R⁴⁵, and R⁴⁶ each independently represent a hydrocarbon group having 1 to 18 carbon atoms, and
a, b, c, and d each independently represent an integer of 0 to 3, a+b is 1 or more, and c+d is 1 or more.

As the hydrocarbon group having 6 to 24 carbon atoms that is represented by R¹ and the hydrocarbon group having 3 to 24 carbon atoms that is represented by R² and R³, an alkyl group, an alkenyl group, or an alkynyl group is preferable, and an alkyl group or an alkenyl group is more preferable. The alkyl group having 6 to 24 carbon atoms and the alkyl group having 3 to 24 carbon atoms may be linear or branched or may be chainlike or cyclic. The alkyl group having 6 to 24 carbon atoms is preferably an alkyl group having 6 to 20 carbon atoms, and the alkyl group having 3 to 24 carbon atoms is more preferably an alkyl group having 6 to 20 carbon atoms. Specifically, examples thereof include a hexyl group, a heptyl group, an octyl group, a nonyl group, a decyl group, an undecyl group, a dodecyl group, a tridecyl group, a trimethyldodecyl group (preferably a 3,7,11-trimethyldodecyl group), a tetradecyl group, a pentadecyl group, a hexadecyl group, a tetramethylhexadecyl group (preferably a 3,7,11,15-tetramethylhexadecyl group), a heptadecyl group, an octadecyl group, a nonadecyl group, an icosyl group, and the like. The alkenyl group having 6 to 24 carbon atoms and the alkenyl group having 3 to 24 carbon atoms may be linear or branched or may be chainlike or cyclic. The alkenyl group having 6 to 24 carbon atoms is preferably an alkenyl group having 6 to 20 carbon atoms, and the alkenyl group having 3 to 24 carbon atoms is more preferably an alkenyl group having 6 to 20 carbon atoms. Specifically, examples thereof include a hexenyl group, a heptenyl group, an octenyl group, a nonenyl group, a decenyl group, an undecenyl group, a dodecenyl group, a dodecadienyl group, a tridecenyl group, a tetradecenyl group, a pentadecenyl group, a hexadecenyl group (preferably a (Z)-hexadec-9-enyl group), a hexadecadienyl group, a heptadecenyl group (preferably a (Z)-heptadec-8-enyl group), a heptadecadienyl group (preferably a (8Z,11Z)-heptadeca-8,11-dienyl group), an octadecenyl group (preferably a (Z)-octadec-9-enyl group), an octadecadienyl group (preferably a (9Z,12Z)-octadeca-9,12-dienyl group), a nonadecenyl group, an icosenyl group (preferably a (Z)-icos-11-enyl group), an icosadienyl group (preferably a (11Z,14Z)-icosa-11,14-dienyl group), and the like. The alkynyl group having 6 to 24 carbon atoms is preferably an alkynyl group having 6 to 20 carbon atoms, and the alkynyl group having 3 to 24 carbon atoms is more preferably an alkynyl group having 6 to 20 carbon atoms. Specifically, examples thereof include a hexynyl group, a heptynyl group, an octynyl group, a nonynyl group, a decynyl group, an undecynyl group, a dodecynyl group, a tetradecynyl group, a pentadecynyl group, a hexadecynyl group, a heptadecynyl group, an octadecynyl group, and the like. All of the above alkenyl groups preferably have one double bond or two double bonds. All of the above alkynyl groups preferably have one triple bond or two triple bonds.

The hydrocarbon group having 1 to 24 carbon atoms that is represented by R²¹ and R³¹ is preferably an alkyl group having 10 to 24 carbon atoms, an alkenyl group having 10 to 24 carbon atoms, or an alkynyl group having 10 to 24 carbon atoms. The alkyl group having 10 to 24 carbon atoms may be linear or branched or may be chainlike or cyclic. The alkyl group having 10 to 24 carbon atoms is preferably an alkyl group having 12 to 24 carbon atoms. Specifically, examples thereof include a decyl group, an undecyl group, a dodecyl group, a tridecyl group, a trimethyldodecyl group (preferably a 3,7,11-trimethyldodecyl group), a tetradecyl group, a pentadecyl group, a hexadecyl group, a tetramethylhexadecyl group (preferably a 3,7,11,15-tetramethylhexadecyl group), a heptadecyl group, an octadecyl group, a 2-butylhexyl group, a 2-butyloctyl group, a 1-pentylhexyl group, a 2-pentylheptyl group, a 3-pentyloctyl group, a 1-hexylheptyl group, a 1-hexylnonyl group, a 2-hexyloctyl group, a 2-hexyldecyl group, a 3-hexylnonyl group, a 1-heptyloctyl group, a 2-heptylnonyl group, a 2-heptylundecyl group, a 3-heptyldecyl group, a 1-octylnonyl group, a 2-octyldecyl group, a 2-octyldodecyl group, a 3-octylundecyl group, a 2-nonylundecyl group, a 3-nonyldodecyl group, a 2-decyldodecyl group, a 2-decyltetradecyl group, a 3-decyltridecyl group, a 2-(4,4-dimethylpentan-2-yl)-5,7,7-trimethyloctyl group, and the like. The alkenyl group having 10 to 24 carbon atoms may be linear or branched or may be chainlike or cyclic. Specifically, examples thereof include a decenyl group, an undecenyl group, a dodecenyl group, a dodecadienyl group, tridecenyl group (preferably a (Z)-tridec-8-enyl group), a tetradecenyl group (preferably a tetradec-9-enyl group), a pentadecenyl group (preferably a (Z)-pentadec-8-enyl group), a hexadecenyl group (preferably a (Z)-hexadec-9-enyl group), a hexadecadienyl group, a heptadecenyl group (preferably a (Z)-heptadec-8-enyl group), a heptadecadienyl group (preferably a (8Z,11Z)-heptadeca-8,11-dienyl group), an octadecenyl group (preferably a (Z)-octadec-9-enyl group), an octadecadienyl group (preferably a (9Z,12Z)-octadeca-9,12-dienyl group), and the like. The alkynyl group having 10 to 24 carbon atoms may be linear or branched or may be chainlike or cyclic. Specifically, examples thereof include a decynyl group, an undecynyl group, a dodecynyl group, a tetradecynyl group, a pentadecynyl group, a hexadecynyl group, a heptadecynyl group, an octadecynyl group, and the like. All of the above alkenyl groups preferably have one double bond or two double bonds. All of the above alkynyl groups preferably have one triple bond or two triple bonds.

The divalent hydrocarbon linking group having 1 to 18 carbon atoms that is represented by R²² and R³² is preferably an alkylene group having 1 to 18 carbon atoms or an alkenylene group having 2 to 18 carbon atoms. The alkylene group having 1 to 18 carbon atoms may be linear or branched or may be chainlike or cyclic. The number of carbon atoms in the alkylene group is preferably 1 to 12, more preferably 1 to 10, and still more preferably 2 to 10. Specifically, examples thereof include a methylene group, an ethylene group, a trimethylene group, a tetramethylene group, a pentamethylene group, a hexamethylene group, a heptamethylene group, an octamethylene group, a nonamethylene group, a decamethylene group, an undecamethylene group, a dodecamethylene group, and the like. The alkenylene group having 2 to 18 carbon atoms may be linear or branched or may be chainlike or cyclic. The number of carbon atoms in the alkenylene group is preferably 1 to 12, and more preferably 2 to 10.

—O(CO)O—, —O(CO)—, and —(CO)O— are in a preferred range of L¹, and —O(CO)— and —(CO)O— are in a more preferred range of L¹.

—O(CO)O—, —O(CO)—, and —(CO)O— are in a preferred range of L², and —O(CO)— and —(CO)O— are in a more preferred range of L².

The alkyl group having 1 to 18 carbon atoms which may be substituted and which represented by R⁴, R⁶, R⁹, R¹⁰, R¹¹, and R¹² may be linear or branched or may be chainlike or cyclic. The number of carbon atoms in the alkyl group is preferably 1 to 12. Specifically, examples thereof include a methyl group, an ethyl group, a propyl group, an isopropyl group, a cyclopropyl group, a butyl group, an isobutyl group, a tert-butyl group, a cyclobutyl group, a pentyl group, a cyclopentyl group, a hexyl group, a cyclohexyl group, a heptyl group, an octyl group, a nonyl group, a decyl group, an undecyl group, a dodecyl group, and the like. In a case where the alkyl group has a substituent, as the substituent, a hydroxyl group, a carboxyl group, or a group represented by —O(CO)O—R⁴¹, —O(CO)—R⁴², —(CO)O—R⁴³, or —O—R⁴⁴ is preferable, and a group represented by —O(CO)—R⁴² or —(CO)O—R⁴³ is more preferable.

The alkyl group having 1 to 18 carbon atoms which may be substituted and which represented by R⁵, R⁷, and R⁸ may be linear or branched or may be chainlike or cyclic. The number of carbon atoms in the alkyl group is preferably 1 to 12, and more preferably 1 to 8. Specifically, examples thereof include a methyl group, an ethyl group, a propyl group, an isopropyl group, a cyclopropyl group, a butyl group, an isobutyl group, a tert-butyl group, a cyclobutyl group, a pentyl group, a cyclopentyl group, a hexyl group, a cyclohexyl group, a heptyl group, an octyl group, a nonyl group, a decyl group, an undecyl group, a dodecyl group, and the like. In a case where the alkyl group has a substituent, as the substituent, a hydroxyl group, a carboxyl group, or a group represented by —O(CO)O—R⁴¹, —O(CO)—R⁴², —(CO)O—R⁴³, or —O—R⁴⁴ is preferable, and a group represented by —O(CO)—R⁴², —(CO)O—R⁴³, or —O—R⁴⁴ is more preferable.

Examples of the 4- to 7-membered ring which may contain an O atom include an azetidine ring, a pyrrolidine ring, a piperidine ring, a morpholine ring, and an azepane ring. The 4- to 7-membered ring is preferably a 6-membered ring and is preferably a piperidine ring or a morpholine ring.

In a case where the alkyl group having 1 to 18 carbon atoms which is represented by R⁴, R⁵, R⁶, R⁷, R⁸, R⁹, R¹⁰, R¹¹, and R¹² and which may be substituted has a substituted or unsubstituted aryl group as a substituent, the number of carbon atoms in the aryl group is preferably 6 to 22, more preferably 6 to 18, and still more preferably 6 to 10. Specifically, examples of the aryl group include a phenyl group, a naphthyl group, an anthracenyl group, a phenanthrenyl group, and the like. As the substituent on the aryl group, an alkyl group having 1 to 18 carbon atoms, a hydroxyl group, a carboxyl group, an amino group represented by —NR⁴⁵R⁴⁶, or a group represented by —O(CO)O—R⁴¹, —O(CO)—R⁴², —(CO)O—R⁴³, or —O—R⁴⁴ is preferable, and a hydroxyl group or a carboxyl group is more preferable. Specifically, examples of the substituted aryl group include a hydroxyphenyl group, a carboxyphenyl group, and the like.

In a case where the alkyl group having 1 to 18 carbon atoms which is represented by R⁴, R⁵, R⁶, R⁷, R⁸, R⁹, R¹⁰, R¹¹, and R¹² and which may be substituted has a substituted or unsubstituted heteroaryl group as a substituent, the number of carbon atoms in the heteroaryl group is preferably 1 to 12, and more preferably 1 to 6. Specifically, examples of the heteroaryl group include a pyridyl group, a pyrazolyl group, an imidazolyl group, a benzimidazolyl group, a thiazolyl group, an oxazolyl group, and the like. As the substituent on the heteroaryl group, an alkyl group having 1 to 18 carbon atoms, a hydroxyl group, a carboxyl group, an amino group represented by —NR⁴⁵R⁴⁶, or a group represented by —O(CO)O—R⁴¹, —O(CO)—R⁴², —(CO)O—R⁴³, or —O—R⁴⁴ is preferable, and a hydroxyl group or a carboxyl group is more preferable. Specifically, examples of the substituted or unsubstituted heteroaryl group include a hydroxypyridyl group, a carboxypyridyl group, a pyridonyl group, and the like.

As hydrocarbon group having 1 to 18 carbon atoms that is represented by R⁴¹, R⁴², R⁴³, R⁴⁴, R⁴⁵, and R⁴⁶, an alkyl group having 1 to 18 carbon atoms, an alkenyl group having 2 to 18 carbon atoms, or an alkynyl group having 2 to 18 carbon atoms is preferable, and an alkyl group having 1 to 18 carbon atoms or an alkenyl group having 2 to 18 carbon atoms is more preferable. The alkyl group having 1 to 18 carbon atoms may be linear or branched or may be chainlike or cyclic. The number of carbon atoms in the alkyl group is preferably 3 to 18, and more preferably 5 to 18. Specifically, examples thereof include a propyl group, an isopropyl group, a cyclopropyl group, a butyl group, an isobutyl group, a tert-butyl group, a cyclobutyl group, a pentyl group, a cyclopentyl group, a hexyl group, a cyclohexyl group, a heptyl group, an octyl group, a nonyl group, a decyl group, an undecyl group, a dodecyl group, a tridecyl group, a trimethyldodecyl group (preferably a 3,7,11-trimethyldodecyl group), a tetradecyl group, a pentadecyl group, a hexadecyl group, a heptadecyl group, an octadecyl group, and the like. The alkenyl group having 2 to 18 carbon atoms may be linear or branched or may be chainlike or cyclic. The number of carbon atoms in the alkenyl group is preferably 3 to 18, and more preferably 5 to 18. Specifically, examples thereof include an allyl group, a prenyl group, a pentenyl group, a hexenyl group, a heptenyl group, an octenyl group, a nonenyl group (preferably a (Z)-2-nonenyl group or an (E)-2-nonenyl group), a decenyl group, an undecenyl group, a dodecenyl group, a dodecadienyl group, a tridecenyl group (preferably a (Z)-tridec-8-enyl group), a tetradecenyl group (preferably a tetradec-9-enyl group), a pentadecenyl group (preferably a (Z)-pentadec-8-enyl group), a hexadecenyl group (preferably a (Z)-hexadec-9-enyl group), a hexadecadienyl group, a heptadecenyl group (preferably a (Z)-heptadec-8-enyl group), a heptadecadienyl group (preferably a (8Z,11Z)-heptadeca-8,11-dienyl group), an octadecenyl group (preferably a (Z)-octadec-9-enyl group), an octadecadienyl group (preferably a (9Z,12Z)-octadeca-9,12-dienyl group), and the like. The alkynyl group having 2 to 18 carbon atoms may be linear or branched or may be chainlike or cyclic. The number of carbon atoms in the alkynyl group is preferably 3 to 18, and more preferably 5 to 18. Specifically, examples thereof include a propargyl group, a butynyl group, a pentynyl group, a hexynyl group, a heptynyl group, an octynyl group, a nonynyl group, a decynyl group, an undecynyl group, a dodecynyl group, a tetradecynyl group, a pentadecynyl group, a hexadecynyl group, a heptadecynyl group, an octadecynyl group, and the like.

In a case where X represents —NR¹—, R¹ preferably represents a hydrocarbon group having 6 to 24 carbon atoms or a group represented by R²¹-L¹-R²²—. In this case, it is preferable that one of R² and R³ represent a hydrogen atom and the other represent a hydrocarbon group having 6 to 24 carbon atoms or a group represented by R³¹-L²-R³²—.

In a case where X represents —O—, it is preferable that R² and R³ each independently represent a hydrocarbon group having 6 to 24 carbon atoms or a group represented by R³¹-L²-R³²—.

It is preferable that R⁴, R⁶, R⁹, R¹⁰, R¹¹, and R¹² each represent a hydrogen atom.

R⁵ is preferably a hydrogen atom, an alkyl group having 1 to 18 carbon atoms, an alkyl group having 1 to 18 carbon atoms which may be substituted with —O(CO)—R⁴² or —(CO)O—R⁴³, an alkyl group having 1 to 18 carbon atoms which may be substituted with an aryl group, or an alkyl group having 1 to 18 carbon atoms which may be substituted with a hydroxyl group. In a case where R⁵ is an alkyl group, R⁵ may be linked to R⁴, R⁶, R¹⁰, and R¹² to form a ring which may contain an O atom. Particularly, R⁵ is preferably an alkyl group having 1 to 18 carbon atoms, an alkyl group having 1 to 18 carbon atoms which may be substituted with —O(CO)—R⁴² or —(CO)O—R⁴³, an alkyl group having 1 to 12 carbon atoms which may be substituted with an aryl group, or an alkyl group having 1 to 8 carbon atoms which may be substituted with a hydroxyl group, and more preferably an alkyl group having 1 to 18 carbon atoms or an alkyl group having 1 to 18 carbon atoms which may be substituted with —O(CO)—R⁴² or —(CO)O—R⁴³.

R⁷ and R⁸ preferably each independently represent a hydrogen atom, a hydrocarbon group having 1 to 18 carbon atoms, an alkyl group having 1 to 18 carbon atoms which may be substituted with —O(CO)—R⁴² or —(CO)O—R⁴³, an alkyl group having 1 to 8 carbon atoms which may be substituted with an aryl group, or an alkyl group having 1 to 8 carbon atoms which may be substituted with a hydroxyl group. Alternatively, it is preferable that R⁷ and R⁸ be linked to each other to form a 4- to 7-membered ring which may contain an O atom.

R⁵ is not linked to R⁷ or R⁸ and does not form a ring with R⁷ or R⁸.

a+b is preferably 1 or 2, and more preferably 1. c+d is preferably 1 or 2, and more preferably 1.

The compound represented by Formula (4) is preferably a compound represented by Formula (21).

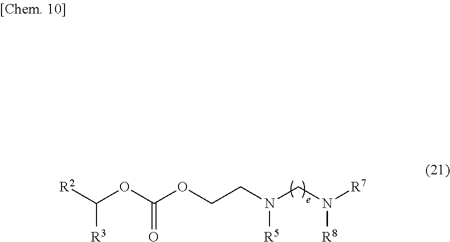

In the formula, R² and R³ each independently represent a hydrocarbon group containing one or more unsaturated bond and having 3 to 24 carbon atoms, or R² and R³ each independently represent a group represented by R³¹-L²-R³²—, or one of R² and R³ represents a group represented by R³¹-L²-R³²— and the other represents a hydrocarbon group having 3 to 24 carbon atoms,
R³¹ represents a hydrocarbon group having 1 to 24 carbon atoms,
L² represents —O(CO)O—, —O(CO)—, —(CO)O—, —O—, or a group represented by the following formula,

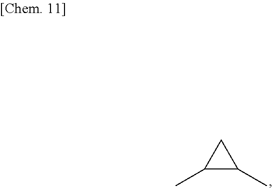

and R³² represents a divalent hydrocarbon linking group having 1 to 18 carbon atoms,
R⁵ represents an alkyl group having 1 to 18 carbon atoms which may be substituted with —O(CO)—R⁴² or —(CO)O—R⁴³ where R⁴² and R⁴³ each independently represent a hydrocarbon group having 1 to 18 carbon atoms,
R⁷ and R⁸ each independently represent an alkyl group having 1 to 4 carbon atoms
e represents 2 or 3.

In formula (21), preferably one of R² and R³ is a group represented by R³¹-L²-R³²—, and the other is a hydrocarbon group having 3 to 24 carbon atoms. In formula (21), L2 preferably represents —O(CO)— or —(CO)O—.

The compound represented by Formula (4) may form a salt.

Examples of the salt in a basic group include salts with mineral acids such as hydrochloric acid, hydrobromic acid, nitric acid, and sulfuric acid; salts with organic acid, succinic acid, malic acid, tartaric acid, aspartic acid, trichloroacetic acid, and trifluoroacetic acid; and salts with sulfonic acids such as methanesulfonic acid, benzenesulfonic acid, p-toluenesulfonic acid, mesitylenesulfonic acid, and naphthalenesulfonic acid.

Examples of the salt in an acidic group include salts with alkali metals such as sodium and potassium; salts with alkaline earth metals such as calcium and magnesium; ammonium salts; salts with nitrogen-containing organic bases such as trimethylamine, triethylamine, tributylamine, pyridine, N,N-dimethylaniline, N-methylpiperidine, N-methylmorpholine, diethylamine, dicyclohexylamine, procaine, dibenzylamine, N-benzyl-β-phenethylamine, 1-ephenamine, and N,N′-dibenzylethylenediamine, and the like.

Among the above salts, for example, pharmacologically acceptable salts are preferable.

The lipid represented by the formula (4) and a method for producing the same are described in WO2019/235635A and WO2021/095876A, the entire of which are incorporated herein by reference.

### Lipid Represented by Formula (1) or Salt Thereof

As another example, a lipid represented by Formula (1) or a salt thereof may be used as the ionizable lipid.

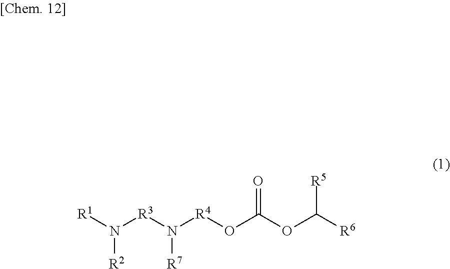

In the formula,
R¹ and R² each independently represent a hydrocarbon group having 1 to 18 carbon atoms, and R³ represents a hydrocarbon group having 2 to 8 carbon atoms, where the hydrocarbon groups represented by R¹, R², and R³ may be substituted with one or more substituents selected from —OH, COOH, —NR⁵¹R⁵², —OC(O)O—R⁵³, —C(O)O—R⁵⁴, —OC(O)—R⁵⁵, and —O—R⁵⁶,
R⁴ represents a hydrocarbon group having 1 to 8 carbon atoms,
R⁵ and R⁶ each independently represent a hydrocarbon group having 1 to 8 carbon atoms or —R⁸-L¹-R⁹, excluding a case that both R⁵ and R⁶ are hydrocarbon groups having 1 to 8 carbon atoms,
R⁷ represents —R¹⁰-L²-R¹¹-L³-R¹²,
R⁵¹ and R⁵² each independently represent a hydrocarbon group having 1 to 8 carbon atoms,
R⁵³, R⁵⁴, R⁵⁵, and R⁵⁶ each independently represent a hydrocarbon group having 1 to 24 carbon atoms,
the hydrocarbon groups represented by R⁵³, R⁵⁴, R⁵⁵, and R⁵⁶ may be substituted with an aryl group having 6 to 20 carbon atoms or —S—R⁵⁸,
the above-described aryl group having 6 to 20 carbon atoms may be substituted with —OH, COOH, —NR⁵¹R⁵², —OC(O)O—R⁵³, —C(O)O—R⁵⁴, —OC(O)—R⁵⁵, —O—R⁵⁶, or -(hydrocarbon group having 1 to 12 carbon atoms)-R⁵⁷,
R⁵⁸ represents a hydrocarbon group having 1 to 12 carbon atoms, and
R⁵⁷ represents —OH, COOH, —NR⁶¹R⁶², —OC(O)O—R⁶³, —C(O)O—R⁶⁴, —OC(O)—R⁶⁵, or —O—R⁶⁶.
R⁶¹ and R⁶² each independently represent a hydrocarbon group having 1 to 8 carbon atoms,
R⁶³, R⁶⁴, R⁶⁵, and R⁶⁶ each independently represent a hydrocarbon group having 1 to 24 carbon atoms,
the hydrocarbon groups represented by R⁶³, R⁶⁴, R⁶⁵, and R⁶⁶ may be substituted with an aryl group having 6 to 20 carbon atoms or —S—R⁶⁸,
the above-described aryl group having 6 to 20 carbon atoms may be substituted with —OH, COOH, —NR⁶¹R⁶², —OC(O)O—R⁶³, —C(O)O—R⁶⁴, —OC(O)—R⁶⁵, —O—R⁶⁶, or -(hydrocarbon group having 1 to 12 carbon atoms)-R⁶⁷,
R⁶⁸ represents a hydrocarbon group having 1 to 12 carbon atoms, and
L¹, L², and L³ each independently represent —OC(O)O—, —C(O)O—, —OC(O)—, or —O—.
R⁸ represents a hydrocarbon group having 1 to 12 carbon atoms,
R⁹ represents a hydrocarbon group having 1 to 24 carbon atoms,
R¹⁰ represents a hydrocarbon group having 1 to 8 carbon atoms,
R¹¹ represents a hydrocarbon group having 1 to 24 carbon atoms,
R¹² represents a hydrocarbon group having 1 to 24 carbon atoms,
the hydrocarbon groups represented by R⁹ and R¹² may be substituted with an aryl group, —OC(O)O—R⁵³, —C(O)O—R⁵⁴, —OC(O)—R⁵⁵, or —S—R⁵⁸, where definitions of R⁵³, R⁵⁴, R⁵⁵, and R⁵⁸ are as described above, and
the hydrocarbon group represented by R¹¹ may be substituted with —OC(O)O—R⁵³, —C(O)O—R⁵⁴, or —OC(O)—R⁵⁵, where the definitions of R⁵³, R⁵⁴, and R⁵⁵ are as described above.

A hydrocarbon group having 1 to 24 carbon atoms, a hydrocarbon group having 1 to 18 carbon atoms, a hydrocarbon group having 1 to 12 carbon atoms, a hydrocarbon group having 2 to 8 carbon atoms, and a hydrocarbon group having 1 to 8 carbon atoms are each preferably an alkyl group, an alkenyl group, or an alkynyl group.

The alkyl group may be linear or branched, or may be chainlike or cyclic. Specifically, examples of the alkyl group include a methyl group, an ethyl group, a propyl group, an isopropyl group, a cyclopropyl group, a butyl group, an isobutyl group, a tert-butyl group, a cyclobutyl group, a pentyl group, a cyclopentyl group, a hexyl group, a cyclohexyl group, a heptyl group, an octyl group, a nonyl group, a decyl group, an undecyl group, a dodecyl group, a tridecyl group, a trimethyldodecyl group (preferably a 3,7,11-trimethyldodecyl group), a tetradecyl group, a pentadecyl group, a hexadecyl group, a tetramethylhexadecyl group (preferably a 3,7,11,15-tetramethylhexadecyl group), a heptadecyl group, an octadecyl group, a 2-butylhexyl group, a 2-butyloctyl group, a 1-pentylhexyl group, a 2-pentylheptyl group, a 3-pentyloctyl group, a 1-hexylheptyl group, a 1-hexylnonyl group, a 2-hexyloctyl group, a 2-hexyldecyl group, a 3-hexylnonyl group, a 1-heptyloctyl group, a 2-heptylnonyl group, a 2-heptylundecyl group, a 3-heptyldecyl group, a 1-octylnonyl group, a 2-octyldecyl group, a 2-octyldodecyl group, a 3-octylundecyl group, a 2-nonylundecyl group, a 3-nonyldodecyl group, a 2-decyldodecyl group, a 2-decyltetradecyl group, a 3-decyltridecyl group, a 2-(4,4-dimethylpentan-2-yl)-5,7,7-trimethyloctyl group, and the like.

The alkenyl group may be linear or branched, or may be chainlike or cyclic. Specifically, examples of the alkenyl group include an allyl group, a prenyl group, a pentenyl group, a hexenyl group, a heptenyl group, an octenyl group, a nonenyl group (preferably a (Z)-2-nonenyl group or an (E)-2-nonenyl group), a decenyl group, an undecenyl group, a dodecenyl group, a dodecadienyl group, a tridecenyl group (preferably a (Z)-tridec-8-enyl group), a tetradecenyl group (preferably a tetradec-9-enyl group), a pentadecenyl group (preferably a (Z)-pentadec-8-enyl group), a hexadecenyl group (preferably a (Z)-hexadec-9-enyl group), a hexadecadienyl group, a heptadecenyl group (preferably a (Z)-heptadec-8-enyl group), a heptadecadienyl group (preferably a (8Z,11Z)-heptadeca-8,11-dienyl group), an octadecenyl group (preferably a (Z)-octadec-9-enyl group), an octadecadienyl group (preferably a (9Z,12Z)-octadeca-9,12-dienyl group), and the like.

The alkynyl group may be linear or branched, or may be chainlike or cyclic. Specifically, examples of alkynyl group include a propargyl group, a butynyl group, a pentynyl group, a hexynyl group, a heptynyl group, an octynyl group, a nonynyl group, a decynyl group, an undecynyl group, a dodecynyl group, a tetradecynyl group, a pentadecynyl group, a hexadecynyl group, a heptadecynyl group, an octadecynyl group, and the like.

All of the above alkenyl groups preferably have one double bond or two double bonds. All of the above alkynyl groups preferably have one triple bond or two triple bonds.

The hydrocarbon group having 1 to 12 carbon atoms in -(hydrocarbon group having 1 to 12 carbon atoms)-R⁶⁷ is preferably an alkylene group having 1 to 12 carbon atoms or an alkenylene group having 2 to 12 carbon atoms. The alkylene group having 1 to 12 carbon atoms and the alkenylene group having 2 to 12 carbon atoms may be linear or branched, or may be chainlike or cyclic.

Specifically, examples thereof include a methylene group, an ethylene group, a trimethylene group, a tetramethylene group, a pentamethylene group, a hexamethylene group, a heptamethylene group, an octamethylene group, a nonamethylene group, a decamethylene group, an undecamethylene group, and the like.

The aryl group preferably has 6 to 20 carbon atoms, more preferably has 6 to 18 carbon atoms, and even more preferably 6 to 10 carbon atoms. Specifically, examples of the aryl group include a phenyl group, a naphthyl group, an anthracenyl group, a phenanthrenyl group, and the like.

R¹ and R² each independently preferably represent a hydrocarbon group having 1 to 12 carbon atoms, more preferably represent a hydrocarbon group having 1 to 6 carbon atoms, and even more preferably represent a hydrocarbon group having 1 to 3 carbon atoms.

R³ preferably represents a hydrocarbon group having 2 to 6 carbon atoms and more preferably represents a hydrocarbon group having 2 to 4 carbon atoms.

The hydrocarbon groups represented by R¹, R², and R³ may be preferably substituted with —OH.

L¹ and L³ each independently preferably represent —C(O)O— or —OC(O)—.

L² preferably represents —OC(O)O—, —C(O)O—, or —OC(O)—.

R⁸ preferably represents a hydrocarbon group having 1 to 10 carbon atoms and more preferably represents a hydrocarbon group having 1 to 8 carbon atoms.

R⁹ preferably represents a hydrocarbon group having 1 to 20 carbon atoms and more preferably represents a hydrocarbon group having 1 to 16 carbon atoms.

R¹¹ preferably represents a hydrocarbon group having 1 to 16 carbon atoms and more preferably represents a hydrocarbon group having 1 to 9 carbon atoms.

R¹² preferably represents a hydrocarbon group having 1 to 20 carbon atoms and more preferably represents a hydrocarbon group having 1 to 16 carbon atoms.

The hydrocarbon groups represented by R⁹ and R¹² may be preferably substituted with an aryl group or —S—R⁵⁸. Here, R⁵⁸ preferably represents a hydrocarbon group having 1 to 8 carbon atoms.

The hydrocarbon group represented by R¹¹ may be preferably substituted with —C(O)O—R⁵⁵ or —OC(O)—R⁵⁶, where R⁵⁵ and R⁵⁶ each independently represent a hydrocarbon group having 1 to 16 carbon atoms.

The hydrocarbon groups represented by R⁵⁵ and R⁵⁶ may be preferably substituted with an aryl group having 6 to 20 carbon atoms or —S—R⁵⁸, and the definition of R⁵⁸ is as described above.

The compound represented by Formula (1) is preferably a compound represented by Formula (1-1) as a first example.

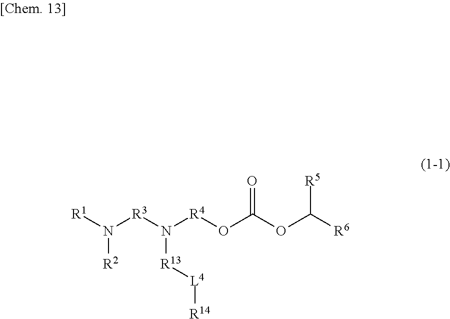

In the formula,
R¹ and R² each independently represent a hydrocarbon group having 1 to 18 carbon atoms, and R³ represents a hydrocarbon group having 2 to 8 carbon atoms, where the hydrocarbon groups represented by R¹, R², and R³ may be substituted with —OH, COOH, —NR⁵¹R⁵², —OC(O)O—R⁵³, —C(O)O—R⁵⁴, —OC(O)—R⁵⁵, or —O—R⁵⁶,
R⁴ represents a hydrocarbon group having 1 to 8 carbon atoms,
R⁵ and R⁶ each independently represent a hydrocarbon group having 1 to 8 carbon atoms or —R⁸-L¹-R⁹, excluding a case that both R⁵ and R⁶ are hydrocarbon groups having 1 to 8 carbon atoms,
L¹ represents —OC(O)O—, —C(O)O—, —OC(O)—, or —O—,
R⁸ represents a hydrocarbon group having 1 to 12 carbon atoms,
R⁹ represents a hydrocarbon group having 1 to 24 carbon atoms, where the hydrocarbon group represented by R⁹ may be substituted with an aryl group, —OC(O)O—R⁵³, —C(O)O—R⁵⁴, —OC(O)—R⁵⁵, or —S—R⁵⁸,
R⁵¹ and R⁵² each independently represent a hydrocarbon group having 1 to 8 carbon atoms,
R⁵³, R⁵⁴, R⁵⁵, and R⁵⁶ each independently represent a hydrocarbon group having 1 to 24 carbon atoms,
the hydrocarbon groups represented by R⁵³, R⁵⁴, R⁵⁵, and R⁵⁶ may be substituted with an aryl group having 6 to 20 carbon atoms or —S—R⁵⁸,
the above-described aryl group having 6 to 20 carbon atoms may be substituted with —OH, COOH, —NR⁵¹R⁵², —OC(O)O—R⁵³, —C(O)O—R⁵⁴, —OC(O)—R⁵⁵, —O—R⁵⁶, or -(hydrocarbon group having 1 to 12 carbon atoms)-R⁵⁷,
R⁵⁸ represents a hydrocarbon group having 1 to 12 carbon atoms, and
R⁵⁷ represents —OH, COOH, —NR⁵¹R⁵², —OC(O)O—R⁵³, —C(O)O—R⁵⁴, —OC(O)—R⁵⁵, or —O—R⁵⁶.
R¹³ represents a hydrocarbon group having 1 to 8 carbon atoms,
R¹⁴ represents —R¹⁵-L⁵-R¹⁶, where R¹⁵ represents a hydrocarbon group having 1 to 24 carbon atoms, L⁵ represents —OC(O)O—, —C(O)O—, —OC(O)—, or —O—, and R¹⁶ represents a hydrocarbon group having 1 to 24 carbon atoms,
the hydrocarbon group having 1 to 24 carbon atoms represented by R¹⁵ may be substituted with —OC(O)O—R⁵³, —C(O)O—R⁵⁴, or —OC(O)—R⁵⁵, where definitions of R⁵³, R⁵⁴, and R⁵⁵ are as described above, and
the hydrocarbon group having 1 to 24 carbon atoms represented by R¹⁶ may be substituted with an aryl group having 6 to 20 carbon atoms, —OC(O)O—R⁵³, —C(O)O—R⁵⁴, —OC(O)—R⁵⁵ or —S—R⁵⁸, where the definitions of R⁵³, R⁵⁴, R⁵⁵, and R⁵⁸ are as described above.

In Formula (1-1), R¹ and R² each independently preferably represent a hydrocarbon group having 1 to 12 carbon atoms, more preferably represent a hydrocarbon group having 1 to 6 carbon atoms, and even more preferably represent a hydrocarbon group having 1 to 3 carbon atoms.

R³ preferably represents a hydrocarbon group having 2 to 6 carbon atoms and more preferably represents a hydrocarbon group having 2 to 4 carbon atoms.

The hydrocarbon groups represented by R¹, R², and R³ may be preferably substituted with —OH.

L¹ preferably represents —C(O)O— or —OC(O)—.
R⁸ preferably represents a hydrocarbon group having 1 to 10 carbon atoms and more preferably represents a hydrocarbon group having 1 to 8 carbon atoms.

R⁹ preferably represents a hydrocarbon group having 1 to 18 carbon atoms, and the hydrocarbon group represented by R⁹ may be substituted with an aryl group having 6 to 20 carbon atoms or —S—R⁵⁸.

R¹⁴ preferably represents —R¹⁵-L⁵-R¹⁶, where R¹⁵ represents a hydrocarbon group having 1 to 18 carbon atoms, L⁵ represents —OC(O)O—, and R¹⁶ represents a hydrocarbon group having 1 to 18 carbon atoms.

The hydrocarbon group having 1 to 18 carbon atoms represented by R¹⁵ may be preferably substituted with —C(O)O—R⁵⁵ or —OC(O)—R⁵⁶. R⁵⁵ and R⁵⁶ each independently represent a hydrocarbon group having 1 to 16 carbon atoms, and the hydrocarbon groups represented by R⁵⁵ and R⁵⁶ may be substituted with an aryl group having 6 to 20 carbon atoms or —S—R⁵⁸, where the definition of R⁵⁸ is as described above.

The hydrocarbon group having 1 to 18 carbon atoms represented by R¹⁶ may be preferably substituted with an aryl group or —S—R⁵⁸, where the definition of R⁵⁸ is as described above.

The compound represented by Formula (1) is preferably a compound represented by Formula (1-2) as a second example.

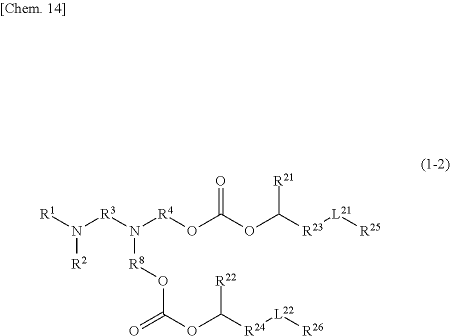

In the formula,
R¹ and R² each independently represent a hydrocarbon group having 1 to 18 carbon atoms, and R³ represents a hydrocarbon group having 2 to 8 carbon atoms, where the hydrocarbon groups represented by R¹, R², and R³ may be substituted with —OH, COOH, —NR⁵¹R⁵², —OC(O)O—R⁵³, —C(O)O—R⁵⁴, —OC(O)—R⁵⁵, or —O—R⁵⁶,
R⁴ and R⁸ each independently represent a hydrocarbon having 1 to 8 carbon atoms,
R²¹ and R²² each independently represent a hydrocarbon group having 1 to 18 carbon atoms,
R²³ and R²⁴ each independently represent a hydrocarbon group having 1 to 12 carbon atoms,
R²⁵ and R²⁶ each independently represent a hydrocarbon group having 1 to 24 carbon atoms,
L²¹ and L²² each independently represent —OC(O)O—, —C(O)O—, —OC(O)—, or —O—,
the hydrocarbon groups represented by R²⁵ and R²⁶ may be substituted with an aryl group having 6 to 20 carbon atoms, —OC(O)O—R⁵³, —C(O)O—R⁵⁴, —OC(O)—R⁵⁵, or —S—R⁵⁸,
R⁵¹ and R⁵² each independently represent a hydrocarbon group having 1 to 8 carbon atoms,
R⁵³, R⁵⁴, R⁵⁵, and R⁵⁶ each independently represent a hydrocarbon group having 1 to 18 carbon atoms,
the above-described aryl group having 6 to 20 carbon atoms may be substituted with OH, COOH, —NR⁵¹R⁵², —OC(O)O—R⁵³, —C(O)O—R⁵⁴, —OC(O)—R⁵⁵, —O—R⁵⁶, or -(hydrocarbon group having 1 to 12 carbon atoms)-R⁵⁷, and
R⁵⁷ represents —OH, COOH, —NR⁵¹R⁵², —OC(O)O—R⁵³, —C(O)O—R⁵⁴, —OC(O)—R⁵⁵, or —O—R⁵⁶.
R⁵⁸ represents a hydrocarbon group having 1 to 12 carbon atoms.

In Formula (1-2), R¹ and R² each independently preferably represent a hydrocarbon group having 1 to 12 carbon atoms, more preferably represent a hydrocarbon group having 1 to 6 carbon atoms, and even more preferably represent a hydrocarbon group having 1 to 3 carbon atoms. The hydrocarbon groups represented by R¹ and R² may be preferably substituted with —OH, but has more preferably a hydrocarbon having no substituent.

R³ preferably represents a hydrocarbon group having 2 to 6 carbon atoms and more preferably represents a hydrocarbon group having 2 to 4 carbon atoms.

R²¹ and R²² each independently preferably represent a hydrocarbon group having 1 to 12 carbon atoms, more preferably represent a hydrocarbon group having 1 to 8 carbon atoms, and even more preferably represent a hydrocarbon group having 1 to 6 carbon atoms.

R²³ and R²⁴ each independently preferably represent a hydrocarbon group having 1 to 10 carbon atoms and more preferably represent a hydrocarbon group having 1 to 8 carbon atoms

R²⁵ and R²⁶ each independently preferably represent a hydrocarbon group having 1 to 20 carbon atoms, more preferably represent a hydrocarbon group having 1 to 16 carbon atoms, and even more preferably represent a hydrocarbon group having 1 to 12 carbon atoms.

L²¹ and L²² each independently preferably represent —C(O)O— or —OC(O)—.

The compound represented by Formula (1) is preferably a compound represented by Formula (1-3) as a third example.

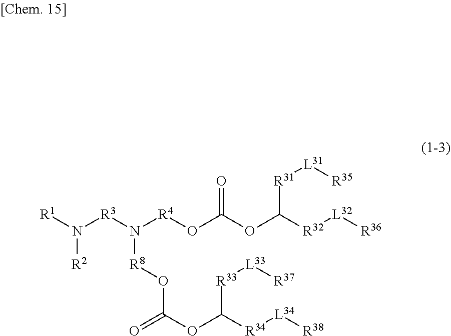

In the formula,
R¹ and R² each independently represent a hydrocarbon group having 1 to 18 carbon atoms, and R³ represents a hydrocarbon group having 2 to 8 carbon atoms, where the hydrocarbon groups represented by R¹, R², and R³ may be substituted with —OH, COOH, —NR⁵¹R⁵², —OC(O)O—R⁵³, —C(O)O—R⁵⁴, —OC(O)—R⁵⁵, or —O—R⁵⁶,
R⁴ and R⁸ each independently represent a hydrocarbon group having 1 to 8 carbon atoms,
R³¹, R³², R³³, and R³⁴ each independently represent a hydrocarbon group having 1 to 12 carbon atoms,
R³⁵, R³⁶, R³⁷, and R³⁸ each independently represent a hydrocarbon group having 1 to 24 carbon atoms,
L³¹, L³², L³³, and L³⁴ each independently represent —OC(O)O—, —C(O)O—, —OC(O)—, or —O—,
the hydrocarbon groups represented by R³⁵, R³⁶, R³⁷, and R³⁸ may be substituted with an aryl group having 6 to 20 carbon atoms, —OC(O)O—R⁵³, —C(O)O—R⁵⁴, —OC(O)—R⁵⁵, or S—R⁵⁸,
R⁵¹ and R⁵² each independently represent a hydrocarbon group having 1 to 8 carbon atoms,
R⁵³, R⁵⁴, R⁵⁵, and R⁵⁶ each independently represent a hydrocarbon group having 1 to 18 carbon atoms,
the above-described aryl group having 6 to 20 carbon atoms may be substituted with OH, COOH, —NR⁵¹R⁵², —OC(O)O—R⁵³, —C(O)O—R⁵⁴, —OC(O)—R⁵⁵, —O—R⁵⁶, or -(hydrocarbon group having 1 to 12 carbon atoms)-R⁵⁷, and
R⁵⁷ represents —OH, COOH, —NR⁵¹R⁵², —OC(O)O—R⁵³, —C(O)O—R⁵⁴, —OC(O)—R⁵⁵, or —O—R⁵⁶.
R⁵⁸ represents a hydrocarbon group having 1 to 12 carbon atoms.

In Formula (1-3), R¹ and R² each independently preferably represent a hydrocarbon group having 1 to 12 carbon atoms, more preferably represent a hydrocarbon group having 1 to 6 carbon atoms, and even more preferably represent a hydrocarbon group having 1 to 3 carbon atoms. The hydrocarbon groups represented by R¹ and R² may be preferably substituted with —OH, but has more preferably a hydrocarbon having no substituent.

R³ preferably represents a hydrocarbon group having 2 to 6 carbon atoms and more preferably represents a hydrocarbon group having 2 to 4 carbon atoms.

R³¹, R³², R³³, and R³⁴ each independently preferably represent a hydrocarbon group having 1 to 10 carbon atoms, more preferably represent a hydrocarbon group having 1 to 8 carbon atoms, and even more preferably represent a hydrocarbon group having 1 to 3 carbon atoms.

R³⁵, R³⁶, R³⁷, and R³⁸ each independently preferably represent a hydrocarbon group having 1 to 20 carbon atoms, more preferably represent a hydrocarbon group having 1 to 16 carbon atoms, and even more preferably represent a hydrocarbon group having 1 to 12 carbon atoms. The hydrocarbon groups represented by R³⁵, R³⁶, R³⁷, and R³⁸ may be preferably substituted with an aryl group having 6 to 20 carbon atoms or S—R⁵⁸. More preferably, these may be substituted with —S—R⁵⁸.

R³⁵, R³⁶, R³⁷, and R³⁸ each independently particularly preferably represent a hydrocarbon group having 1 to 12 carbon atoms substituted with —S—R⁵⁸, or a hydrocarbon group having 1 to 12 carbon atoms.

L³¹, L³², L³³, and L³⁴ each independently preferably represent —C(O)O—, or —OC(O)—.

R⁵⁸ preferably represents a hydrocarbon group having 1 to 10 carbon atoms and more preferably represents a hydrocarbon group having 1 to 8 carbon atoms.

The compound according to the embodiment of the present invention may form a salt.

Examples of the salt in a basic group include salts with mineral acids such as hydrochloric acid, hydrobromic acid, nitric acid, and sulfuric acid; salts with organic acid, succinic acid, malic acid, tartaric acid, aspartic acid, trichloroacetic acid, and trifluoroacetic acid; and salts with sulfonic acids such as methanesulfonic acid, benzenesulfonic acid, p-toluenesulfonic acid, mesitylenesulfonic acid, and naphthalenesulfonic acid.

Examples of the salt in an acidic group include salts with alkali metals such as sodium and potassium; salts with alkaline earth metals such as calcium and magnesium; ammonium salts; salts with nitrogen-containing organic bases such as trimethylamine, triethylamine, tributylamine, pyridine, N,N-dimethylaniline, N-methylpiperidine, N-methylmorpholine, diethylamine, dicyclohexylamine, procaine, dibenzylamine, N-benzyl-β-phenethylamine, 1-ephenamine, and N,N′-dibenzylethylenediamine; and the like.

Among the above-described salts, for example, pharmacologically acceptable salts are preferable.

The lipid represented by the formula (1) and a method for producing the same are described in WO2022/230964A, the entire of which is incorporated herein by reference.

### Lipid Represented by the Formula (5) or a Salt Thereof

For example, a lipid represented by Formula (5) or a salt thereof may be used as the ionizable lipid.

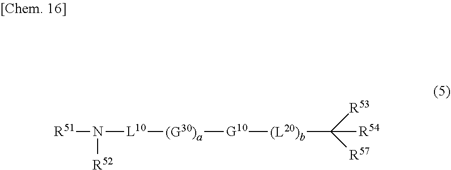

wherein R⁵¹ and R⁵² each independently represent a hydrocarbon group having 1 to 21 carbon atoms which may have a substituent A,
the substituent A represents a hydroxyl group, or a group represented by -G²⁰-CH(R⁵⁵)(R⁵⁶), —N(R⁵⁸)(R⁵⁹) or -G²⁰-R⁶⁰,
G²⁰ represents —O(CO)—, or —(CO)O—,
R⁵⁵ and R⁵⁶ each independently represent a hydrogen atom or a hydrocarbon group having 1 to 18 carbon atoms,
R⁵⁸ and R⁵⁹ each independently represent a hydrogen atom or a cyclic hydrocarbon group having 3 to 6 carbon atoms which may have a substituent B,
the substituent B is —N(R⁶¹)(R⁶²),
R⁶¹ and R⁶² each independently represent a hydrogen atom or a hydrocarbon group having 1 to 6 carbon atoms,
R⁶⁰ represents a hydrocarbon group having 1 to 18 carbon atoms,
L¹⁰ represents a hydrocarbon group having 1 to 18 carbon atoms,
G³⁰ indicates —S—(CO)—NR⁶⁴,
R⁶⁴ represents a group represented by -L³⁰-G²⁰-CH(R⁵⁵)(R⁵⁶),
a represents 0 or 1,
L³⁰ represents a single bond or a hydrocarbon group having 1 to 18 carbon atoms,
G¹⁰ represents —O(CO)—, —(CO)O—, —O(CO)O—, or —N(C(O)R⁶³)—,
R⁶³ represents a hydrocarbon group having 1 to 18 carbon atoms,
L²⁰ represents a hydrocarbon group having 1 to 6 carbon atoms,
b represents 0 or 1,
R⁵³, R⁵⁴ and R⁵⁷ each independently represent a hydrogen atom or a hydrocarbon group having 1 to 21 carbon atoms which may have a substituent C,
the substituent C represents a group represented by —(CO)OR⁶⁵ or —O(CO)—R⁶⁵,
R⁶⁵ represents a hydrocarbon group having 1 to 18 carbon atoms or a group represented by -L⁴⁰-CH(R⁶⁶)(R⁶⁷),
L⁴⁰ represents a hydrocarbon group having 1 to 6 carbon atoms,
R⁶⁶ and R⁶⁷ represent a hydrocarbon group having 1 to 10 carbon atoms or an alkoxy group.

The compound represented by Formula (5) may be a compound represented by Formula (5A):

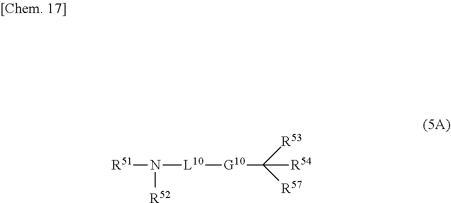

wherein R⁵¹ and R⁵² each independently represent a hydrocarbon group having 1 to 21 carbon atoms which may have a substituent A,
the substituent A represents a hydroxyl group, or a group represented by -G²⁰-CH(R⁵⁵)(R⁵⁶),
G²⁰ represents —O(CO)—, or —(CO)O—,
R⁵⁵ and R⁵⁶ each independently represent a hydrocarbon group having 1 to 18 carbon atoms,
L¹⁰ represents a hydrocarbon group having 1 to 18 carbon atoms,
G¹⁰ represents —O(CO)—, or —(CO)O—,
R⁶³ represents a hydrocarbon group having 1 to 18 carbon atoms,
R⁵³, R⁵⁴ and R⁵⁷ each independently represent a hydrogen atom or a hydrocarbon group having 1 to 21 carbon atoms.

The compound represented by Formula (5) may be a compound represented by Formula (5B):

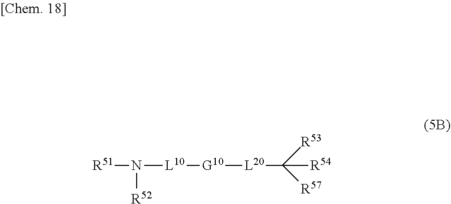

wherein R⁵¹ and R⁵² each independently represent a hydrocarbon group having 1 to 21 carbon atoms,
L¹⁰ represents a hydrocarbon group having 1 to 18 carbon atoms,
G¹⁰ represents —O(CO)O—,
L²⁰ represents a hydrocarbon group having 1 to 6 carbon atoms,
R⁵³, R⁵⁴ and R⁵⁷ each independently represent a hydrogen atom or a hydrocarbon group having 1 to 21 carbon atoms which may have a substituent C,
the substituent C represents a group represented by —O(CO)—R⁶⁵,
R⁶⁵ represents a hydrocarbon group having 1 to 18 carbon atoms or a group represented by -L⁴⁰-CH(R⁶⁶)(R⁶⁷),
L⁴⁰ represents a hydrocarbon group having 1 to 6 carbon atoms,
R⁶⁶ and R⁶⁷ represent an alkoxy group having 1 to 10 carbon atoms.

The compound represented by Formula (5) may be a compound represented by Formula (5C):

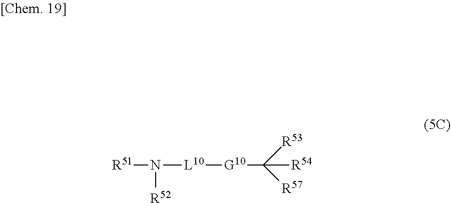

wherein R⁵¹ and R⁵² each independently represent a hydrocarbon group having 1 to 21 carbon atoms
L¹⁰ represents a hydrocarbon group having 1 to 18 carbon atoms,
G¹⁰ represents —N(C(O)R⁶³)—,
R⁶³ represents a hydrocarbon group having 1 to 18 carbon atoms,
R⁵³, R⁵⁴ and R⁵⁷ each independently represent a hydrogen atom or a hydrocarbon group having 1 to 21 carbon atoms which may have a substituent C,
the substituent C represents a group represented by —(CO)OR⁶⁵,
R⁶⁵ represents a group represented by -L⁴⁰-CH(R⁶⁶)(R⁶⁷),
L⁴⁰ represents a hydrocarbon group having 1 to 6 carbon atoms,
R⁶⁶ and R⁶⁷ represent a hydrocarbon group having 1 to 10 carbon atoms.

The compound represented by Formula (5) may be a compound represented by Formula (5D):

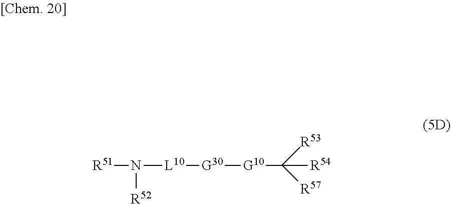

wherein R⁵¹ and R⁵² each independently represent a hydrocarbon group having 1 to 21 carbon atoms,
L¹⁰ represents a hydrocarbon group having 1 to 18 carbon atoms,
G³⁰ indicates —S—(CO)—NR⁶⁴,
R⁶⁴ represents a group represented by -L³⁰-G²⁰-CH(R⁵⁵)(R⁵⁶),
L³⁰ represents a single bond or a hydrocarbon group having 1 to 18 carbon atoms,
G²⁰ represents —(CO)O—,
R⁵⁵ and R⁵⁶ each independently represent a hydrogen atom or a hydrocarbon group having 1 to 18 carbon atoms,
G¹⁰ represents —(CO)O—,
R⁵³, R⁵⁴ and R⁵⁷ each independently represent a hydrogen atom or a hydrocarbon group having 1 to 21 carbon atoms.

The hydrocarbon group having 1 to 21 carbon atoms is preferably an alkyl group having 1 to 21 carbon atoms, an alkenyl group having 2 to 21 carbon atoms, or an alkynyl group having 2 to 21 carbon atoms, more preferably an alkyl group having 1 to 21 carbon atoms, or an alkenyl group having 2 to 21 carbon atoms. The alkyl group having 1 to 21 carbon atoms may be linear or branched, and may be chain or cyclic. The number of carbon atoms is preferably 3 to 21, and more preferably 5 to 21 carbon atoms. Examples include propyl group, isopropyl group, cyclopropyl group, butyl group, isobutyl group, tert-butyl group, cyclobutyl group, pentyl group, cyclopentyl group, hexyl group, cyclohexyl group, heptyl group, octyl group, nonyl group, decyl group, undecyl group, dodecyl group, tridecyl group, trimethyldodecyl group (preferably a 3,7,11-trimethyldodecyl group), tetradecyl group, pentadecyl group, hexadecyl group, heptadecyl group and octadecyl group. The alkenyl group having 2 to 18 carbon atoms may be linear or branched, and may be chain or cyclic. The number of carbon atoms is preferably 3 to 18, and more preferably 5 to 18. Examples include allyl group, prenyl group, pentanyl group, hexenyl group, heptenyl group, octenyl group, nonenyl group (preferably (Z)-2-nonenyl group or (E)-2-nonenyl group), decenyl group, undecenyl group, dodecenyl group, dodecadienyl group, tridecenyl group (preferably (Z)-trideca-8-enyl group), tetradecenyl group (preferably tetradeca-9-enyl group), pentadecenyl group (preferably (Z)-pentadeca-8-enyl group), hexadecenyl group (preferably (Z)-hexadeca-9-enyl group), hexadecadienyl group, heptadecenyl group (preferably (Z)-heptadeca-8-enyl group), heptadecadienyl group (preferably (8Z, 11Z)-heptadeca-8,11-dienyl group), octadecenyl group (preferably (Z)-octadeca-9-enyl group), octadecadienyl Groups (preferably (9Z, 12Z)-octadeca-9,12-dienyl group). The alkynyl group having 2 to 21 carbon atoms may be linear or branched, and may be chain or cyclic. The number of carbon atoms is preferably 3 to 21, and more preferably 5 to 21 carbon atoms. Examples include propargyl group, butynyl group, pentynyl group, hexynyl group, heptynyl group, octynyl group, nonynyl group, decynyl group, undecynyl group, dodecynyl group, tetradecynyl group, pentadecynyl group, hexadecynyl group, heptadecynyl group, octadecynyl group and the like. Examples of the hydrocarbon group having 1 to 18 carbon atoms include those having 1 to 18 carbon atoms among the hydrocarbon groups having 1 to 21 carbon atoms.

As the cyclic hydrocarbon group, a cycloalkyl group having 3 to 10 carbon atoms, a cycloalkenyl group having 3 to 10 carbon atoms, a cycloalkynyl group having 3 to 10 carbon atoms, and an aryl group having 6 to 10 carbon atoms are preferable.

The hydrocarbon group having 1 to 6 carbon atoms is preferably an alkyl group having 1 to 6 carbon atoms, an alkenyl group having 2 to 6 carbon atoms or an alkynyl group having 2 to 6 carbon atoms, and more preferably an alkyl group having 1 to 6 carbon atoms or an alkenyl group having 2 to 6 carbon atoms. The alkyl group having 1 to 6 carbon atoms may be linear or branched, and may be chain or cyclic. Specific examples thereof include propyl group, isopropyl group, cyclopropyl group, butyl group, isobutyl group, tert-butyl group, cyclobutyl group, pentyl group, cyclopentyl group and hexyl group. The alkenyl group having 2 to 6 carbon atoms may be linear or branched, and may be chain or cyclic. Specific examples thereof include allyl group, prenyl group, pentenyl group, and hexenyl group. The alkynyl group having 2 to 6 carbon atoms may be linear or branched, and may be chain or cyclic. Specific examples thereof include propargyl group, butynyl group, pentynyl group, and hexynyl group.

The hydrocarbon group having 1 to 10 carbon atoms is preferably an alkyl group having 1 to 10 carbon atoms, an alkenyl group having 2 to 10 carbon atoms, or an alkynyl group having 2 to 10 carbon atoms, and preferably an alkyl group having 1 to 10 carbon atoms or an alkenyl group having 2 to 10 carbon atoms. The alkyl group having 1 to 10 carbon atoms may be linear or branched, and may be chain or cyclic. The number of carbon atoms is preferably 3 to 10, and more preferably 5 to 10 carbon atoms. Specific example include propyl group, isopropyl group, cyclopropyl group, butyl group, isobutyl group, tert-butyl group, cyclobutyl group, pentyl group, cyclopentyl group, hexyl group, cyclohexyl group, heptyl group, octyl group, nonyl group, and decyl group. The alkenyl group having 2 to 10 carbon atoms may be linear or branched, and may be chain or cyclic. The number of carbon atoms is preferably 3 to 10, more preferably 5 to 10. Specific examples include allyl group, prenyl group, pentenyl group, hexenyl group, heptenyl group, octenyl group, a nonenyl group (preferably (Z)-2-nonenyl group or (E)-2-nonenyl group), and decenyl group. The alkynyl group having 2 to 10 carbon atoms may be linear or branched, and may be chain or cyclic. The number of carbon atoms is preferably 3 to 10, and more preferably 5 to 10 carbon atoms. Specific examples thereof include propargyl group, butynyl group, pentynyl group, hexynyl group, heptynyl group, octynyl group, noninyl group and a decynyl group.

The compound represented by Formula (5) may form a salt.

Examples of the salt in a basic group include salts with mineral acids such as hydrochloric acid, hydrobromic acid, nitric acid, and sulfuric acid; salts with organic carboxylic acids such as formic acid, acetic acid, citric acid, oxalic acid, fumaric acid, maleic acid, succinic acid, malic acid, tartaric acid, aspartic acid, trichloroacetic acid, and trifluoroacetic acid; and salts with sulfonic acids such as methanesulfonic acid, benzenesulfonic acid, p-toluenesulfonic acid, mesitylenesulfonic acid, and naphthalenesulfonic acid.

Among the above salts, for example, pharmacologically acceptable salts are preferable.

The lipid represented by the formula (4) and a method for producing the same are described in WO2019/235635A and WO2021/095876A.

### Examples of Ionizable Lipids

Examples of ionizable lipids include the following lipids. Note that cKK-E12 (MD-1), C12-200, 306Oi10, YSK05, and 93-O17S are compounds which is not included in the above formula (5).

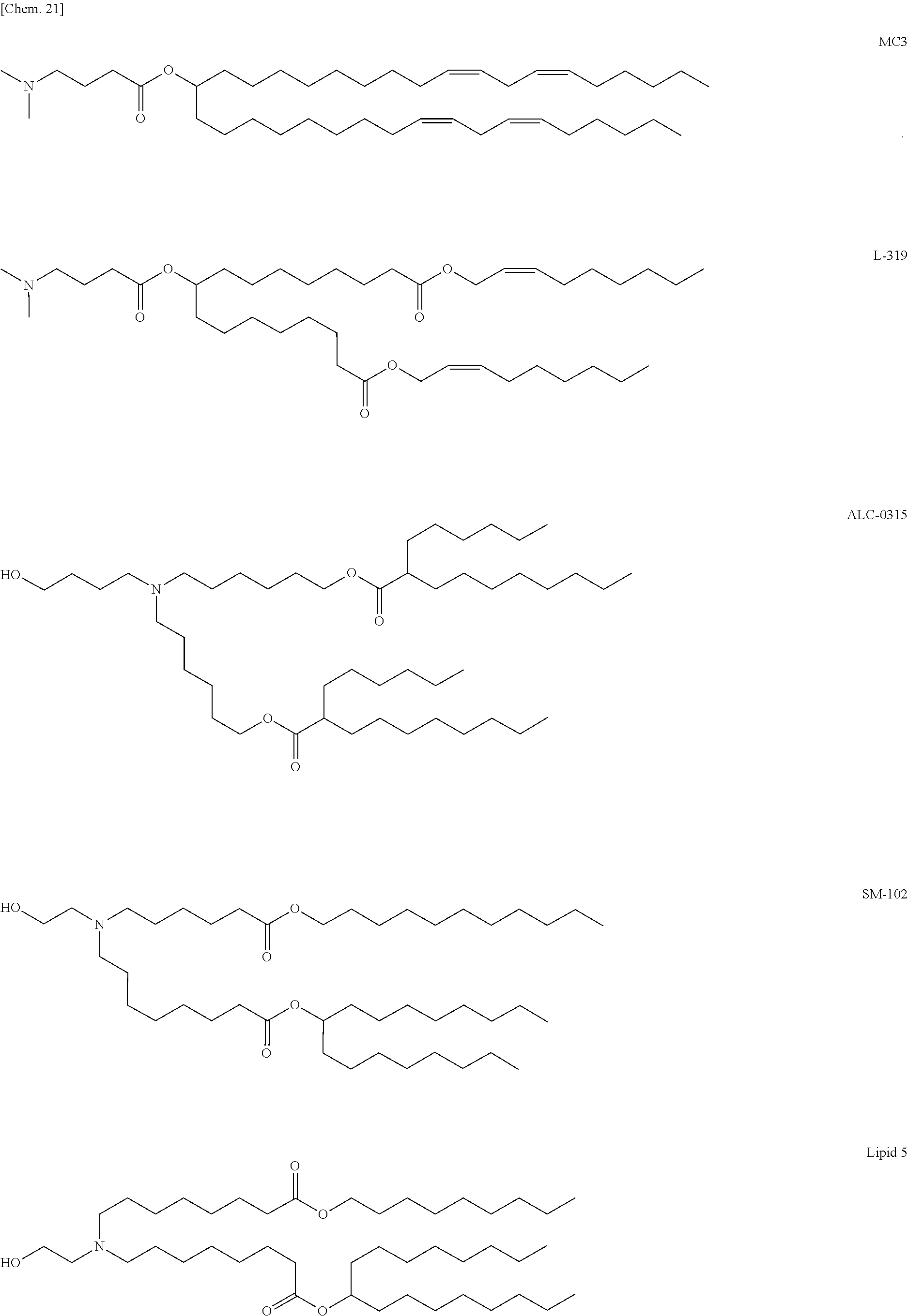

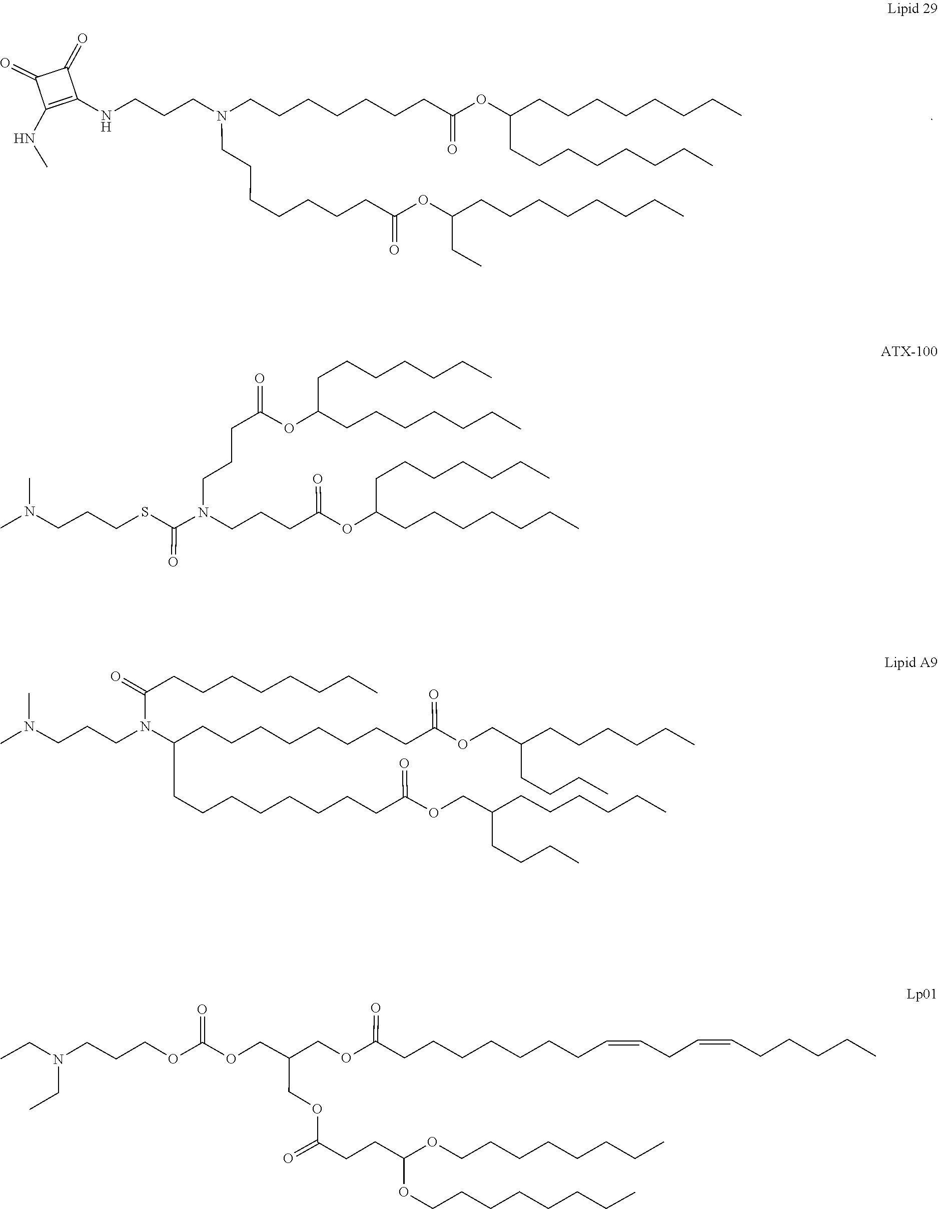

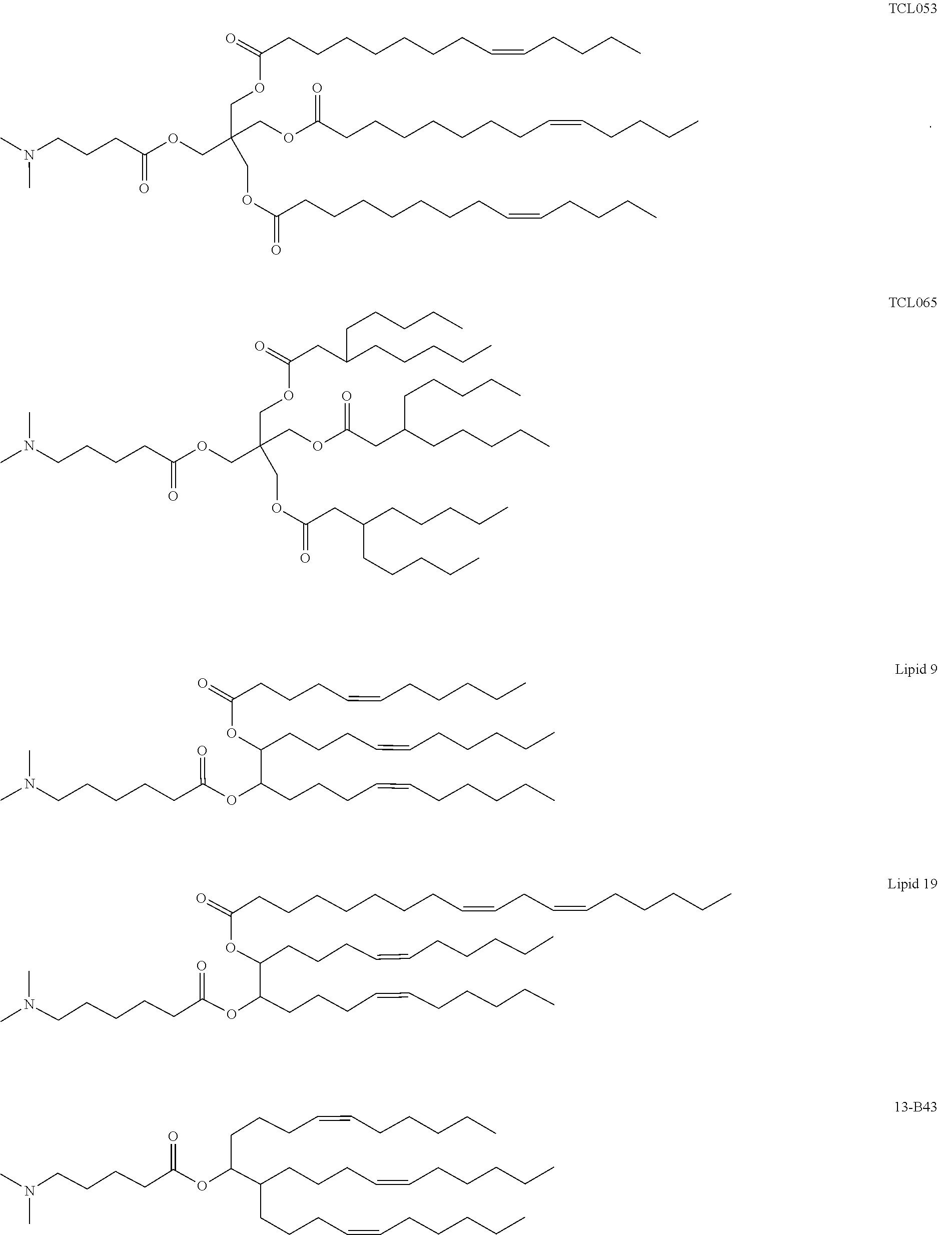

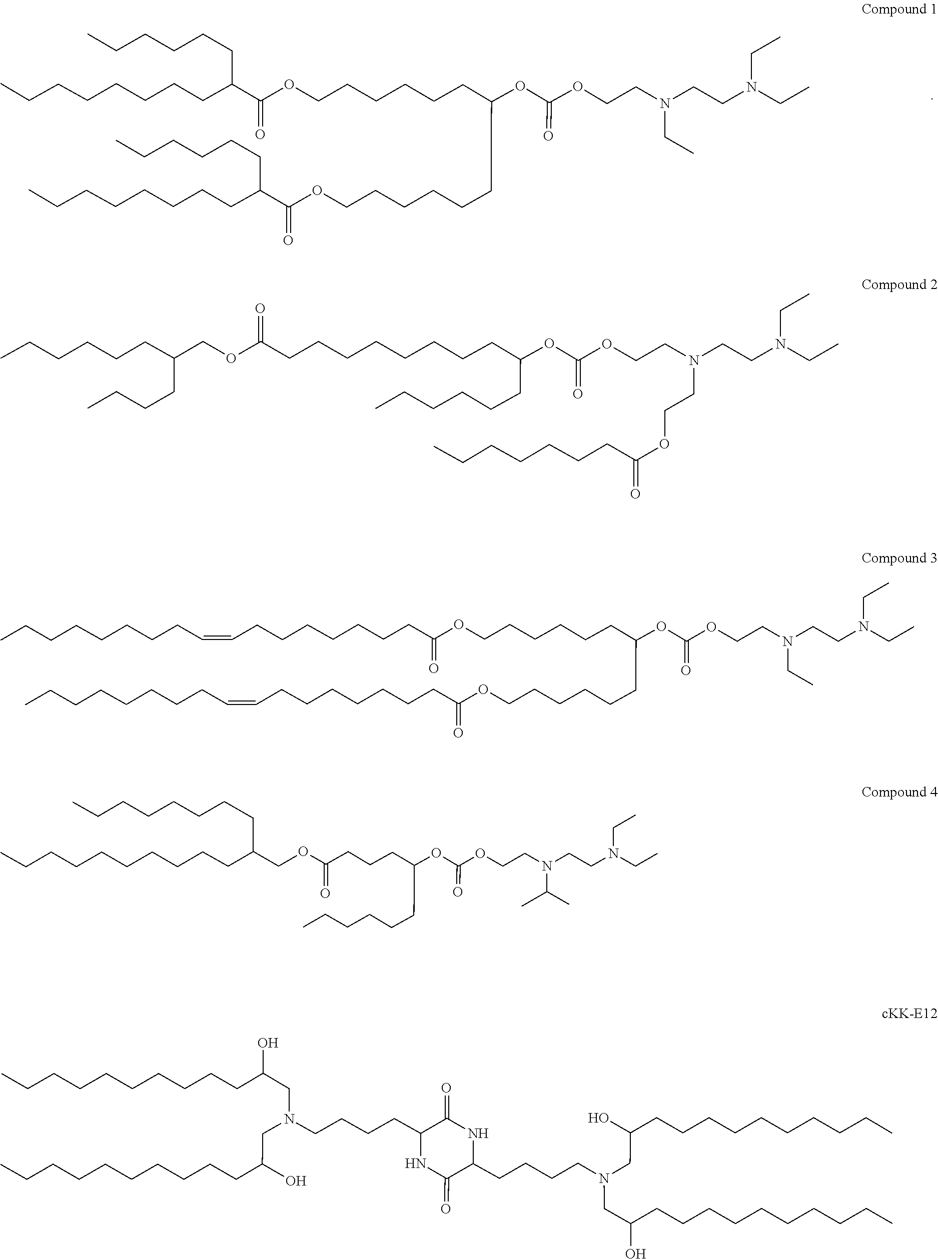

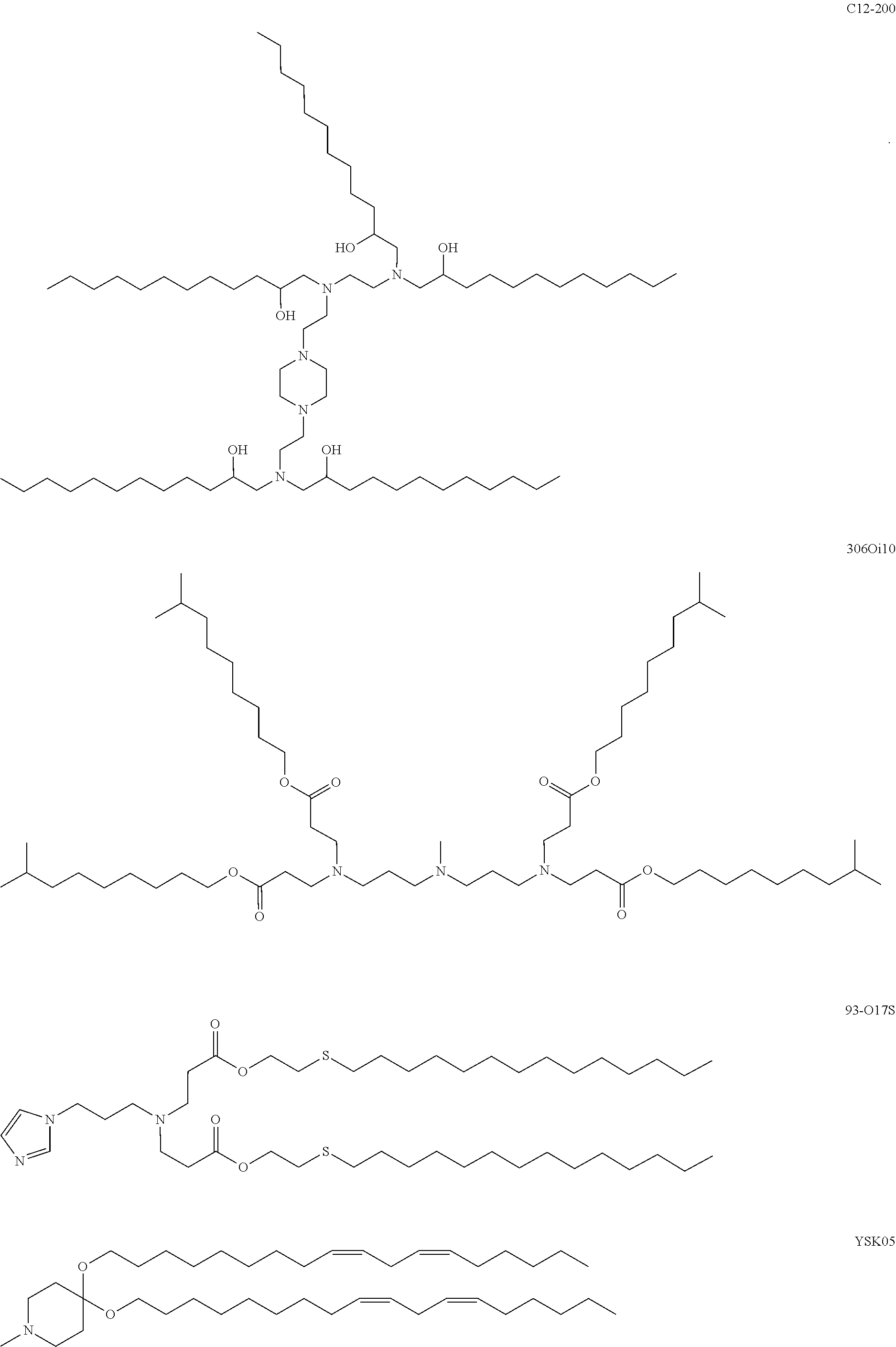

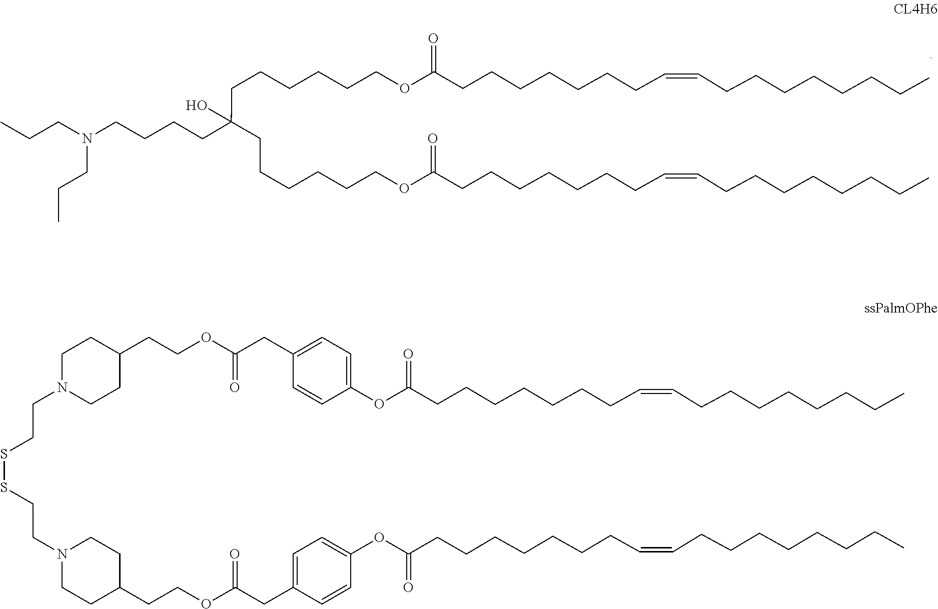 [SMILES: CCCCCCCC/C=C\CCCCCCCC(=O)OCCCCCCC(O)(CCCCCCOC(=O)CCCCCCC/C=C\CCCCCCCC)CCCCN(CCC)CCC ; CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1]

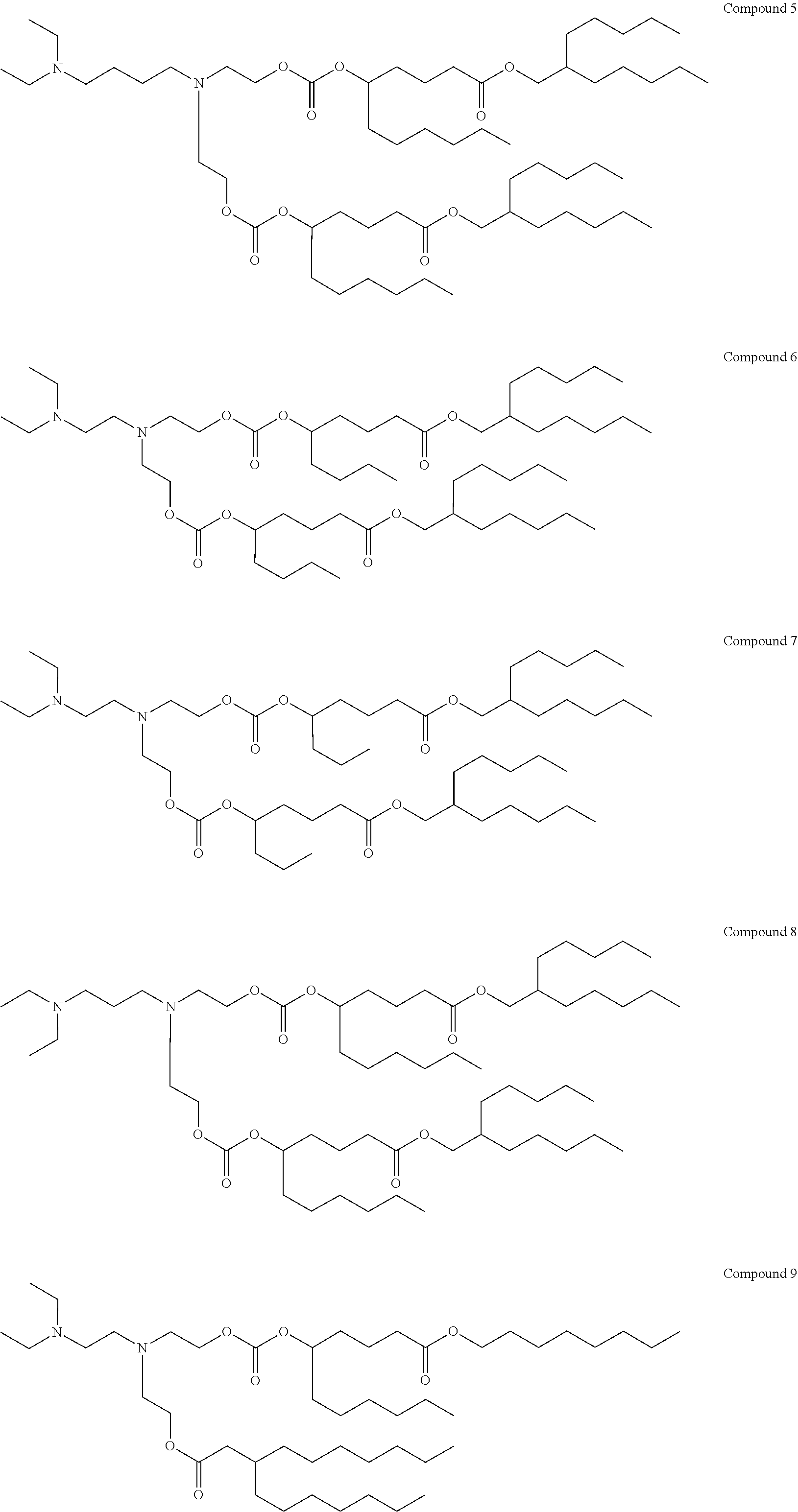

In the lipid composition of the present invention, the content of the ionizable lipid or a salt thereof with respect to the total lipids is preferably 10 mol % to 80 mol %, more preferably 20 mol % to 80 mol %, still more preferably 30 mol % to 70 mol %, further more preferably 40 mol % to 60 mol %.

### Neutral Lipid

The lipid particles according of the present invention may contain a neutral lipid.

The neutral lipid is preferably Zwitterionic lipid.

As the zwitterionic lipid, phospholipid is preferable. Examples thereof include phosphatidylcholine, phosphatidylethanolamine, sphingomyelin, and the like. As the phospholipid, a phospholipid having a choline group such as phosphatidylcholine is preferable. The zwitterionic lipid may be used alone or in combination of a plurality of different neutral lipids.

The phosphatidylcholine is not particularly limited, and examples thereof include soybean lecithin (SPC), hydrogenated soybean lecithin (HSPC), egg yolk lecithin (EPC), hydrogenated egg yolk lecithin (HEPC), dimyristoylphosphatidylcholine (DMPC), dipalmitoylphosphatidylcholine (DPPC), distearoylphosphatidylcholine (DSPC), dioleoylphosphatidylcholine (DOPC), dilauroylphosphatidylcholine (DLPC), 1-palmitoyl-2-oleoylphosphatidylcholine (POPC), and the like. Among these, dimyristoylphosphatidylcholine (DMPC), distearoylphosphatidylcholine (DSPC) and dilauroylphosphatidylcholine (DLPC) are preferable. Particularly, distearoylphosphatidylcholine (DSPC) is preferable.
DSPC: 1,2-Distearoyl-sn-glycero-3-phosphocholine

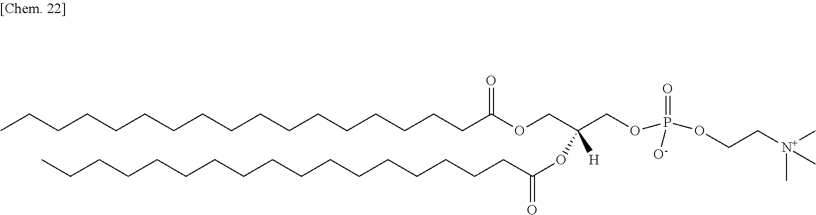 [SMILES: CCCCCCCCCCCCCCCCCC(=O)OC[C@H](COP(=O)([O-])OCC[N+](C)(C)C)OC(=O)CCCCCCCCCCCCCCCCC]

The phosphatidylethanolamine is not particularly limited, and examples thereof include dimyristoylphosphatidylethanolamine (DMPE), dipalmitoylphosphatidylethanolamine (DPPE), distearoylphosphatidylethanolamine (DSPE), dioleoylphosphatidylethanolamine (DOPE), dilinoleoylphosphatidylethanolamine (DLoPE), diphytanoylphosphatidylethanolamine (D(Phy)PE), 1-palmitoyl-2-oleoylphosphatidylethanolamine (POPE), ditetradecylphosphatidylethanolamine, dihexadecylphosphatidylethanolamine, dioctadecylphosphatidylethanolamine, diphytanylphosphatidylethanolamine, and the like.

The sphingomyelin is not particularly limited, and examples thereof include egg yolk-derived sphingomyelin, milk-derived sphingomyelin, and the like.

In the lipid composition of the present invention, the amount of the neutral lipid mixed in is preferably 1 to 30 mol %, more preferably 5 to 25 mol %, still more preferably 7 to 23 mol % with respect to the total amount of the constituent lipid components.

### Lipid Having Nonionic Hydrophilic Polymer

The lipid composition of the present invention may contain a lipid having a nonionic hydrophilic polymer. The lipid having nonionic hydrophilic polymer preferably contains an acyl group, and the carbon chain length of the acyl group is preferably 8 to 26.

The nonionic hydrophilic polymer is not particularly limited, and examples thereof include a nonionic vinyl-based polymer, a nonionic polyamino acid, a nonionic polyester, a nonionic polyether, a nonionic natural polymer, a nonionic modified natural polymer, and a block polymer or a graft copolymer having two or more kinds of these polymers as constitutional units.

Among these nonionic hydrophilic polymers, a nonionic polyether, a nonionic polyester, a nonionic polyamino acid, or a nonionic synthetic polypeptide is preferable, a nonionic polyether or a nonionic polyester is more preferable, a nonionic polyether or a nonionic monoalkoxy polyether is even more preferable, and polyethylene glycol (hereinafter, polyethylene glycol will be also called PEG) is particularly preferable. That is, preferably, the lipid nanoparticles can contain PEG-bound lipids.

The lipid having a nonionic hydrophilic polymer is not particularly limited, and examples thereof include PEG-modified diacylphosphoethanolamine, a diacylglycerol PEG derivative, monoacylglycerol PEG derivative, a dialkylglycerol PEG derivative, a cholesterol PEG derivative, a ceramide PEG derivative, and the like. Among these, a PEG-modified diacylphosphoethanolamine and a diacylglycerol PEG is preferable. The acyl group in the PEG-modified diacylphosphoethanolamine and the diacylglycerol PEG preferably has 14 or more carbon atoms, more preferably 16 or more carbon atoms.

The weight average molecular weight of the nonionic hydrophilic polymer is preferably 100 to 10000, more preferably 500 to 5000, and even more preferably 750 to 3000.

The nonionic hydrophilic polymer chain may be branched or may have a substituent such as a hydroxymethyl group.

Preferred examples of the lipid having a nonionic hydrophilic polymer include the following lipids.
DMG-mPEG2000: 1,2-dimiristyl-rac-glycero-3-methoxypolyethylene glycol-2000
DPG-mPEG2000: 1,2-dipalmitoyl-rac-glycero-3-methoxypolyethylene glycol-2000
DSG-mPEG2000: 1,2-distearoyl-rac-glycero-3-methoxypolyethylene glycol-2000

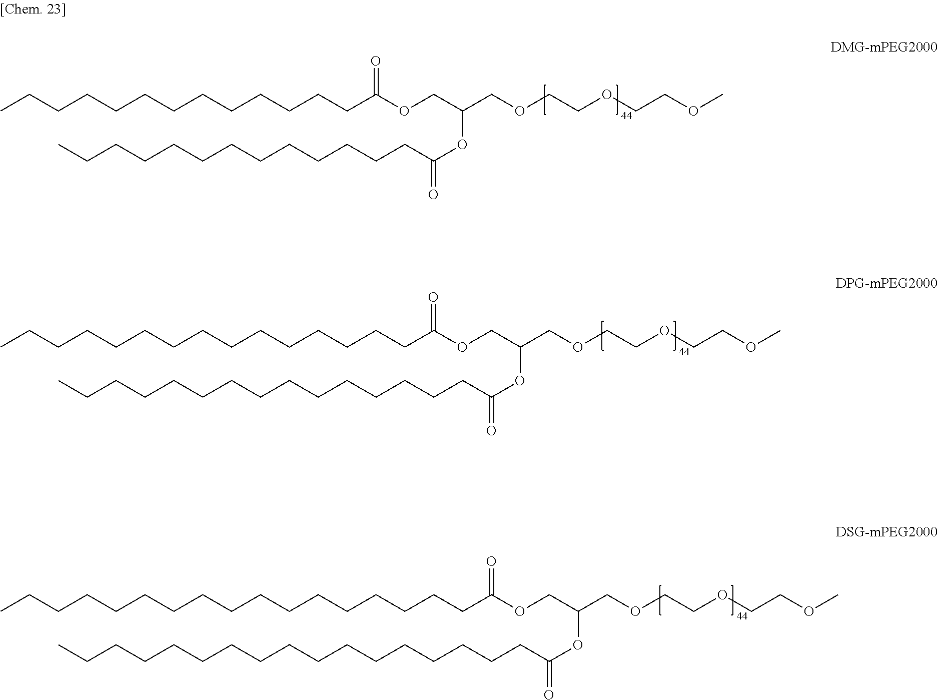

When a targeting molecule is bound to a lipid having a nonionic hydrophilic polymer, a lipid having a reactive group (for example, a maleimide group, thiol group, orthopyridyl disulfide (OPSS) group, N-hydroxysuccinimide (NHS) group, alkyne group, dibenzocyclooctine (DBCO) group, azide group, amino group, carboxyl group etc.) for binding the targeting molecule may be used as all or part of a lipid having nonionic hydrophilic polymer.

In the lipid composition of the present invention, the amount of the lipid having a nonionic hydrophilic polymer with respect to the total amount of lipids is preferably 0.1 mol % to 10 mol %, more preferably 0.3 mol % to 8 mol %, further preferably 0.5 mol % to 5 mol % and even more preferably 1 mol % to 3 mol %.

### Therapeutic Agent

The lipid composition of the present invention contains a therapeutic agent. As the therapeutic agent, nucleic acids such as polynucleotides are preferable. The nucleic acid such as a polynucleotide may be either DNA or RNA, and may be plasmid, single-stranded DNA, double-stranded DNA, siRNA (small interfering RNA), miRNA (micro RNA), mRNA, single guide RNA (sgRNA), antisense oligonucleotide (also known as ASO), ribozyme, aptamer, decoy nucleic acid, guide RNA (gRNA) used in genome editing and the like. It may also contain modified nucleic acids. When sgRNA and mRNA are used, sgRNA and mRNA may be contained separately or together in the lipid composition. Preferably, sgRNA and mRNA may be contained together in the lipid composition.

In the lipid composition of the present invention, the weight ratio of the lipid to the therapeutic agent is preferably 5 to 100, more preferably 5 to 70, still more preferably 5 to 40, and particularly preferably 5 to 35.

### Method for Manufacturing Composition

The method for manufacturing the lipid composition of the present invention will be described.

The method for manufacturing the lipid composition is not limited. For example, the lipid composition can be manufactured by a method in which all of the constituent components of the lipid particles or some of oil-soluble components of the lipid particles are dissolved in an organic solvent or the like such that an oil phase is formed, water-soluble components of the lipid particles are dissolved in water such that a water phase is formed, and the oil phase and the water phase are mixed together. A micromixer may be used for mixing, or an emulsifying machine such as a homogenizer, an ultrasonic emulsifying machine, or a high-pressure injection emulsifying machine may be used for emulsification.

Alternatively, the lipid composition can also be manufactured by a method in which a lipid-containing solution is subjected to evaporation to dryness using an evaporator under reduced pressure or subjected to spray drying using a spray drier such that a dried mixture containing a lipid is prepared, and the mixture is added to an aqueous solvent and further emulsified using the aforementioned emulsifying machine or the like.

One of the examples of the method for manufacturing the lipid particles containing a nucleic acid is a method including
a step (a) of dissolving the constituent components of the lipid particles containing the compound according to an embodiment of the present invention in an organic solvent so as to obtain an oil phase;
a step (b) of mixing the oil phase obtained in the step (a) with a water phase containing a nucleic acid;
a step (c) of diluting the mixed solution containing the oil phase and the water phase obtained in step (b) so as to obtain a dispersion liquid of nucleic acid-containing lipid composition; and
a step (d) of removing the organic solvent from the dispersion liquid of the nucleic acid lipid composition obtained in the step (c).

In the step (a), the lipid components are dissolved in an organic solvent (an alcohol such as ethanol, an ester, or the like). The total lipid concentration is not particularly limited, but is generally 1 mmol/L to 100 mmol/L, preferably 3 mmol/L to 50 mmol/L, and more preferably 5 mmol/L to 30 mmol/L.

In the step (b), the water phase can be obtained by dissolving a nucleic acid (for example, siRNA, an antisense nucleic acid, mRNA or the like) in water or a buffer. If necessary, a component such as an antioxidant can be added. The mixing ratio (volume ratio) of water phase:oil phase is preferably 5:1 to 1:1 and more preferably 4:1 to 2:1.

In the step (b), the mixed solution can be diluted with water or a buffer (for example, phosphate buffered saline (PBS) or the like).

In the step (c), as the method of removing the organic solvent from the dispersion liquid of the lipid composition, a general method can be used without particular limitation. For example, by dialyzing the dispersion liquid with the phosphate buffered saline, the organic solvent can be removed.

If necessary, the lipid composition can be subjected to sizing. Although the sizing method is not particularly limited, an extruder or the like can be used to reduce the particle size.

### Composition

The composition of the present invention may be lipid particle. The lipid particle means a particle composed of a lipid, and includes a composition having any structure selected from a lipid aggregate (for example, lipid nanoparticles) in which the lipid is aggregated, a micelle, and a liposome. However, the structure of the lipid particles is not limited to these as long as the composition contains lipids.

The form of the lipid particles can be checked by electron microscopy, structural analysis using X-rays, and the like. For example, by a method using Cryo transmission electron microscopy (CryoTEM method), it is possible to check, for example, whether a lipid particle such as a liposome has a structure composed of a bimolecular lipid membrane structure (lamella structure) and an inner water layer or a structure composed of an inner core with a high electron density and packed with constituent components including a lipid. The X-ray small angle scattering (SAXS) analysis also makes it possible to check whether or not a lipid particle has a bimolecular lipid membrane structure (lamella structure).

When the lipid composition of the present invention is a particle, the particle size is not particularly limited, but is preferably 10 to 1,000 nm, more preferably 30 to 500 nm, and even more preferably 50 to 250 nm. The particle size of the lipid particles can be measured by a general method (for example, a dynamic light scattering method, a laser diffraction method, or the like).

When the lipid composition of the present invention is a particle, the zeta potential of the particle is not particularly limited, but is preferably −20 to +20 mV, and more preferably −10 to 10 mV. The zeta potential in the present invention is a value measured by the *** method obtained by diluting the lipid composition in a phosphate buffer solution, but the method is not limited thereto.

The pKa of the lipid composition of the present invention is not particularly limited, but is preferably 9 to 4, more preferably 8 to 5, and even more preferably 7.5 to 6. The pKa of the lipid composition in the present invention adopts the value measured by the TNS assay, but is not limited to this.

### Use of Lipid Composition

As an example of the use of the lipid composition in the present invention, a therapeutic agent (for example, nucleic acid) can be introduced into the cell by introducing the lipid composition containing nucleic acid into the cell. That is, the lipid composition of the present invention can be used as a composition for introducing nucleic acid into cells.

Further, the lipid composition of the present invention can be used as a pharmaceutical composition for nucleic acid delivery in vivo. In the present invention, in particular, the therapeutic agent can be delivered to the hematopoietic stem/progenitor cell or mesenchymal stem cells. Therefore, examples of organs to which the therapeutic agent can be delivered include bone marrow, spleen and the like.

Further, when the lipid composition of the present invention contains a nucleic acid having a medicinal use, the lipid composition can be administered to a living body as a nucleic acid medicine. When the lipid composition of the present invention is used as a nucleic acid drug, the lipid composition of the present invention alone may be administered to a living body, or the lipid composition may be mixed with a pharmaceutically acceptable carrier (eg, an administration medium such as saline or phosphate buffer) and administered to a living body. That is, the lipid composition of the present invention may further contain a pharmaceutically acceptable carrier.

The concentration of the lipid composition in the mixture with the pharmaceutically acceptable carrier is not particularly limited and can generally be 0.05% by weight to 90% by weight. Further, other pharmaceutically acceptable additives such as a pH adjustment buffer and an osmotic pressure adjustment agent may be added to the nucleic acid drug containing the lipid composition of the present invention.

The route of administration for administering the lipid composition of the present invention is not particularly limited. The lipid composition can be administered by any method. Examples of the administration method include oral administration and parenteral administration (intra-articular administration, intravenous administration, intra-arterial administration, subcutaneous administration, intracutaneous administration, intravitreal administration, intraperitoneal administration, intramuscular administration, intravaginal administration, intravesical administration, intrathecal administration, pulmonary administration, rectal administration, colonic administration, buccal administration, nasal administration, intracisternal administration, inhalation, and the like). Among these, parenteral administration is preferable. As the method of administration, intravenous injection, subcutaneous injection, intracutaneous injection, or intramuscular injection is preferable. Intravenous injection or intramuscular injection is particularly preferable. As the administration, nucleic acid delivery can also be performed by local administration in vivo. The lipid composition of the present invention can also be administered by direct injection into the diseased site.

The dosage form of the lipid particles according to the embodiment of the present invention is not particularly limited. For oral administration, the lipid composition of the present invention can be used in the form of tablets, troches, capsules, pills, suspension, syrup, and the like by being combined with an appropriate excipient. In addition, additives such as an antioxidant, a buffer, a bacteriostat, an isotonic sterile injection, a suspending agent, a solubilizer, a thickener, a stabilizer, and a preservative can be appropriately incorporated into formulations suitable for parenteral administration.

### Use of Lipid Nanoparticle as Nucleic Acid Delivery Carrier

The lipid particles in the present invention can retain a nucleic acid at a high encapsulation rate. Therefore, the lipid particles are extremely useful as a nucleic acid delivery carrier. According to the nucleic acid delivery carrier using the present invention, for example, by mixing the obtained lipid particles with a nucleic acid or the like and performing transfection in vitro or in vivo, the nucleic acid and the like can be introduced into cells. Furthermore, the nucleic acid delivery carrier using the present invention is also useful as a nucleic acid delivery carrier in nucleic acid drugs. That is, the lipid particles according to the embodiment of the present invention are useful as a composition for in vitro or in vivo (preferably in vivo) delivery of a nucleic acid.

Next, the present invention will be described based on examples, but the present invention is not limited thereto.

### Drugs With Immune Suppressive Effect or CD117 Kinase Activity Inhibitor

Antihistamines (preferred)
   Examples of H1 histamine blocker
      Acrivastine, Alimemazine, Amitriptyline, Amoxapine, Aripiprazole, Azelastine, Bilastine, Bromodiphenhydramine, Brompheniramine, Buclizine, Carbinoxamine, Cetirizine, Chlophedianol, Chlorodiphenhydramine, Chlorpheniramine, Chlorpromazine, Chlorprothixene, Chloropyramine, Cinnarizine, Clemastine, Clomipramine, Clozapine, Cyclizine, Cyproheptadine, Desloratadine, Dexbrompheniramine, Dexchlorpheniramine Dimenhydrinate, Dimetindene, Diphenhydramine, Dosulepin, Doxepin, Doxylamine, Ebastine, Embramine, Fexofenadine, Fluoxetine, Hydroxyzine, Imipramine, Ketotifen, Levocabastine, Levocetirizine, Levomepromazine, Loratadine, Maprotiline, Meclizine, Mianserin, Mirtazapine, Olanzapine, Olopatadine, Orphenadrine, Periciazine, Phenindamine, Pheniramine, Phenyltoloxamine, Promethazine, Pyrilamine, Quetiapine, Rupatadine, Setastine, Setiptiline, Trazodone, Tripelennamine Triprolidines
      Cetirizine and Diphenhydramine are most preferred.
   H2 histamine blocker
      Cimetidine Famotidine Lafutidine Nizatidine Ranitidine Roxatidine Tiotidine
   Mast cell stabilizers
      cromolyn sodium, Nedocromil
Corticosteroids (preferred)
   prednisone (Deltasone, Orasone)
   prednisolone (Millipred)
   methylprednisolone (Medrol, Depo-Medrol, Solu-Medrol)
   bethamethasone, (Celestone)
   dexamethasone (Dexamethasone Intensol)
   budesonide (Entocort EC)
   triamcinolone acetonide (Aristospan Intra-Articular, Aristospan Intralesional, Kenalog)
   Examples of steroids
   Progesterone-type steroids:
   Flugestone, Fluorometholone, Medrysone (hydroxymethylprogesterone), Prebediolone acetate, chlormadinone acetate, cyproterone acetate, medrogestone, medroxyprogesterone acetate, megestrol acetate, and segesterone acetate
   Hydrocortisone-type steroids:
   Chloroprednisone, Cloprednol, Difluprednate, Fludrocortisone, Fluocinolone, Fluperolone, Fluprednisolone, Loteprednol, Methylprednisolone, Prednicarbate, Prednisone, Prednisolone, Tixocortol, Triamcinolone,
   Methasone-type steroids:
   Alclometasone, Bethamethasone, Beclometasone, Clobetasol, Clobetasone, Clocortolone, Desoximetasone, Dexamethasone, Diflorasone, Difluocortolone, Fluclorolone, Flumetasone, Fluocortin, Fluocortolone, Fluprednidene, Fluticasone, Fluticasone furoate, Halometasone, Meprednisone, Mometasone,
   Mometasone furoate, Paramethasone, Prednylidene, Rimexolone, Ulobetasol (halobetasol),
   Acetonides and related stedoids:
   Amcinonide, Budesonide, Ciclesonide, Deflazacort, Desonide, Formocortal (fluoroformylone), Fluclorolone acetonide (flucloronide), Fludroxycortide (flurandrenolone, flurandrenolide), Flunisolide, Fluocinolone acetonide, Fluocinonide, Halcinonide, Triamcinolone acetonide,
   Other steroids:
   Cortivazol, RU-28362 (6-methyl-11β,17β-dihydroxy-17α-(1-propynyl)androsta-1,4,6-trien-3-one)
CD117 kinase activity inhibitor (preferred)
   Axitinib, Cabozatinib, Dasatinib, Flumatinib, Imatinib, Imetelstat, Masatinib, Midostaurin, Nilotinib, Pazopanib, Sorafenib, Sunitinib, Toceranib,
Acetaminophen
Nonsteroidal anti-inflammatory drug: NSAIDS
   Salicylates such as Aspirin, Diflunisal, Salicylic acid and its salts, Salsalate,
   Propionic acid derivatives such as Ibuprofen, Dexibuprofen, Naproxen, Fenoprofen, Ketoprofen, Dexketoprofen, Flurbiprofen, Oxaprozin, Loxoprofen, Pelubiprofen, Zaltoprofen, Fenbufen, Tiaprofenic acid, Carprofen
   Acetic acid derivatives such as Indomethacin, Acemetacin, Tolmetin, Sulindac, Etodolac, Ketorolac, Diclofenac, Fenclofenac, Aceclofenac, Bromfenac, Fentiazac, Nabumetone,
   Enolic acid (oxicam) derivatives such as Piroxicam, Meloxicam, Tenoxicam, Droxicam, Lornoxicam, Isoxicam, Phenylbutazone,
   Anthranilic acid derivatives such as Mefenamic acid, Meclofenamic acid, Flufenamic acid, Tolfenamic acid, Etofenamate,
   Selective COX-2 inhibitors such as Celecoxib, Rofecoxib, Valdecoxib, Parecoxib, Lumiracoxib, Etoricoxib, Firocoxib
   Sulfonanilides such as Nimesulide

### Other Possible Pretreatment

Janus kinase inhibitors
   tofacitinib (Xeljanz)
Calcineurin inhibitors
   cyclosporine (Neoral, Sandimmune, SangCya)
   tacrolimus (Astagraf XL, Envarsus XR, Prograf)
mTOR inhibitors
   sirolimus (Rapamune)
   everolimus (Afinitor, Zortress)
IMDH inhibitors
   azathioprine (Azasan, Imuran)
   leflunomide (Arava)
   mycophenolate (CellCept, Myfortic)
Biologics
   abatacept (Orencia)
   adalimumab (Humira)
   anakinra (Kineret)
   certolizumab (Cimzia)
   etanercept (Enbrel)
   golimumab (Simponi)
   infliximab (Remicade)
   ixekizumab (Taltz)
   natalizumab (Tysabri)
   rituximab (Rituxan)
   secukinumab (Cosentyx)
   tocilizumab (Actemra)
   ustekinumab (Stelara)
   vedolizumab (Entyvio)
   basiliximab (Simulect)
   daclizumab (Zinbryta)

### EXAMPLES

### Materials and Methods

### siRNA

The following custom siRNA was manufactured by Horizon.

### siCD45 siRNA Sequence

Sense strand: mCmUGGmCmUGAAmUmUmUmCAGAGmCAdTdT,
Antisense strand: UGCUCUGAAAUUmCAGCmCAGdTdT
   Abbreviations of nucleotide monomers used in nucleic acid sequence representation. It will be understood that these monomers, when present in an oligonucleotide, are mutually linked by 5′-3′-phosphodiester bonds.

### Abbreviation Nucleotide(s)

A: Adenosine-3′-phosphate, C: cytidine-3′-phosphate, G: guanosine-3′-phosphate, U: Uridine-3′-phosphate,
mA: 2′-O-methyladenosine-3′-phosphate, mC: 2′-O-methylcytidine-3′-phosphate, mG: 2′-O-methylguanosine-3′-phosphate, mU: 2′-O-methyluridine-3′-phosphate,
dT: 2′-deoxythymidine-3′-phosphate, dTs: 2′-deoxythymidine-3′-phosphorothioate

### mRNA

Cre mRNA can be purchased from TriLink.

### Components of Lipid Composition

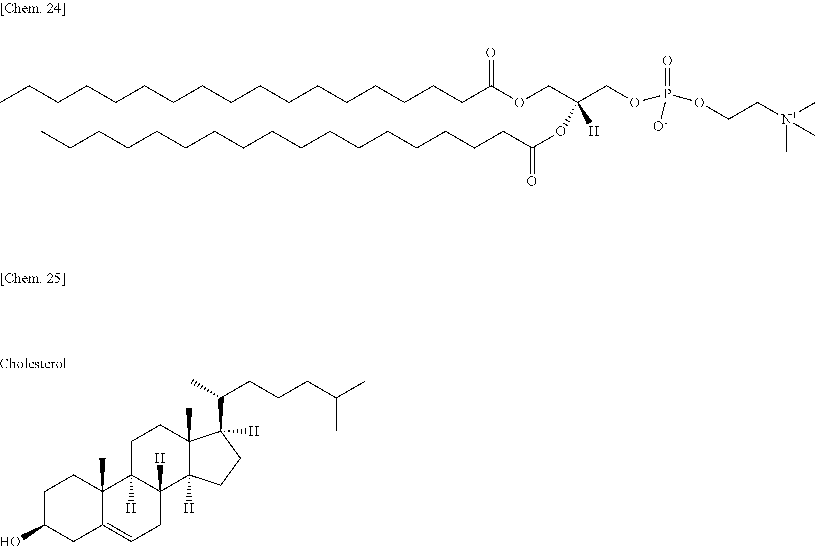

DMG-mPEG2000: 1,2-Dimiristyl-rac-Glycero-3-methoxypolyethylene glycol-2000
DPG-mPEG2000: 1,2-dipalmitoyl-rac-glycero-3-methoxypolyethylene glycol-2000
DSG-mPEG2000: 1,2-Distearoyl-rac-Glycero-3-methoxypolyethylene glycol-2000

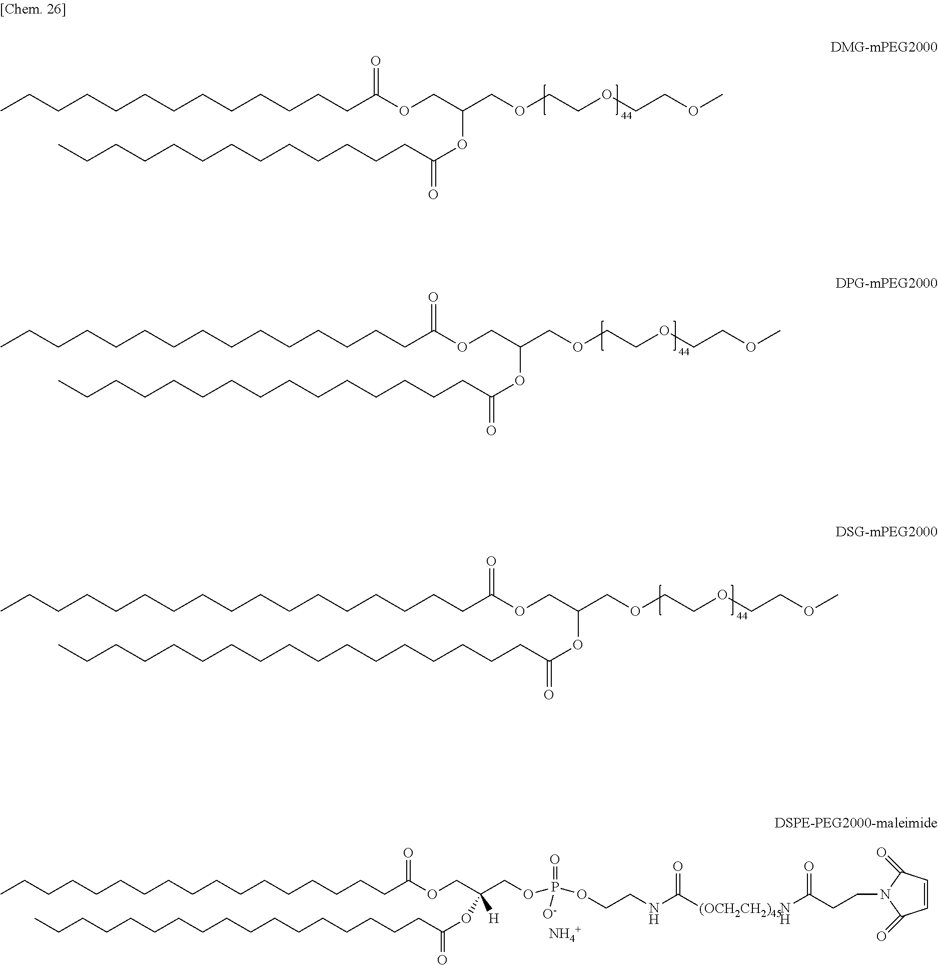

### Chemical Structure of Ionizable Lipids

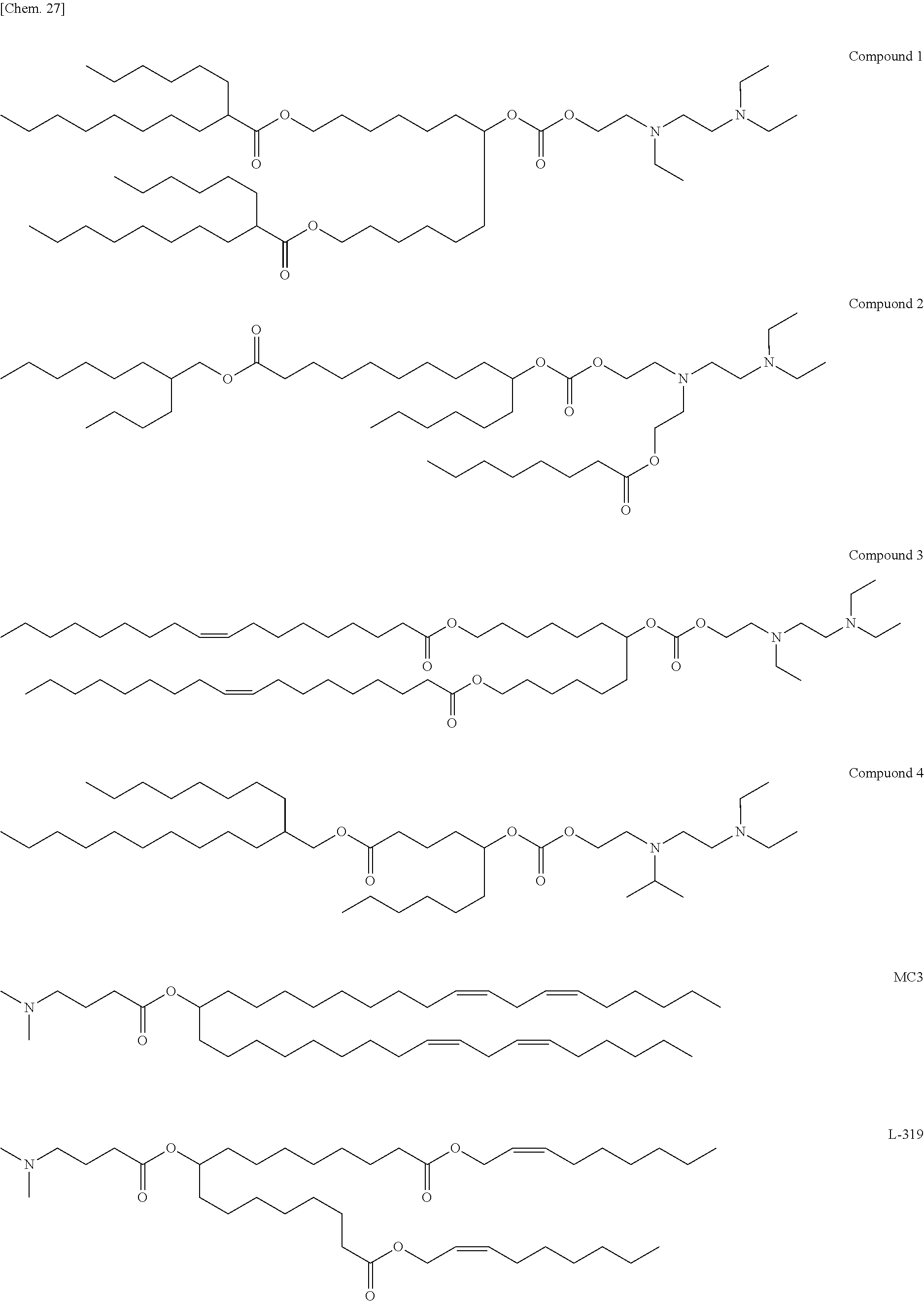

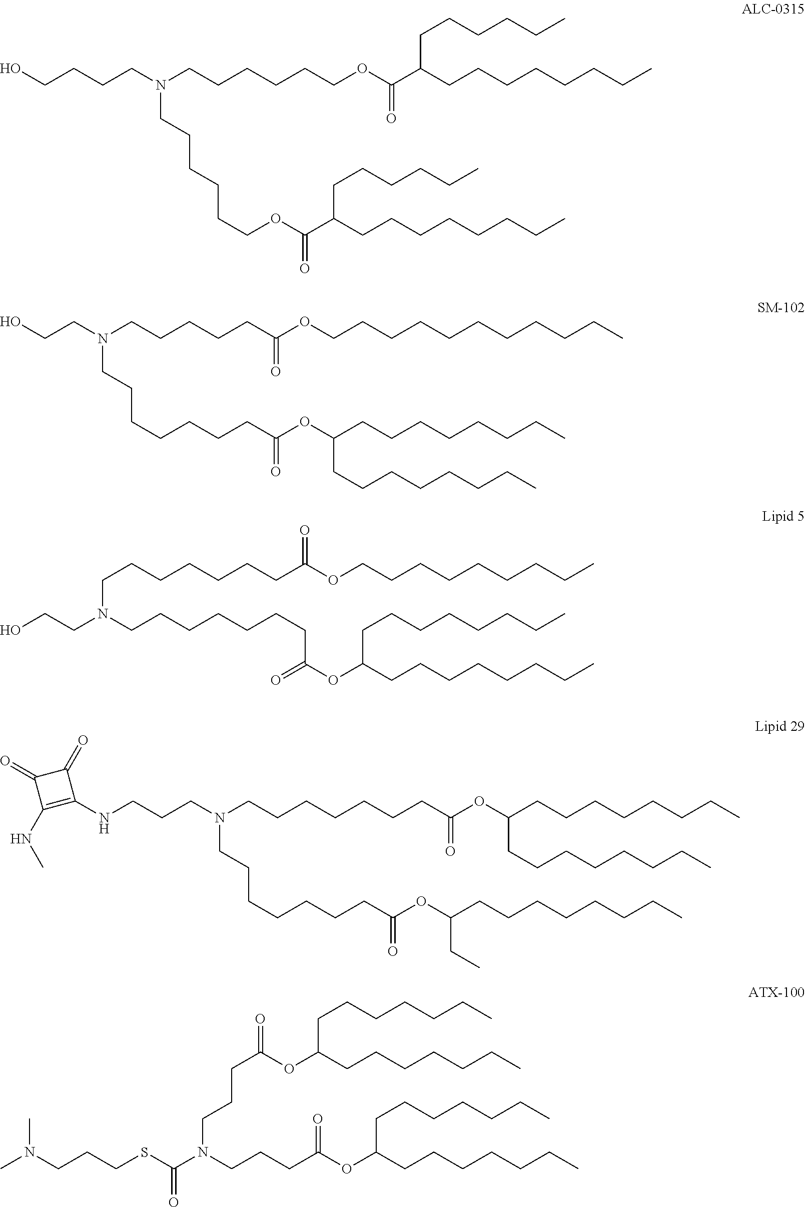

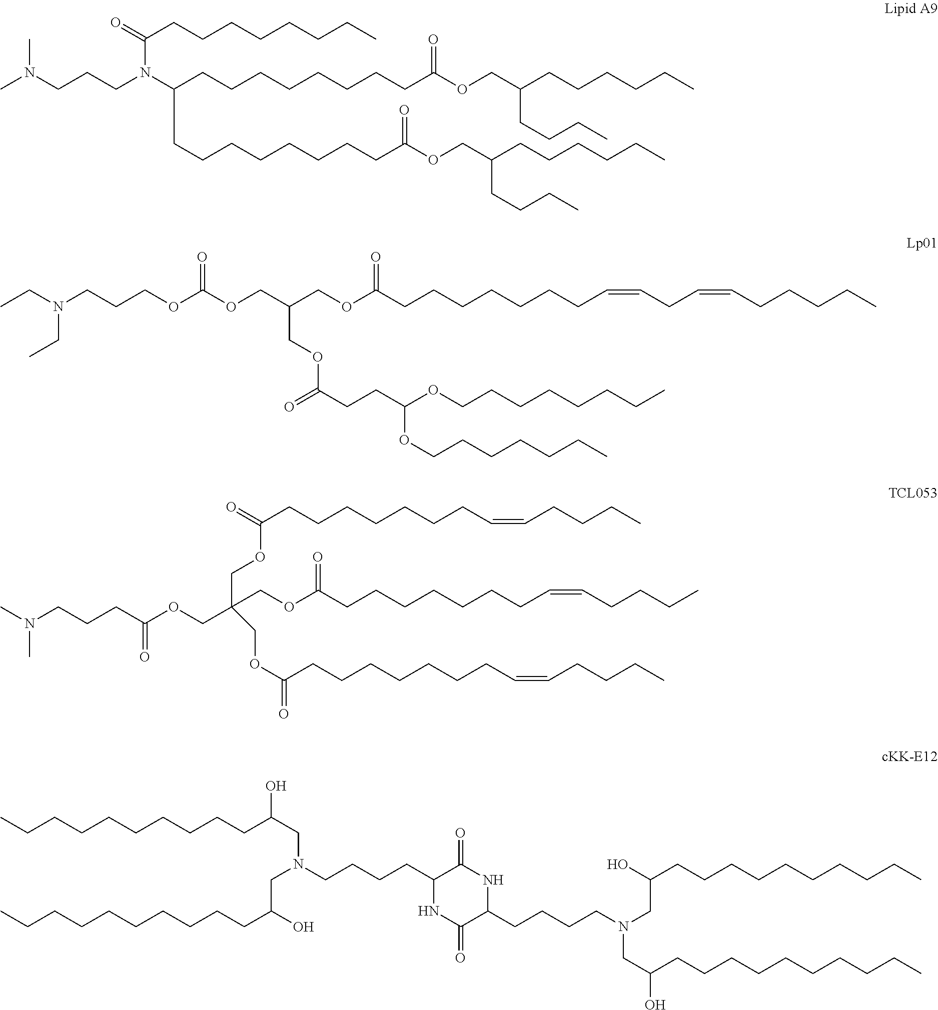

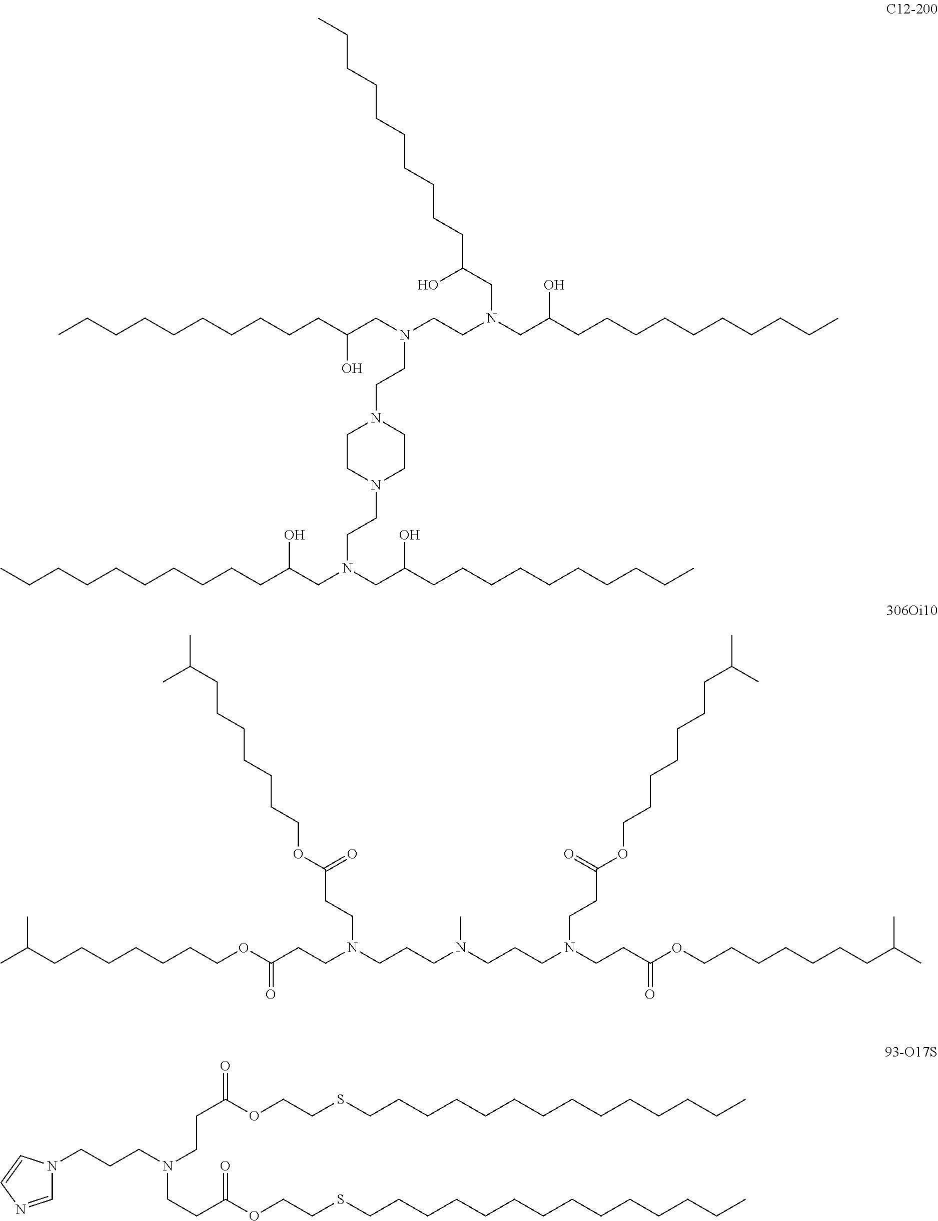

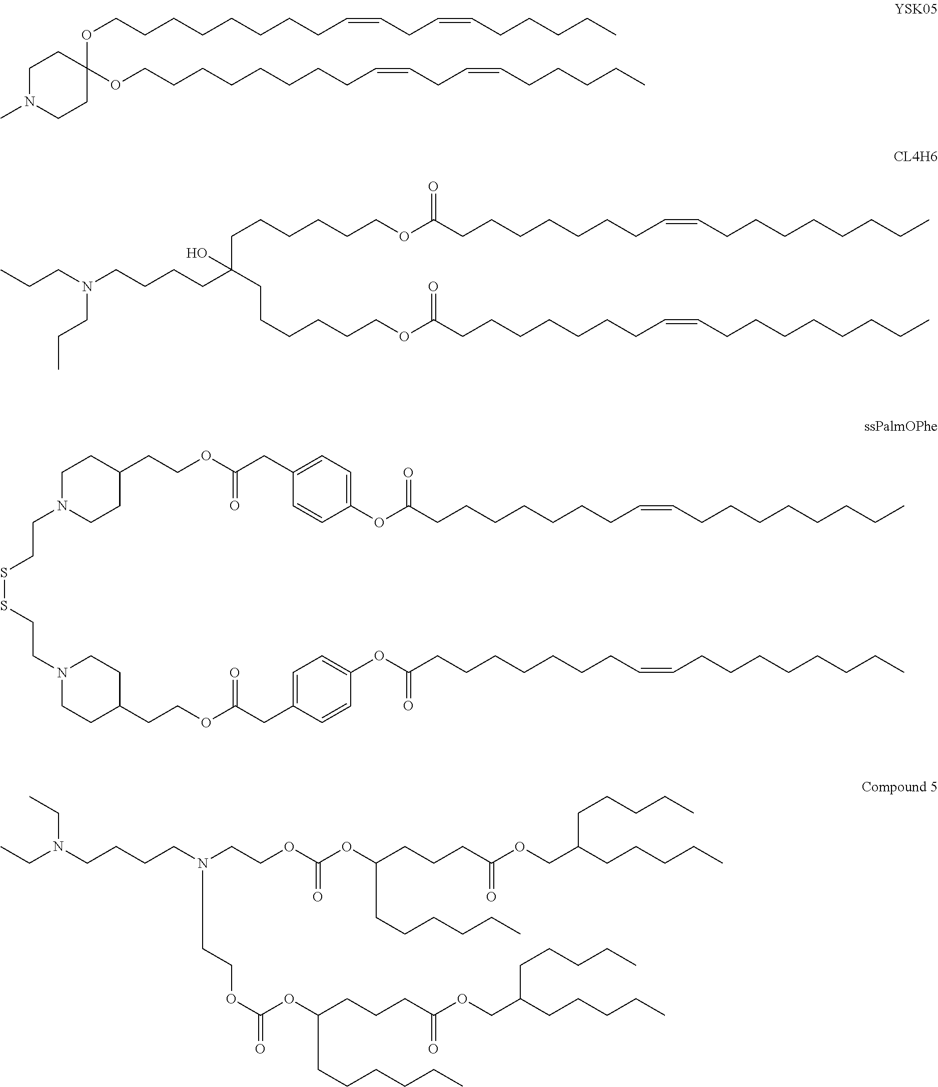

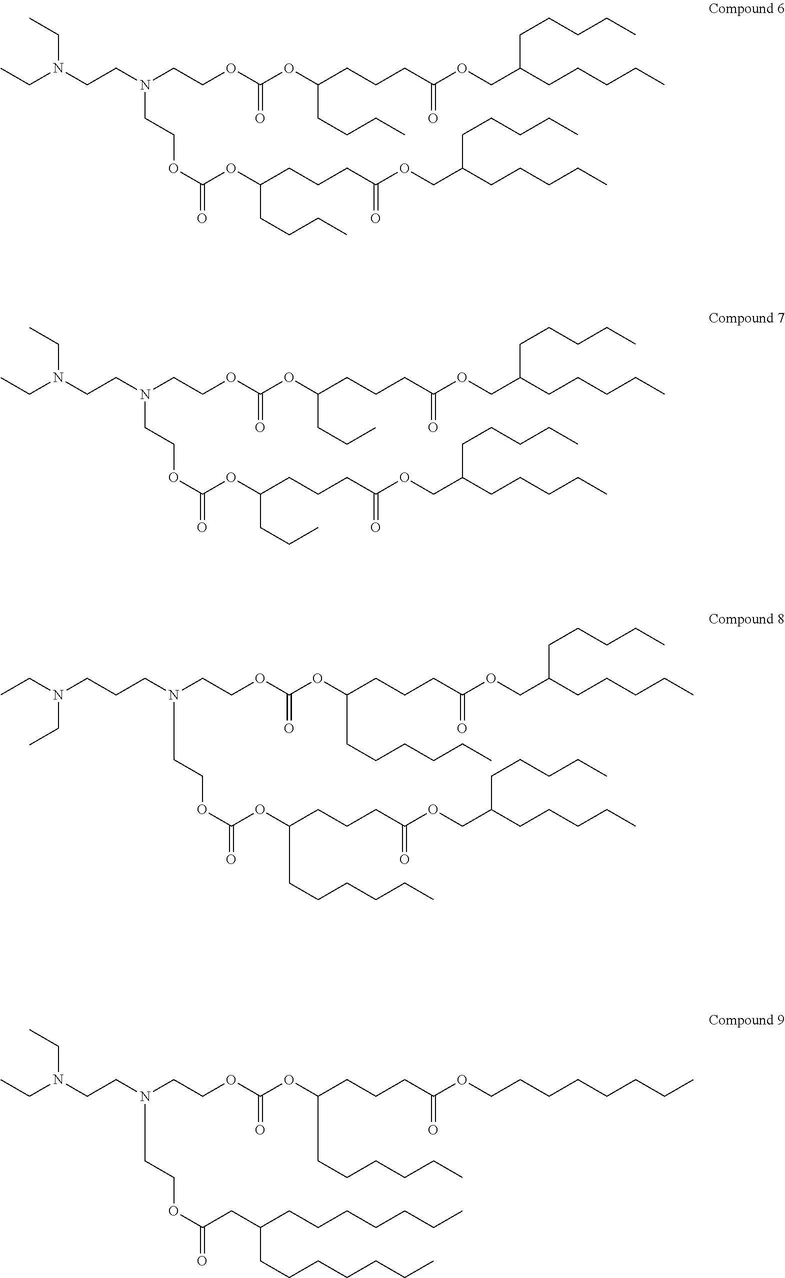

### Lipid Nanoparticle Formulation

siRNA was diluted in 10 mM citrate buffer, pH 3.0, (aqueous phase) while the appropriate amounts of lipids were co-dissolved in 200 proof ethanol (ethanol phase). Nanoparticles formulated via microfluidic device were synthesized at a 3:1 v/v ratio of the aqueous phase to the ethanol phase. Lipid nanoparticles were then dialyzed against PBS with 10 mM EDTA in a 20 kDa MWCO cassette at 4° C. or room temperature overnight.

### Antibody-Conjugation to Lipid Nanoparticles

Anti-CD117 antibody (Clone 2B8, Bio X Cell) was reduced with 5 eq. of TCEP (10 mM in PBS). Similarly, rat IgG2b isotype control (anti-keyhole limpet hemocyanin, Bio X Cell) was reduced with 5 eq. of 10 mM TCEP. The antibody was reduced by incubating at 37 C for one hour with gentle shaking. After incubation, excess TCEP was removed by Zeba 7k MWCO desalting column.

Maleimide-lipid nanoparticle dispersion was mixed with reduced antibody solution at a molar concentration ranging from 1:100 to 1:5 reduced antibody to maleimide and placed on end-over-end mixer at room temperature for 1 to 2 hours to allow for conjugation of the free thiols to the maleimides on the LNP. Thereafter, the mixture was stored at 4° C. until the purification step.

### Gel Filtration Purification of Antibody-Conjugated Lipid Nanoparticles

A reaction mixture containing antibody conjugated lipid nanoparticles was loaded on a gel filtration qEV column and fractionated with PBS as a mobile phase. The protein concentration of each fraction was measured to identify the fraction containing the antibody-lipid nanoparticle of interest. After collection, the antibody-lipid nanoparticle fractions were pooled and then concentrated with Amicon Ultra filters and the concentrated antibody-lipid nanoparticle was filtered through a 2 μm syringe filter and stored at 4° C.

### Particle Size Measurement

For lipid nanoparticles, particle size, PDI (polydispersity index), and ζ potential (i.e., zeta potential) were obtained using a Zetasizer (Malvern). For size measurement, LNPs were diluted in PBS at a 1/200 v/v ratio and z-average values were reported. For ζ potential measurement, LNPs were diluted in 0.1× PBS.

### Quantification of siRNA Concentration and Encapsulation

The siRNA concentration in dialyzed particles was determined via a modified Quant-iT RiboGreen RNA assay (Thermo Fisher). A nanoparticle dilution of ˜1 ng μL-1 siRNA was made in TE buffer (pH 8.5) and siRNA standards were made ranging from 2 ng μL-1 to 0.125 ng μL-1. 50 μL of each solution was added to separate wells in a 96-well black polystyrene plate. To each well was added either 50 μL of TE buffer or 50 μL of 2% Triton-X in TE. The plate was incubated at 37° C. for 15 minutes with shaking at 350 rpm. Following the incubation, the diluted RiboGreen reagent was added (100 μL per well), and the plate was incubated as before for 3 minutes. RiboGreen fluorescence was measured according to the supplied protocol using a Tecan plate reader, and the siRNA standard was used to determine nanoparticle siRNA concentration. It should be noted that two separate standards were made: one with and without Triton-X. The particles in TE buffer were used to determine un-encapsulated siRNA concentration and TE-TX, and encapsulation efficiency was determined via the following equation:

$$EE \% = ( 1 - \frac{{RNA}_{T E}}{{RNA}_{T X}} ) \times 100 \qquad [ Chem . 28 ]$$

### In Vitro Gene Silencing

EML cells were cultured in IMDM media supplemented with 20% HI-FBS, PenStrep, and 200 ng/ml murine stem cell factor (mSCF, Peprotech Inc.). Cells were plated in 96-well U-bottom plates with 50,000 cells/well in 100 μL volume of cell culture media. siRNA lipid nanoparticles were added to the cells at various concentrations and incubated for 40-48 hours. Three technical replicates of each transfection condition were used within each experiment. After incubation, cell culture media was removed and cells were washed with PBS.

After washing, 100 μL QuickExtract™ RNA Extraction Kit (Lucigen, Cat #QER090150) was used to extract total RNA from the EML cells. For qPCR, 2 μl of RNA extract was added to a master mix containing 0.5 μl B2M TaqMan Probe (Applied Biosystems Cat #Mm00437762_m1) or 0.5 μl CD45 TaqMan probe (Applied Biosystems cat #Mm01293577_m1) and 7 μl Luna™ Universal Probe One-Step RT-qPCR Kit (NEB Cat #E3006) per well in a 384 well plates. Real time PCR was done in a Light Cycler 480 (Roche). Each duplex was tested in two or three independent transfections, and each transfection was assayed in duplicate, unless otherwise noted.

To calculate relative fold change, real time data was analyzed using the ΔΔCt method and normalized to assays performed with cells transfected with the same concentration of siRNA against luciferase, or mock transfected cells. IC50s were calculated using a Graphpad Prism software.

### Bone Marrow Flow Cytometry

Mice were sacrificed and femur and tibia bones collected. Bone marrow cells were collected by cutting the femur and tibia in half followed by centrifugation of the bones in an Eppendorf tube at 13,000×g for 45 seconds. The resulting pellet was resuspended in 1 mL of PBS and filtered through a 70 μm strainer and washed with 10 mL PBS. After centrifugation, BM cells were lysed with 1 mL of RBC lysis buffer (Qiagen) for 10 minutes on ice with gentle shaking. After RBC lysis, bone marrow cells were transferred to a 96 well U-bottom plate for flow cytometry staining. Staining markers include lineage markers (CD3, Gr-1, CD11b, CD45R/B220, mTer-119), CD117, Sca1, and CD45.

Samples were analyzed on BD LSR Fortessa II and data was analyzed with FlowJo.

### <Example 1> In Vitro RNA Delivery to CD117+ Cells

The ability of anti-murine CD117 antibody modified lipid nanoparticles, as compared to lipid nanoparticle without antibody modification, to deliver mRNA into EML cells (ATCC CRL-11691), a murine CD117-positive stem cell factor-dependent lympho-hematopoietic progenitor cell line, was tested in vitro.

EML cells were cultured in IMDM media in the presence of 200 ng/ml murine stem cell factor (mSCF1, R&D Systems). Cells were plated in 96-well plates with 50,000 cells/well in 100 μL volume cell culture media containing various concentrations of siRNA-LNPs with three technical replicates within each experiment. After 40 h, cell culture media was removed and cells were washed with PBS.

QuickExtract™ RNA Extraction Kit (Lucigen, Cat #QER090150) was used to extract total RNA from the EML cells. 2 μl of RNA extract was added to a master mix containing 0.5 μl B2M TaqMan Probe (Applied Biosystems Cat #Mm00437762_m1) or 0.5 μl CD45 TaqMan probe (Applied Biosystems cat #Mm01293577_m1) and 7 μl Luna™ Universal Probe One-Step RT-qPCR Kit (NEB Cat #E3006) per well in a 384 well plates. Real time PCR was done in a Light Cycler 480 (Roche). Each duplex was tested in two or three independent transfections, and each transfection was assayed in duplicate, unless otherwise noted.

To calculate relative fold change, real time data was analyzed using the ΔΔCt method and normalized to assays performed with cells transfected with the same concentration of siRNA against luciferase, or mock transfected cells. IC50s were calculated using a Graphpad Prism software.

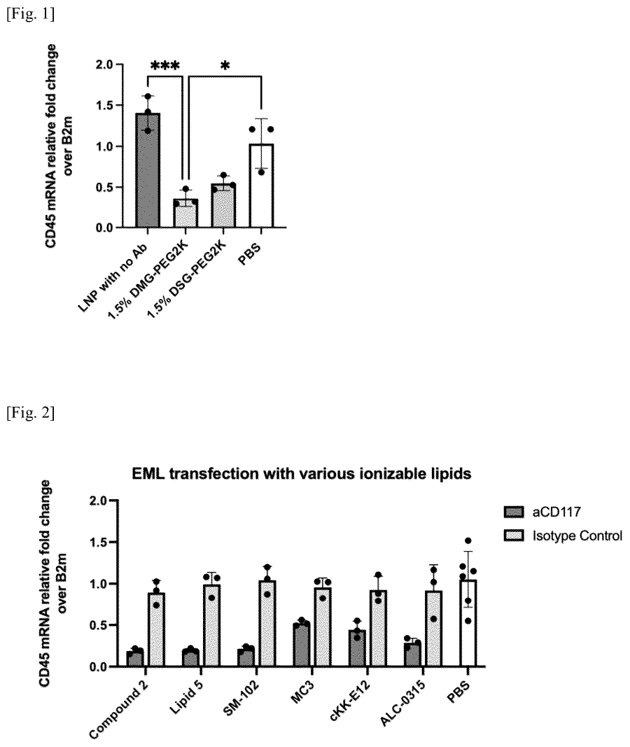

The result is shown in FIG. 1. FIG. 1 shows In vitro evaluation of CD45 gene silencing on EML cells using LNPs conjugated with aCD117.

TABLE 1

| | | | | PEG- | PEG- | Composition | |
|---|---|---|---|---|---|---|---|
| | Ionizable lipid | Phospholipid | Cholesterol | lipid 1 | lipid 2 | (mol %) | Antibody |
| LNP1 | COMPOUND 1 | DSPC | Cholesterol | DMG- | — | 50:10:38.5:1.5:0 | — |
| | | | | PEG2000 | | | |
| LNP2 | COMPOUND 1 | DSPC | Cholesterol | DMG- | DSPE- | 50:10:38:1.5:0.5 | Anti- |
| | | | | PEG2000 | PEG2000- | | murine |
| | | | | | maleimide | | CD117 |
| LNP3 | COMPOUND 1 | DSPC | Cholesterol | DSG- | DSPE- | 50:10:38:1.5:0.5 | Anti- |
| | | | | PEG2000 | PEG2000- | | murine |
| | | | | | maleimide | | CD117 |

### Example 2: In Vitro Generalization of Ionizable Lipids

To examine the generalizability of this anti-CD117 lipid nanoparticle in vitro, various ionizable lipids were formulated into LNPs containing siRNA against CD45 and then conjugated to either an antibody against murine CD117 or a rat IgG2b isotype control antibody. After conjugation, lipid nanoparticles were then added to EML cells at a dose of 50 ng siRNA per well. 40 hours after transfection, cells were washed with PBS and then resuspended and lysed in QuickExtract RNA solution. The knockdown of CD45 in treated cells was assayed using qPCR using the ΔΔCt method.

The result is shown in FIG. 2. FIG. 2 shows that knockdown of CD45 through aCD117-receptor interaction is generalizable to other ionizable lipids.

From the data, it was demonstrated that conjugation of anti-CD117 results in effective delivery with all of the tested ionizable lipids. It is understood that delivery is mediated through anti-CD117 antibody-receptor interaction given that conjugation of these lipid nanoparticles with an isotype control antibody showed no silencing.

TABLE 2

| | Ionizable | | | PEG- | PEG- | Composition | |
|---|---|---|---|---|---|---|---|
| LNP # | lipid | Phospholipid | Cholesterol | lipid 1 | lipid 2 | (mol %) | Antibody |
| LNP4 | COMPOUND | DSPC | Cholesterol | DMG- | DSPE- | 50:10:38:1.5:0.5 | Anti- |
| | 2 | | | PEG2000 | PEG2000- | | murine |
| | | | | | maleimide | | CD117 |
| LNP5 | COMPOUND | DSPC | Cholesterol | DMG- | DSPE- | 50:10:38:1.5:0.5 | Rat IgG |
| | 2 | | | PEG2000 | PEG2000- | | |
| | | | | | maleimide | | |
| LNP6 | Lipid 5 | DSPC | Cholesterol | DMG- | DSPE- | 50:10:38:1.5:0.5 | Anti- |
| | | | | PEG2000 | PEG2000- | | murine |
| | | | | | maleimide | | CD117 |
| LNP7 | Lipid 5 | DSPC | Cholesterol | DMG- | DSPE- | 50:10:38:1.5:0.5 | Rat IgG |
| | | | | PEG2000 | PEG2000- | | |
| | | | | | maleimide | | |
| LNP8 | SM-102 | DSPC | Cholesterol | DMG- | DSPE- | 50:10:38:1.5:0.5 | Anti- |
| | | | | PEG2000 | PEG2000- | | murine |
| | | | | | maleimide | | CD117 |
| LNP9 | SM-102 | DSPC | Cholesterol | DMG- | DSPE- | 50:10:38:1.5:0.5 | Rat IgG |
| | | | | PEG2000 | PEG2000- | | |
| | | | | | maleimide | | |
| LNP10 | MC3 | DSPC | Cholesterol | DMG- | DSPE- | 50:10:38:1.5:0.5 | Anti- |
| | | | | PEG2000 | PEG2000- | | murine |
| | | | | | maleimide | | CD117 |
| LNP11 | MC3 | DSPC | Cholesterol | DMG- | DSPE- | 50:10:38:1.5:0.5 | Rat IgG |
| | | | | PEG2000 | PEG2000- | | |
| | | | | | maleimide | | |
| LNP12 | cKK-E12 | DSPC | Cholesterol | DMG- | DSPE- | 50:10:38:1.5:0.5 | Anti- |
| | | | | PEG2000 | PEG2000- | | murine |
| | | | | | maleimide | | CD117 |
| LNP13 | cKK-E12 | DSPC | Cholesterol | DMG- | DSPE- | 50:10:38:1.5:0.5 | Rat IgG |
| | | | | PEG2000 | PEG2000- | | |
| | | | | | maleimide | | |
| LNP14 | ALC-0315 | DSPC | Cholesterol | DMG- | DSPE- | 50:10:38:1.5:0.5 | Anti- |
| | | | | PEG2000 | PEG2000- | | murine |
| | | | | | maleimide | | CD117 |
| LNP15 | ALC-0315 | DSPC | Cholesterol | DMG- | DSPE- | 50:10:38:1.5:0.5 | Rat IgG |
| | | | | PEG2000 | PEG2000- | | |
| | | | | | maleimide | | |

### Example 3: In Vivo RNA Delivery to Bone Marrow CD117+ Hematopoietic Stem and Progenitor Cells (DMG vs. DSG)

The ability of anti-CD117 antibody modified lipid nanoparticle to deliver RNA to HSPCs in vivo was evaluated in C57BL/6 mice. Lipid nanoparticles were formulated containing siCD45 and PEG-lipids of varying alkyl chain lengths. Different alkyl chain lengths DMG (C14) and DSG (C18) were included to examine the effect of LNP circulation time on delivery to bone marrow cells. After formulation, LNPs were conjugated to murine anti-CD117 and after conjugation unreacted antibody was removed with size exclusion chromatography.

Lipid nanoparticles were administered to mice through i.v tail vein injection at a dose of 1 mg/kg (20 ug) siRNA. After 72 hrs, mice were sacrificed and the bone (femur+tibia) was processed into single cell suspension and then stained for flow cytometry analysis. To analyze delivery to HSPCs, bone marrow cells were gated with standard mouse HSPC markers to identify cells that were negative for lineage markers and double positive for Sca1 and c-Kit (also known as LSK cells). Within this LSK population, the MFI of CD45 was analyzed to determine silencing.

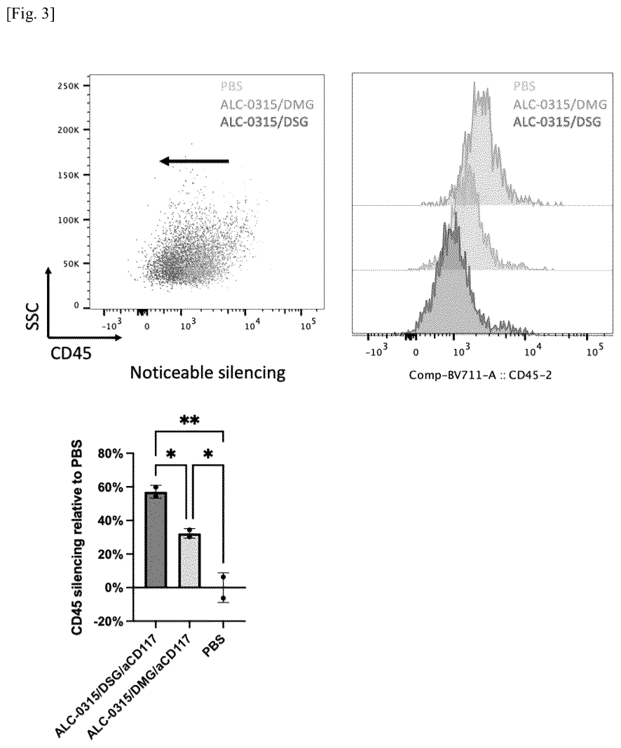

The result is shown in FIG. 3. FIG. 3 shows CD45 expression level in bone marrow LSK cells as quantified by flow cytometry. Both DMG and DSG formulations showed significant CD45 knockdown in bone marrow LSK cells. Use of DSG in the formulation showed a higher level of silencing (60%) compared to the silencing observed with DMG containing formulations (30%). Lipid nanoparticles modified with anti-CD117 antibody allows for effective RNA delivery to HSPCs in vivo. These results indicate that lipid nanoparticles with a longer circulation time are better for delivery.

TABLE 4
The formulations tested in mice

| | Ionizable | | | PEG- | PEG- | Composition | |
|---|---|---|---|---|---|---|---|
| LNP # | lipid | Phospholipid | Cholesterol | lipid 1 | lipid 2 | (mol %) | Antibody |
| LNP16 | ALC-0315 | DSPC | Cholesterol | DMG- | DSPE- | 50:10:38:1.5:0.5 | Anti- |
| | | | | PEG2000 | PEG2000- | | murine |
| | | | | | maleimide | | CD117 |
| LNP17 | ALC-0315 | DSPC | Cholesterol | DSG- | DSPE- | 50:10:38:1.5:0.5 | Anti- |
| | | | | PEG2000 | PEG2000- | | murine |
| | | | | | maleimide | | CD117 |

To further evaluate the delivery of mRNA to bone marrow CD117+ hematopoietic stem and progenitor cells, anti-CD117 conjugated LNPs were formulated with Cre recombinase mRNA and then administered into Ai14 reporter mice. These mice contain a loxP-flanked STOP cassette that prevents transcription of a fluorescent TdTomato protein. Upon Cre recombination, the cells will express TdTomato which allows for analysis of functional mRNA delivery at the cellular level with flow cytometry. Mice were injected via tail vein with anti-CD117 LNP containing Cre mRNA or PBS. Forty-eight hours after injection, mice were sacrificed and the bones (femur and tibia) were harvested and processed into a single cell suspension for flow cytometry analysis. TdTomato fluorescence was evaluated in HSPCs (defined as Lin−Sca1+c-Kit+) and long-term hematopoietic stem cells (or LT-HSCs, defined as Lin−Sca1+cKit+CD34−CD135−). LT-HSCs are capable of self-renewal and are the cells that are the most biologically significant when considering stem cell therapy.

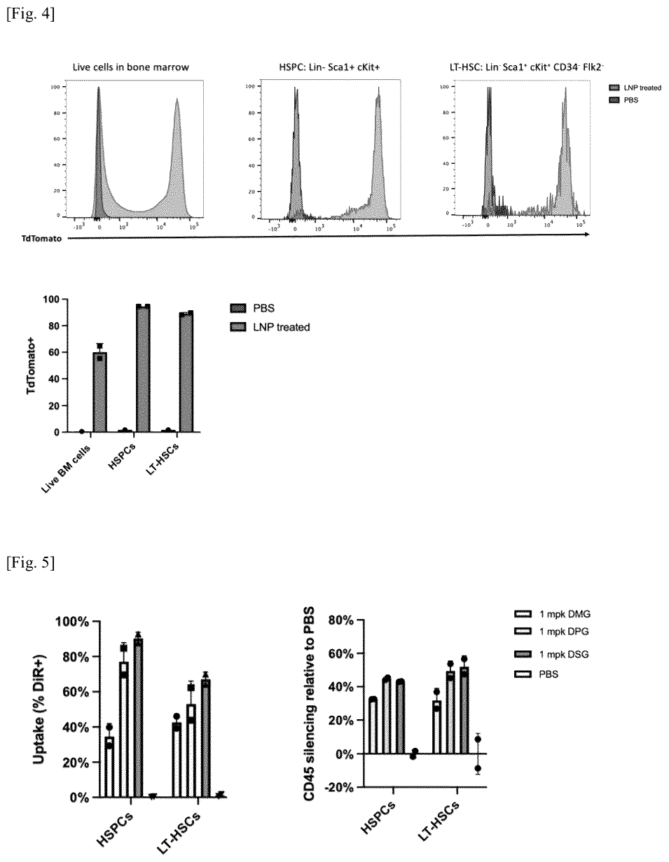

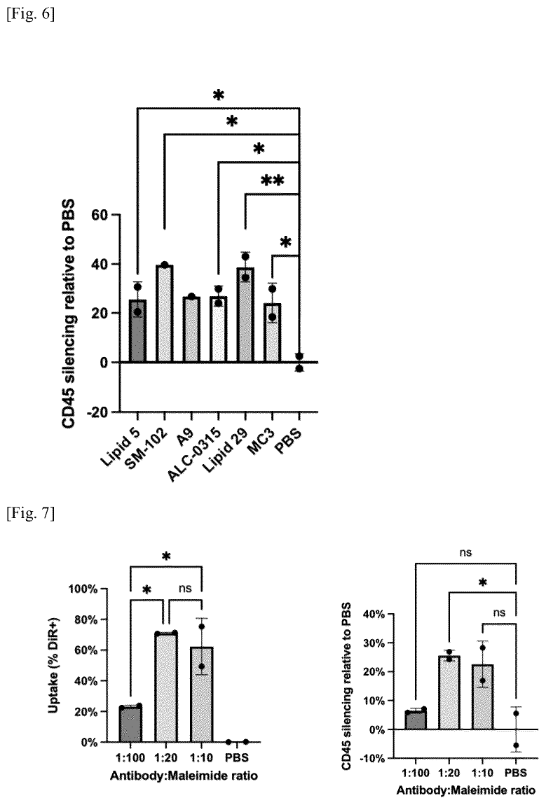

FIG. 4 shows in vivo Cre mRNA delivery in bone marrow HSPCs using Ai14 mouse model. LNP conjugated to aCD117 shows very high levels (>90%) of mRNA delivery to bone marrow HSPCs and LT-HSCs. As shown in FIG. 4, aCD117-LNP is able to achieve high levels of mRNA delivery and Cre recombination (around 90%) to both HSPCs and LT-HSCs. In addition, around 50% of all bone marrow cells expressed TdTomato.

### Example 4: In Vivo RNA Delivery to Bone Marrow CD117+ Hematopoietic Stem and Progenitor Cells 2 (DPG (C16) vs. DSG (C18))

To further evaluate the effect of PEG lipid alkyl chain length on the delivery efficacy of aCD117-LNPs, we compared DPG-PEG (C16) with DMG-PEG (C14) and DSG-PEG (C18) formulations. LNPs were formulated with siRNA against CD45 with the different PEG lipids. In addition, the LNPs were fluorescently labeled with the lipophilic dye DiR to track differences in uptake of these formulations. LNPs were administered to mice via tail vein at a dose of 1 mg/kg. 72 hours after administration, the bone marrow was collected and then processed into a single cell suspension. Uptake and silencing in bone marrow HSPCs was analyzed by flow cytometry.

The result is shown in FIG. 5. FIG. 5 shows (Left) LNP Uptake in HSPCs or LT-HSCs is correlated with longer alkyl chain length. DSG-PEG (C18) shows the highest level of uptake in the cell populations of interest. (Right) Level of functional gene silencing in HSPCs with different alkyl chain lengths.

TABLE 5
The formulations tested in mice

| | Ionizable | | | PEG- | PEG- | Composition | |
|---|---|---|---|---|---|---|---|
| LNP # | lipid | Phospholipid | Cholesterol | lipid 1 | lipid 2 | (mol %) | Antibody |
| LNP18 | ALC-0315 | DSPC | Cholesterol | DMG- | DSPE- | 50:10:38:1.5:0.5 | Anti- |
| | | | | PEG2000 | PEG2000- | | murine |
| | | | | | maleimide | | CD117 |
| LNP19 | ALC-0315 | DSPC | Cholesterol | DPG- | DSPE- | 50:10:38:1.5:0.5 | Anti- |
| | | | | PEG2000 | PEG2000- | | murine |
| | | | | | maleimide | | CD117 |
| LNP20 | ALC-0315 | DSPC | Cholesterol | DSG- | DSPE- | 50:10:38:1.5:0.5 | Anti- |
| | | | | PEG2000 | PEG2000- | | murine |
| | | | | | maleimide | | CD117 |

### Example 5: In Vivo Generalization of Ionizable Lipid

To examine the generalizability of this anti-CD117 lipid nanoparticle in vivo, various ionizable lipids were formulated into LNPs containing siRNA against CD45 and then conjugated to an antibody against murine CD117. LNPs were administered to mice via tail vein at a dose of 1 mg/kg. 72 hours after administration, the bone marrow was collected and then processed into a single cell suspension. CD45 silencing in bone marrow HSPCs was analyzed by flow cytometry. The formulations shown in Table 6 were prepared and tested.

The results are shown in FIG. 7. FIG. 7 shows functional knockdown of CD45 in HSPCs. LNPs were injected at a lower dose of 0.3 mg/kg RNA to evaluate the effects of ligand density on RNA delivery. Both uptake and silencing show an optimal ligand density for RNA delivery using anti-CD117 conjugated lipid nanoparticles. From the data, it was demonstrated that conjugation of anti-CD117 results in effective delivery with all of the tested ionizable lipids in vivo.

TABLE 6
The formulations tested in mice

| | Ionizable | | | PEG- | PEG- | Composition | |
|---|---|---|---|---|---|---|---|
| LNP # | lipid | Phospholipid | Cholesterol | lipid 1 | lipid 2 | (mol %) | Antibody |
| LNP21 | Lipid 5 | DSPC | Cholesterol | DSG- | DSPE- | 50:10:38:1.5:0.5 | Anti- |
| | | | | PEG2000 | PEG2000- | | murine |
| | | | | | maleimide | | CD117 |
| LNP22 | SM-102 | DSPC | Cholesterol | DSG- | DSPE- | 50:10:38:1.5:0.5 | Anti- |
| | | | | PEG2000 | PEG2000- | | murine |
| | | | | | maleimide | | CD117 |
| LNP23 | A9 | DSPC | Cholesterol | DSG- | DSPE- | 50:10:38:1.5:0.5 | Anti- |
| | | | | PEG2000 | PEG2000- | | murine |
| | | | | | maleimide | | CD117 |
| LNP24 | ALC- | DSPC | Cholesterol | DSG- | DSPE- | 50:10:38:1.5:0.5 | Anti- |
| | 0315 | | | PEG2000 | PEG2000- | | murine |
| | | | | | maleimide | | CD117 |
| LNP25 | Lipid 29 | DSPC | Cholesterol | DSG- | DSPE- | 50:10:38:1.5:0.5 | Anti- |
| | | | | PEG2000 | PEG2000- | | murine |
| | | | | | maleimide | | CD117 |
| LNP26 | MC3 | DSPC | Cholesterol | DSG- | DSPE- | 50:10:38:1.5:0.5 | Anti- |
| | | | | PEG2000 | PEG2000- | | murine |
| | | | | | maleimide | | CD117 |

### Example 6: In Vivo (or In Vitro) Antibody-Density Optimization

To optimize the antibody density on the surface of the LNPs, Ab-LNPs were prepared with various molar ratios of maleimide and antibody during conjugation (Table 7). Using more antibody during the conjugation reaction would result in more Abs on the surface of the LNP. In addition, Ab-LNPs were also labeled with a lipophilic dye DiR to track the uptake of these nanoparticles in vivo. These fluorescently labeled LNPs containing an siRNA against CD45 were intravenously injected to mice at 0.3 mg/kg. 72 hours after injection, the bone marrow was collected and both uptake and knockdown of CD45 in HSPCs (Lin− Sca1+ cKit+ cells) were evaluated with flow cytometry.

The results is shown in FIG. 7. FIG. 7 shows (Left) Uptake in HSPCs as determined by % of LSK cells that are DiR+. (Right) Functional knockdown of CD45 in HSPCs. LNPs were injected at a lower dose of 0.3 mg/kg RNA to evaluate the effects of ligand density on RNA delivery. Both uptake and silencing show an optimal ligand density for RNA delivery using anti-CD117 conjugated lipid nanoparticles.

TABLE 7
The formulations tested in mice

| | Ionizable | | | | |
|---|---|---|---|---|---|
| LNP # | lipid | Phospholipid | Cholesterol | PEG-lipid 1 | PEG-lipid 2 |
| LNP27 | ALC-0315 | DSPC | Cholesterol | DSG-PEG2000 | DSPE-PEG2000-maleimide |
| LNP28 | ALC-0315 | DSPC | Cholesterol | DSG-PEG2000 | DSPE-PEG2000-maleimide |
| LNP29 | ALC-0315 | DSPC | Cholesterol | DSG-PEG2000 | DSPE-PEG2000-maleimide |

| LNP # | Composition (mol %) | Antibody | Maleimide:Ab ratio |
|---|---|---|---|
| LNP27 | 50:10:38:1.5:0.5 | Anti-murine CD117 | 1:100 |
| LNP28 | 50:10:38:1.5:0.5 | Anti-murine CD117 | 1:20 |
| LNP29 | 50:10:38:1.5:0.5 | Anti-murine CD117 | 1:10 |

Using the optimum ligand density, the dose response in vivo of these Ab-LNPs were evaluated by administering doses of 0.1 mg/kh, 0.3 mg/kg, and 1 mg/kg to mice. 72 hours after LNP administration, mice were sacrificed, and the bone marrow was collected and processed into a single cell suspension. For the dose curve analysis, nanoparticle uptake and silencing were evaluated in various cell populations including HSPCs, LT-HSCs, mature immune cells, and CD117-cells.

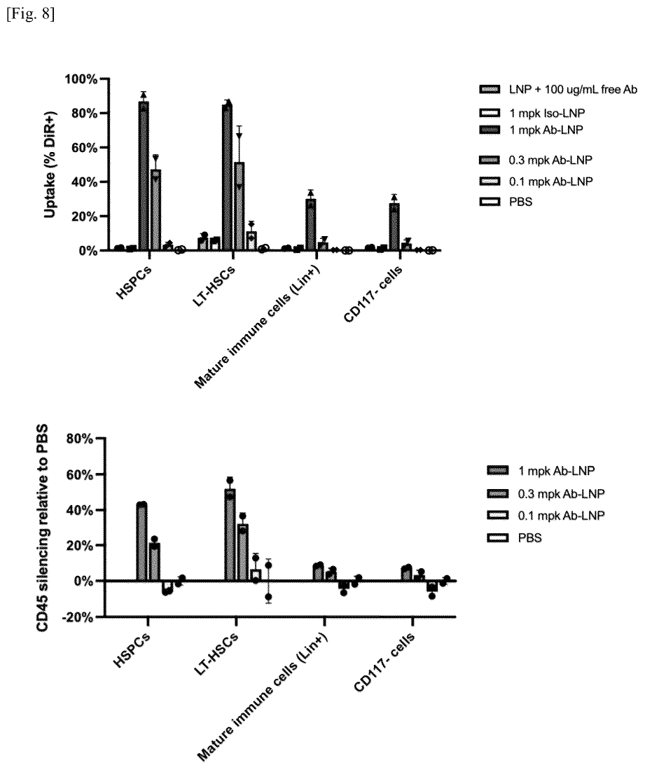

The result is shown in FIG. 8. FIG. 8 shows (Top) LNP uptake in various cell populations of the bone marrow as determined by % DiR positive. There is a dose dependent uptake in HSPCs and LT-HSCs. In addition, LNPs that were mixed with free Ab (unconjugated) as well as isotype control LNPs (Iso-LNP) do not show any uptake to HSPCs or LT-HSCs and (Bottom) Dose response of functional siCD45 knockdown with Ab-LNP formulations.

### Example 7: In Vivo RNA Delivery to Murine Bone Marrow HSPC Using Various Antibodies

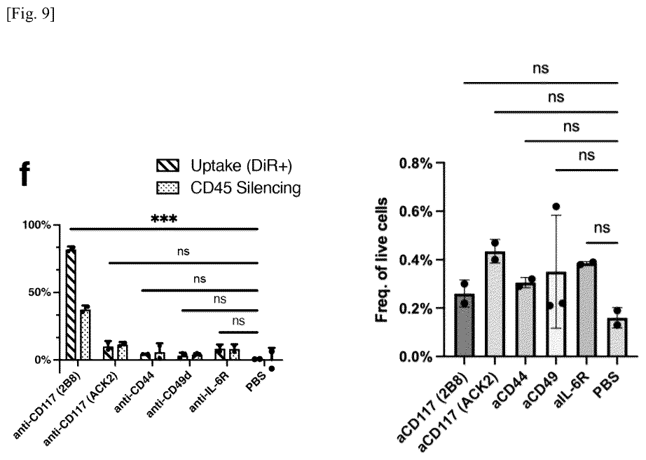

We also wanted to investigate the use of other antibodies that could potentially be used for in vivo delivery to HSPCs. We chose a small panel of other receptors that are expressed on HSPCs (CD49d, CD44, and IL-6R) and conjugated LNPs with antibodies to those receptors. In addition, we also investigated another clone of CD117 (clone ACK2). Among the targets screened, only CD117 was effective for LNP uptake and RNA delivery (FIG. 9, Left). The other antibodies might not be suitable for targeted delivery to HSPCs due to factors such as the clone of the antibody used or receptor dependent factors such as expression level, internalization rate, or off-target tissue expression levels that could serve as antigen sinks. Interestingly, CD117 demonstrated a clonal difference in the performance of Ab-LNPs indicating that the choice of antibody against a specific cell target greatly affects its utility for targeted delivery using antibody decorated lipid nanoparticles. Clone 2B8 is a non-antagonistic clone while clone ACK2 is reported to be antagonistic; however, no depletion of bone marrow HSPCs was observed following Ab-LNP administration with either clone or with Ab-LNPs conjugated to any of the other antibodies (FIG. 9, Right).

### Example 8: In Vitro RNA Delivery to Human Primary HSPC Using an Non-Antagonistic Antibody

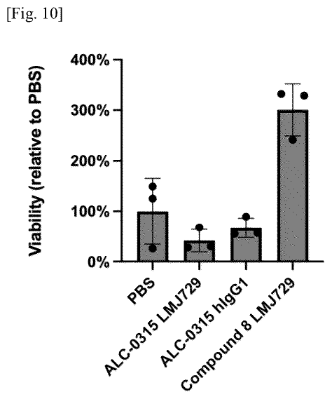

We formulated LNPs with anti-human CD117 antibody clone LMJ729, which is a non-antagonistic antibody, and assessed cellular viability using human primary bone marrow CD34+ cells in vitro. Table 8 and FIG. 10

TABLE 8
LNP formulatins encapsulating Cas9 mRNA and sgRNA against B2M gene

| | | | | | | | Relative |
|---|---|---|---|---|---|---|---|
| Ionizable | | | PEG- | PEG- | Composition | | Viability |
| lipid | Phospholipid | Cholesterol | lipid 1 | lipid 2 | (mol %) | Antibody | (%) |
| ALC-0315 | DSPC | Cholesterol | DSG- | DSPE- | 50:10:38:1.5:0.5 | LMJ729 | 30 |
| | | | PEG2000 | PEG2000- | | | |
| | | | | maleimide | | | |
| ALC-0315 | DSPC | Cholesterol | DSG- | DSPE- | 50:10:38:1.5:0.5 | hIgG1 | 67 |
| | | | PEG2000 | PEG2000- | | | |
| | | | | maleimide | | | |
| Compound | DSPC | Cholesterol | DSG- | DSPE- | 50:10:38:1.5:0.5 | LMJ729 | 300 |
| 8 | | | PEG2000 | PEG2000- | | | |
| | | | | maleimide | | | |

### Example 9: In Vitro RNA Delivery to Human Primary HSPC Using Various Receptor-Antibody Combination

To investigate whether other antibodies could potentially be used for HSPCs delivery, another panel of antibodies was tested. Firefly luciferase mRNA was encapsulated into LNPs that were conjugated to either anti-human CD117 clone 104D2 (non-antagonistic), anti-human CD184 (CXCR4) clone 12G5, anti-human CD105 (Endoglin) clone 43A3, anti-human CD34 clone 581, or their isotype controls as non-targeted controls. Transfection efficiency of these LNPs were tested in vitro using human primary bone marrow CD34+ HSPCs by quantifying luminescence value using Steady-Glo™M Luciferase Assay System (Promega).

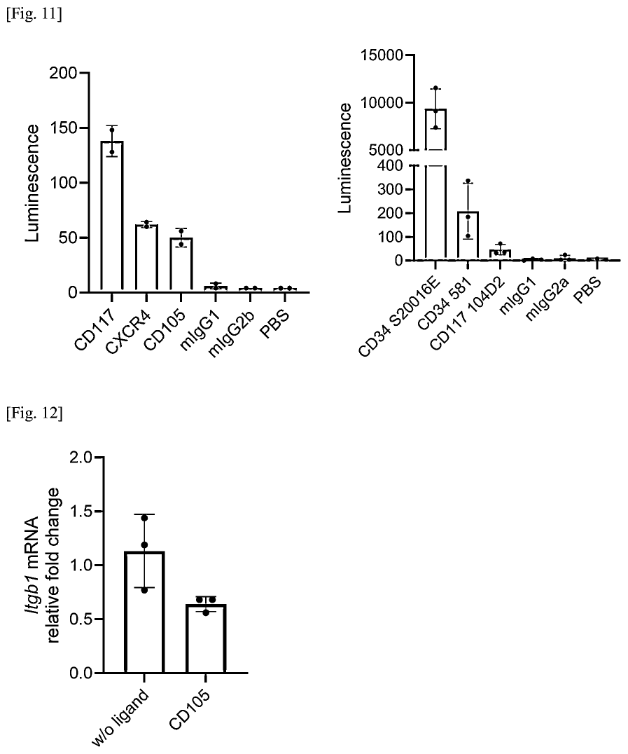

As shown in FIG. 11, all tested targeted LNP formulations showed luciferase expression in vitro at a dose of 100 ng mRNA per 5,000 cells while LNPs conjugated to the isotype control were not effective at transfecting these cells.

TABLE 9
LNP formulatins encapsulating firefly luciferase mRNA

| | Ionizable | Phospho- | | PEG- | PEG- | Composition | |
|---|---|---|---|---|---|---|---|
| | lipid | Lipid | Sterol | lipid 1 | lipid 2 | (mol %) | Antibody |
| | Compound | DSPC | Cholesterol | DMG- | DSPE- | 50:10:38:1.5:0.5 | Anti- |
| | 8 | | | PEG2000 | PEG2000- | | human |
| | | | | | maleimide | | CD117 |
| | | | | | | | mouse |
| | | | | | | | IgG1 |
| | | | | | | | clone |
| | | | | | | | 104D2 |
| | Compound | DSPC | Cholesterol | DMG- | DSPE- | 50:10:38:1.5:0.5 | Anti- |
| | 8 | | | PEG2000 | PEG2000- | | human |
| | | | | | maleimide | | CD184 |
| | | | | | | | mouse |
| | | | | | | | IgG2a |
| | | | | | | | (CXCR4) |
| | | | | | | | clone |
| | | | | | | | 12G5 |
| | Compound | DSPC | Cholesterol | DMG- | DSPE- | 50:10:38:1.5:0.5 | Anti- |
| | 8 | | | PEG2000 | PEG2000- | | human |
| | | | | | maleimide | | CD105 |
| | | | | | | | mouse |
| | | | | | | | IgG1 |
| | | | | | | | clone |
| | | | | | | | 43A3 |
| | Compound | DSPC | Cholesterol | DMG- | DSPE- | 50:10:38:1.5:0.5 | Anti- |
| | 8 | | | PEG2000 | PEG2000- | | human |
| | | | | | maleimide | | CD34 |
| | | | | | | | mouse |
| | | | | | | | IgG2a |
| | | | | | | | Clone: |
| | | | | | | | S20016E |
| | Compound | DSPC | Cholesterol | DMG- | DSPE- | 50:10:38:1.5:0.5 | Anti- |
| | 8 | | | PEG2000 | PEG2000- | | human |
| | | | | | maleimide | | CD34 |
| | | | | | | | mouse |
| | | | | | | | IgG1 |
| | | | | | | | Clone |
| | | | | | | | 581 |
| Comparative | Compound | DSPC | Cholesterol | DMG- | DSPE- | 50:10:38:1.5:0.5 | Mouse |
| example | 8 | | | PEG2000 | PEG2000- | | IgG1 |
| | | | | | maleimide | | Isotype |
| | | | | | | | control |
| | | | | | | | (non- |
| | | | | | | | targeted) |
| Comparative | Compound | DSPC | Cholesterol | DMG- | DSPE- | 50:10:38:1.5:0.5 | Mouse |
| Example | 8 | | | PEG2000 | PEG2000- | | IgG2a |
| | | | | | maleimide | | Isotype |
| | | | | | | | control |
| | | | | | | | (non- |
| | | | | | | | targeted) |

### Example 10: In Vitro RNA Delivery to Mouse Primary Bone Marrow or Mesenchymal Stem Cells (MSC) Using Anti-CD105-LNP

To test our targeted LNPs can be also useful to other stem cells, RNA delivery efficiency of anti-CD105 LNPs was also tested in murine primary bone marrow mesenchymal stem cells (MSCs) in vitro. siRNA against murine integrin β1 (Itgb1) was encapsulated in anti-CD105 antibody-modified LNPs and unmodified LNPs as described in ** Frozen murine primary bone marrow MSCs were obtained from CellBiologics, and cultured in RPMI medium containing 10% FBS. MSCs were then transferred to 96-well plates at a density of 10,000-15,000 cells per well, and culture media was replaced with 90 μl of serum-free RPMI media. MSCs were transfected with 10 μl of LNP solution encapsulating Itgb1 siRNA duplexes at a final concentration of 100 nM. 4 hours post transfection, cell culture media was replaced with serum-containing media. Following incubation of 24 hours, the treated cells were harvested, and the remaining Itgb1 mRNA level was measured in each condition by RT-qPCR. The result is shown in FIG. 12.

TABLE 10
LNP formulatins encapsulating siRNA against Itgb1

| | Ionizable | | | PEG- | PEG- | Composition | |
|---|---|---|---|---|---|---|---|
| | lipid | Phospholipid | Cholesterol | lipid 1 | lipid 2 | (mol %) | Antibody |
| | Compound | DSPC | Cholesterol | DMG- | DSPE- | 50:10:38:1.5:0.5 | Anti- |
| | 2 | | | PEG2000 | PEG2000- | | murine |
| | | | | | maleimide | | CD105 |
| | | | | | | | clone |
| | | | | | | | MJ7/18 |
| Comparative | Compound | DSPC | Cholesterol | DMG- | — | 50:10:38.5:1.5:0 | — |
| example | 2 | | | PEG2000 | | | |

### Example 11: In Vitro RNA Delivery to Human Primary HSPC Using Various Ionizable Lipids (2)

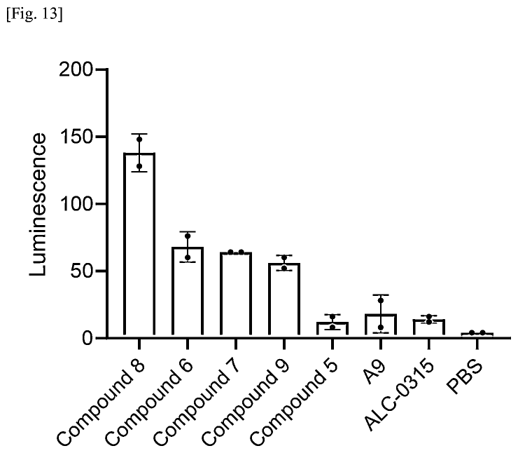

We formulated targeted Ab-LNPs with various ionizable lipids with firefly luciferase mRNA and assessed luminescence 24 h after transfection using Steady-Glo™ Luciferase Assay System (Promega). As shown in FIG. 13, all tested formulations showed luminescence higher than PBS control at a dose of 100 ng mRNA per 5,000 cells.

TABLE 11
LNP formulatins encapsulating firefly luciferase mRNA

| Ionizable | | | PEG- | PEG- | Composition | |
|---|---|---|---|---|---|---|
| lipid | Phospholipid | Cholesterol | lipid 1 | lipid 2 | (mol %) | Antibody |
| ALC-0315 | DSPC | Cholesterol | DMG- | DSPE- | 50:10:38:1.5:0.5 | Anti-human |
| | | | PEG2000 | PEG2000- | | CD117 |
| | | | | maleimide | | 104D2 |
| A9 | DSPC | Cholesterol | DMG- | DSPE- | 50:10:38:1.5:0.5 | Anti-human |
| | | | PEG2000 | PEG2000- | | CD117 |
| | | | | maleimide | | 104D2 |
| Compound | DSPC | Cholesterol | DMG- | DSPE- | 50:10:38:1.5:0.5 | Anti-human |
| 5 | | | PEG2000 | PEG2000- | | CD117 |
| | | | | maleimide | | 104D2 |
| Compound | DSPC | Cholesterol | DMG- | DSPE- | 50:10:38:1.5:0.5 | Anti-human |
| 6 | | | PEG2000 | PEG2000- | | CD117 |
| | | | | maleimide | | 104D2 |
| Compound | DSPC | Cholesterol | DMG- | DSPE- | 50:10:38:1.5:0.5 | Anti-human |
| 7 | | | PEG2000 | PEG2000- | | CD117 |
| | | | | maleimide | | 104D2 |
| Compound | DSPC | Cholesterol | DMG- | DSPE- | 50:10:38:1.5:0.5 | Anti-human |
| 8 | | | PEG2000 | PEG2000- | | CD117 |
| | | | | maleimide | | 104D2 |
| Compound | DSPC | Cholesterol | DMG- | DSPE- | 50:10:38:1.5:0.5 | Anti-human |
| 9 | | | PEG2000 | PEG2000- | | CD117 |
| | | | | maleimide | | 104D2 |

### Example 12: In Vivo Cre Recombinase-Mediated Gene Editing of Bone Marrow HSPC Leads to Long-Term Myeloid and Lymphoid Genetic Conversion

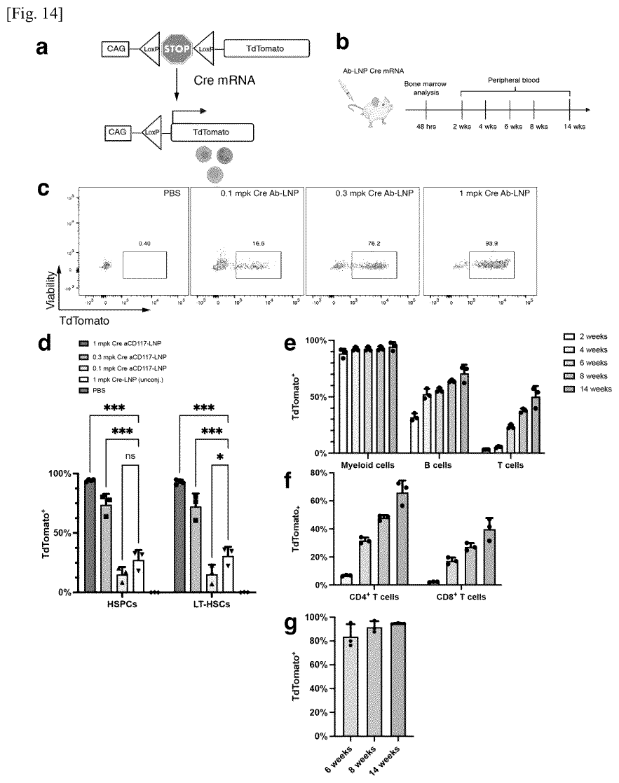

Next we transitioned to evaluating the delivery efficacy of anti-CD117 LNPs with mRNA. For this, we utilized the transgenic Ai14 mouse model, which contains a LoxP-flanked stop cassette that prevents the transcription of fluorescent protein TdTomato. Upon Cre recombination (via delivery of Cre mRNA), the stop cassette is excised out, and the cell then constitutively expresses TdTomato (FIG. 14a). The level of gene editing in HSPCs following treatment with our Ab-LNP was then determined using flow cytometry. In addition, after LNP administration, we tracked the mature immune cells in the peripheral blood long-term to see the level of TdTomato expression in edited progeny at 2, 4, 6, 8, and 14 weeks (FIG. 14b).

We first performed a dose response of anti-CD117 LNP (anti-mouse CD117, clone 2B8, ALC-0315) at doses of 0.1, 0.3, and 1 mg kg-1 Cre mRNA. At a single administration with a dose of 0.3 mg kg-1 (6 μg) mRNA, 48 h after administration, we observed highly efficient delivery with about 75% TdTomato+ cells in both the HSPC and LT-HSC populations. When the dose was increased to 20 μg of mRNA, almost all (−90%) of the HSPCs and LT-HSCs was transfected (FIG. 14d). Unconjugated LNPs at a dose of 1 mg kg-1 Cre mRNA showed about 25% TdTomato expression, indicating that conventional untargeted LNP formulations have the capability of transfecting HSPCs at a low level. The level of delivery is greatly enhanced by the incorporation of an HSPC targeting ligand. Stemness of the transfected HSPCs was maintained in both short-term and long-term as shown by analysis of the peripheral blood populations (erythrocytes, myeloid cells, B cells, and T cells). At 2 weeks, CD11b+ myeloid cells, which consist of granulocytes and monocytes, were already 90% TdTomato+. Since B and T cells are cells with longer lifespans, the level of TdTomato expression for those cell types naturally lagged behind that of myeloid cells at 2 weeks (−32% for B cells and −3.3% for T cells), which increased to −70% TdTomato+ B cells and −50% TdTomato+ T cells by 14 weeks (FIG. 14e). Analysis of T cell subsets (CD4 and CD8) was performed at 4 weeks after LNP administration when the level of TdTomato expression in T cells became more apparent. At each time point, there was a higher population of TdTomato cells in CD4 T cells compared with that in CD8 T cells (FIG. 14f). Erythrocytes also showed almost 100% TdTomato expression at 14 weeks (FIG. 14g). Overall, almost all the HSPCs were transfected, which then resulted in high levels of corrected progeny in all analyzed immune cell populations.

### Example 13: PEG Lipid

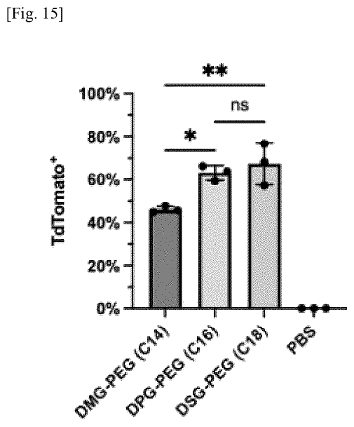

Ab-LNP formulations containing Cre mRNA and different PEG-lipids were injected to Ai14 mice at a dose of 0.3 mg kg⁻¹. 48 hours after injection, mice were sacrificed, and right hind leg was collected for flow cytometry analysis. TdTomato expression was evaluated in bone marrow LSK cells. The results are shown in FIG. 15. Statistics performed by one-way ANOVA with Tukey's multiple comparison test (*P<0.05, **P<0.01).

### Example 14: Dexamethasone Pretreatment Improved Clinical Signs After LNP Administration

Mice were pretreated with PBS or 9 mg/kg dexamethasone (intraperitoneal injection) 1 hour prior to lipid nanoparticle (LNP) administration. Clinical examination was conducted 6 hours post-LNP administration. The results are shown in Table 13.

TABLE 12
LNP formulatin tested in dexamethasone pretreted mice

| Ionizable | | | PEG- | PEG- | Composition | |
|---|---|---|---|---|---|---|
| lipid | Phospholipid | Cholesterol | lipid 1 | lipid 2 | (mol %) | Antibody |
| ALC- | DSPC | Cholesterol | DSG- | DSPE- | 50:10:38:1.5:0.5 | Anti- |
| 0315 | | | PEG2000 | PEG2000- | | mouse |
| | | | | maleimide | | CD117 |
| | | | | | | Clone |
| | | | | | | 2B8 |

TABLE 13
Clinical examination 6 hours post-LNP administration
(1 mg/kg mRNA encoding firefly luciferase)

| | | | Dexamethasone | Dexamethasone |
|---|---|---|---|---|
| | | | pre-treatment | pre-treatment |
| Clinical sign | Qualifier | Score | (+) | (−) |
| Vocalization | None | 0 | 0 | 0 |
| | Provoked vocalization | 5 | | |
| | Unprovoked vocalization | 10 | | |
| Chromo-dacryorrhea | None | 0 | 0 | 0 |
| | Sligh porphyrin staining | 1 | | |
| | Excessive porphyrin | 10 | | |
| | staining | | | |
| Palpebral aperture | Open | 0 | 0 | |
| | Half closed | 4 | | 4 |
| | Sunken and half closed | 10 | | |
| Hunching | Normal posture | 0 | 0 | |
| | Hunched posture | 1 | | 1 |
| Hypokinesia | Normal activity | 0 | 0 | |
| | Reduced activity | 5 | | 5 |
| | Inactive | 5 | | |
| Piloerection | None | 0 | 0 | |
| | Piloerection | 1 | | 1 |

1. A lipid composition comprising (A) a therapeutic agent and (B) a lipid nanoparticle conjugated to a targeting molecule,
wherein the lipid nanoparticle comprises an ionizable lipid, and
the targeting molecule specifically binds to a marker of hematopoietic stem/progenitor cells or mesenchymal stem cells.

2. The lipid composition of claim 1, wherein the lipid nanoparticle comprises a PEG-lipid conjugated to the targeting molecule

3. The lipid composition of claim 1, wherein the ionizable lipid has at least one ionizable amino group and at least one biodegradable group, and wherein the biodegradable group is represented by —O(CO)O—, —O(CO)—, —(CO)O— or S—S.

4. The lipid composition of claim 1, wherein the ionizable lipid is a compound represented by formula (4): 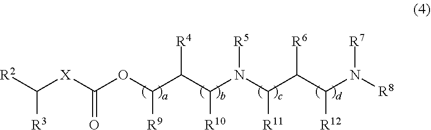
wherein X represents NR¹— or —O—,
R¹ represents a hydrogen atom, a hydrocarbon group having 6 to 24 carbon atoms, or a group represented by R²¹-L¹-R²²—, R²¹ represents a hydrocarbon group having 1 to 24 carbon atoms, and L¹ represents —O(CO)O—, —O(CO)—, —(CO)O—, —O—, or 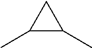
R²² is a divalent linking group and represents a hydrocarbon linking group having 1 to 18 carbon atoms,
R² and R³ each independently represent a hydrogen atom, a hydrocarbon group having 3 to 24 carbon atoms, or a group represented by R³¹-L²-R³²—, R³¹ represents a hydrocarbon group having 1 to 24 carbon atoms, and L² represents —O(CO)O—, —O(CO)—, —(CO)O—, —O—, or 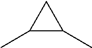
R³² is a divalent linking group and represents a hydrocarbon linking group having 1 to 18 carbon atoms,
each of R⁴, R⁵, R⁶, R⁷, R⁸, R⁹, R¹⁰, R¹¹, and R¹² independently represents a hydrogen atom or an optionally substituted alkyl group having 1-18 carbon atoms,
any one or more sets of R⁴ and R⁵, R¹⁰ and R⁵, R⁵ and R¹², R⁴ and R⁶, R⁵ and R⁶, R⁶ and R⁷, R⁶ and R¹⁰, R¹² and R⁷, and R⁷ and R⁸, may be linked together to form a 4- to 7-membered ring which may contain O atom,
a substituents on the optionally substituted alkyl group having 1-18 carbon atoms represents a hydroxyl groups, a carboxyl groups, an amino groups represented by NR⁴⁵R⁴⁶, a substituted or unsubstituted aryl group, a substituted or unsubstituted heteroaryl group, or a group represented by —O(CO)O—R⁴¹, —O(CO)—R⁴², —(CO)O—R⁴³, or —O—R⁴⁴, wherein R⁴¹, R⁴², R⁴³, R⁴⁴, R⁴⁵ and R⁴⁶ each independently represents a hydrocarbon group having 1 to 18 carbon atoms,
a substituents on the substituted or unsubstituted aryl group and the substituted or unsubstituted heteroaryl group represents an alkyl groups having 1 to 18 carbon atoms, a hydroxyl groups, a carboxyl groups, an amino groups represented by —NR⁴⁵R⁴⁶, or a group represented by —O(CO)O—R⁴¹, —O(CO)—R⁴², —(CO)O—R⁴³, or —O—R⁴⁴, and R⁴¹, R⁴², R⁴³, R⁴⁴, R⁴⁵ and R⁴⁶ each independently represents a hydrocarbon group having 1 to 18 carbon atoms,
a, b, c, and d each independently represents an integer from 0 to 3, wherein a+b is 1 or more, and c+d is 1 or more.

5. The lipid composition of claim 1, wherein the ionizable lipid is a compound represented by formula (1): 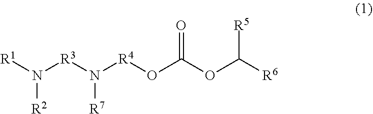
In the formula,
R¹ and R² each independently represent a hydrocarbon group having 1 to 18 carbon atoms, and R³ represents a hydrocarbon group having 2 to 8 carbon atoms, where the hydrocarbon groups represented by R¹, R², and R³ may be substituted with one or more substituents selected from —OH, COOH, —NR⁵¹R⁵², —OC(O)O—R⁵³, —C(O)O—R⁵⁴, —OC(O)—R⁵⁵, and —O—R⁵⁶,
R⁴ represents a hydrocarbon group having 1 to 8 carbon atoms,
R⁵ and R⁶ each independently represent a hydrocarbon group having 1 to 8 carbon atoms or —R⁸-L¹-R⁹, excluding a case that both R⁵ and R⁶ are hydrocarbon groups having 1 to 8 carbon atoms,
R⁷ represents —R¹⁰-L²-R¹¹-L³-R¹²,
R⁵¹ and R⁵² each independently represent a hydrocarbon group having 1 to 8 carbon atoms,
R⁵³, R⁵⁴, R⁵⁵, and R⁵⁶ each independently represent a hydrocarbon group having 1 to 24 carbon atoms,
the hydrocarbon groups represented by R⁵³, R⁵⁴, R⁵⁵, and R⁵⁶ may be substituted with an aryl group having 6 to 20 carbon atoms or —S—R⁵⁸,
the above-described aryl group having 6 to 20 carbon atoms may be substituted with —OH, COOH, —NR⁵¹R⁵², —OC(O)O—R⁵³, —C(O)O—R⁵⁴, —OC(O)—R⁵⁵, —O—R⁵⁶, or -(hydrocarbon group having 1 to 12 carbon atoms)-R⁵⁷,
R⁵⁸ represents a hydrocarbon group having 1 to 12 carbon atoms, and
R⁵⁷ represents —OH, COOH, —NR⁶¹R⁶², —OC(O)O—R⁶³, —C(O)O—R⁶⁴, —OC(O)—R⁶⁵, or —O—R⁶⁶.
R⁶¹ and R⁶² each independently represent a hydrocarbon group having 1 to 8 carbon atoms,
R⁶³, R⁶⁴, R⁶⁵, and R⁶⁶ each independently represent a hydrocarbon group having 1 to 24 carbon atoms,
the hydrocarbon groups represented by R⁶³, R⁶⁴, R⁶⁵, and R⁶⁶ may be substituted with an aryl group having 6 to 20 carbon atoms or —S—R⁶⁸,
the above-described aryl group having 6 to 20 carbon atoms may be substituted with —OH, COOH, —NR⁶¹R⁶², —OC(O)O—R⁶³, —C(O)O—R⁶⁴, —OC(O)—R⁶⁵, —O—R⁶⁶, or -(hydrocarbon group having 1 to 12 carbon atoms)-R⁶⁷,
R⁶⁸ represents a hydrocarbon group having 1 to 12 carbon atoms, and
L¹, L², and L³ each independently represent —OC(O)O—, —C(O)O—, —OC(O)—, or —O—.
R⁸ represents a hydrocarbon group having 1 to 12 carbon atoms,
R⁹ represents a hydrocarbon group having 1 to 24 carbon atoms,
R¹⁰ represents a hydrocarbon group having 1 to 8 carbon atoms,
R¹¹ represents a hydrocarbon group having 1 to 24 carbon atoms,
R¹² represents a hydrocarbon group having 1 to 24 carbon atoms,
the hydrocarbon groups represented by R⁹ and R¹² may be substituted with an aryl group, —OC(O)O—R⁵³, —C(O)O—R⁵⁴, —OC(O)—R⁵⁵, or —S—R⁵⁸, where definitions of R⁵³, R⁵⁴, R⁵⁵, and R⁵⁸ are as described above, and
the hydrocarbon group represented by R¹¹ may be substituted with —OC(O)O—R⁵³, —C(O)O—R⁵⁴, or —OC(O)—R⁵⁵, where the definitions of R⁵³, R⁵⁴, and R⁵⁵ are as described above.

6. The lipid composition of claim 1, wherein the ionizable lipid is a compound represented by the following formula (5): 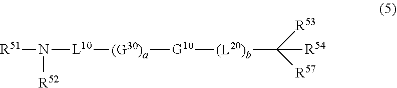
wherein R⁵¹ and R⁵² each independently represent a hydrocarbon group having 1 to 21 carbon atoms which may have a substituent A,
the substituent A represents a hydroxyl group, or a group represented by -G²⁰-CH(R⁵⁵)(R⁵⁶), —N(R⁵⁸)(R⁵⁹) or -G²⁰-R⁶⁰,
G²⁰ represents —O(CO)—, or —(CO)O—,
R⁵⁵ and R⁵⁶ each independently represent a hydrogen atom or a hydrocarbon group having 1 to 18 carbon atoms,
R⁵⁸ and R⁵⁹ each independently represent a hydrogen atom or a cyclic hydrocarbon group having 3 to 6 carbon atoms which may have a substituent B,
the substituent B is —N(R⁶¹)(R⁶²),
R⁶¹ and R⁶² each independently represent a hydrogen atom or a hydrocarbon group having 1 to 6 carbon atoms,
R⁶⁰ represents a hydrocarbon group having 1 to 18 carbon atoms,
L¹⁰ represents a hydrocarbon group having 1 to 18 carbon atoms,
G³⁰ indicates —S—(CO)—NR⁶⁴,
R⁶⁴ represents a group represented by -L³⁰-G²⁰-CH(R⁵⁵)(R⁵⁶),
a represents 0 or 1,
L³⁰ represents a single bond or a hydrocarbon group having 1 to 18 carbon atoms,
G¹⁰ represents —O(CO)—, —(CO)O—, —O(CO)O—, or —N(C(O)R⁶³)—,
R⁶³ represents a hydrocarbon group having 1 to 18 carbon atoms,
L²⁰ represents a hydrocarbon group having 1 to 6 carbon atoms,
b represents 0 or 1,
R⁵³, R⁵⁴ and R⁵⁷ each independently represent a hydrogen atom or a hydrocarbon group having 1 to 21 carbon atoms which may have a substituent C,
the substituent C represents a group represented by —(CO)OR⁶⁵ or —O(CO)—R⁶⁵,
R⁶⁵ represents a hydrocarbon group having 1 to 18 carbon atoms or a group represented by -L⁴⁰-CH(R⁶⁶)(R⁶⁷),
L⁴⁰ represents a hydrocarbon group having 1 to 6 carbon atoms,
R⁶⁶ and R⁶⁷ represent a hydrocarbon group having 1 to 10 carbon atoms or an alkoxy group.

7. The lipid composition of claim 1, wherein the ionizable lipid is at least one which is selected from the compounds represented by the following formulas.

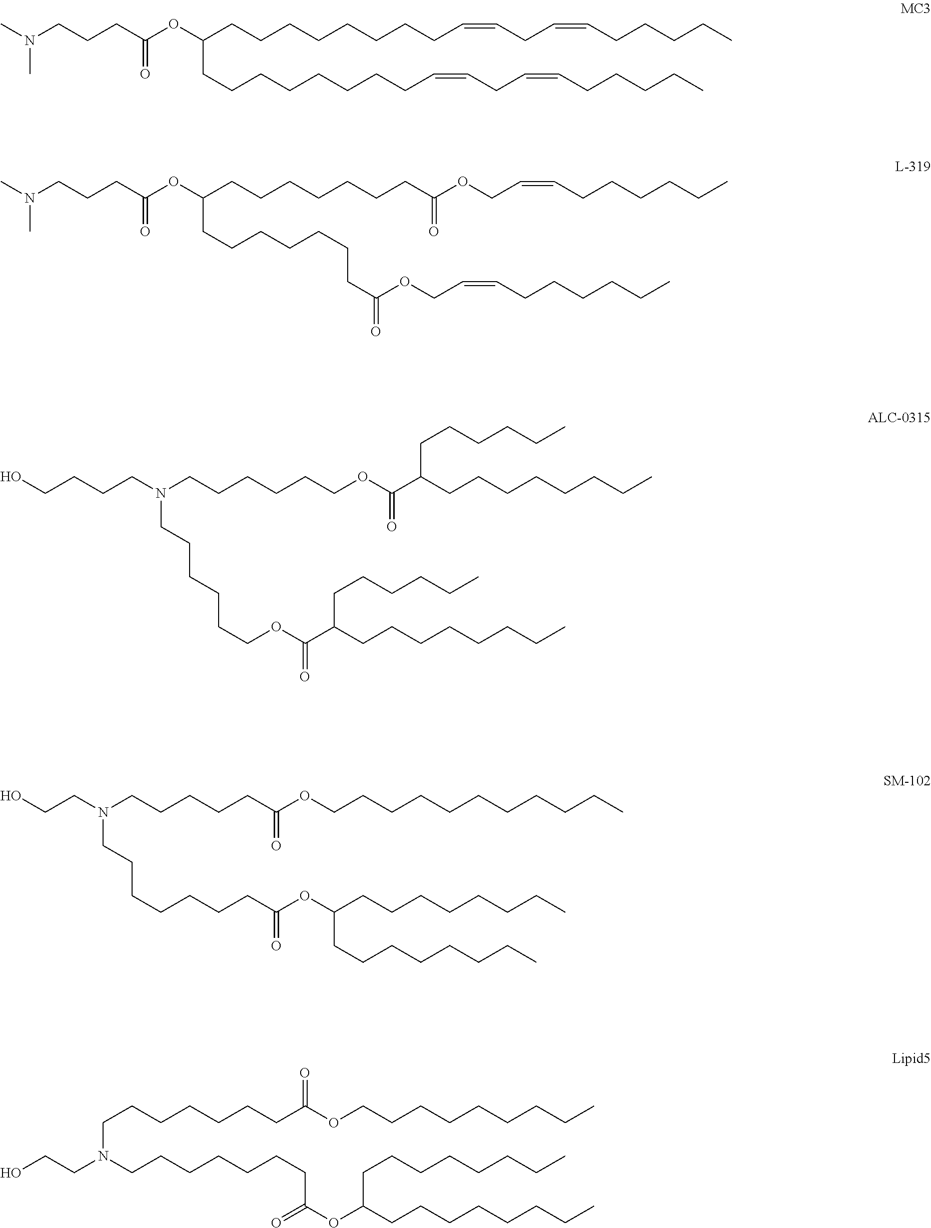

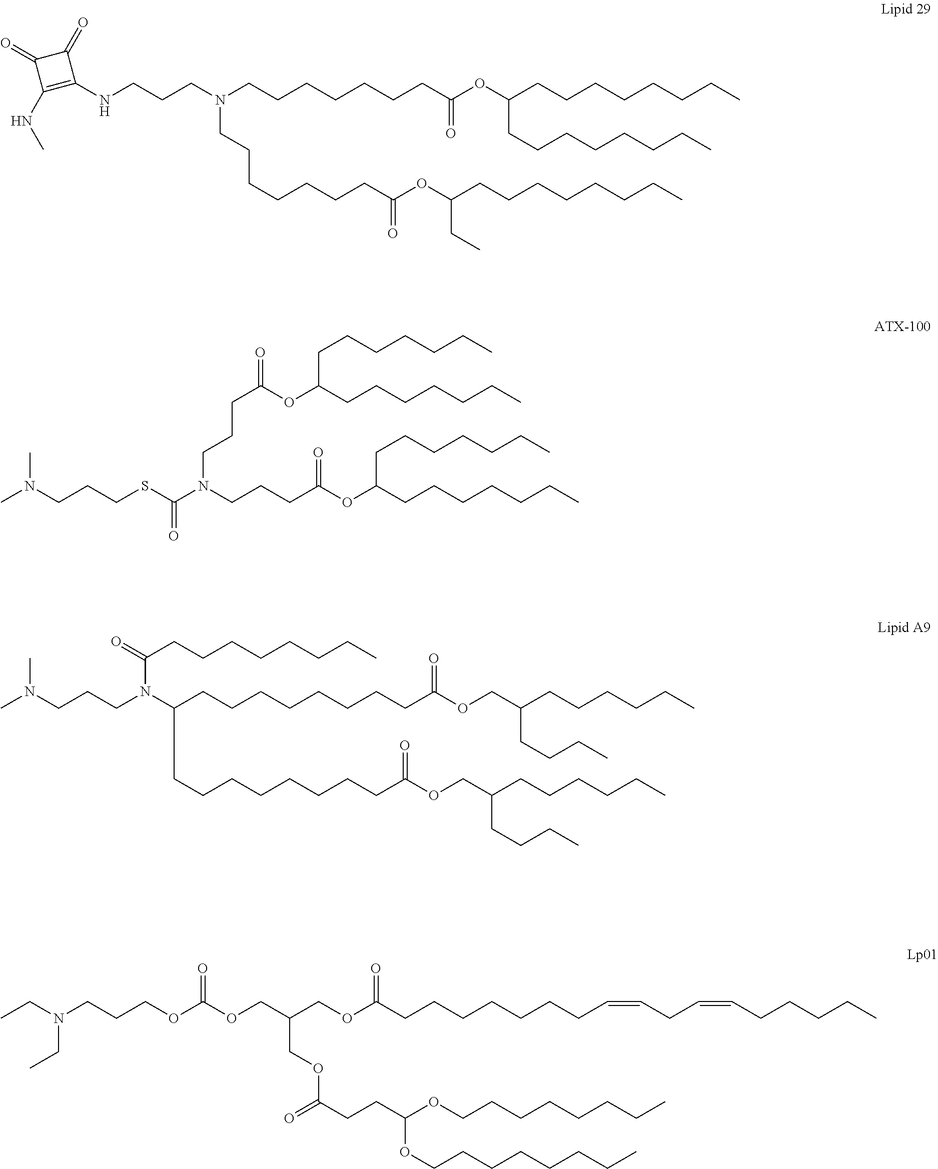

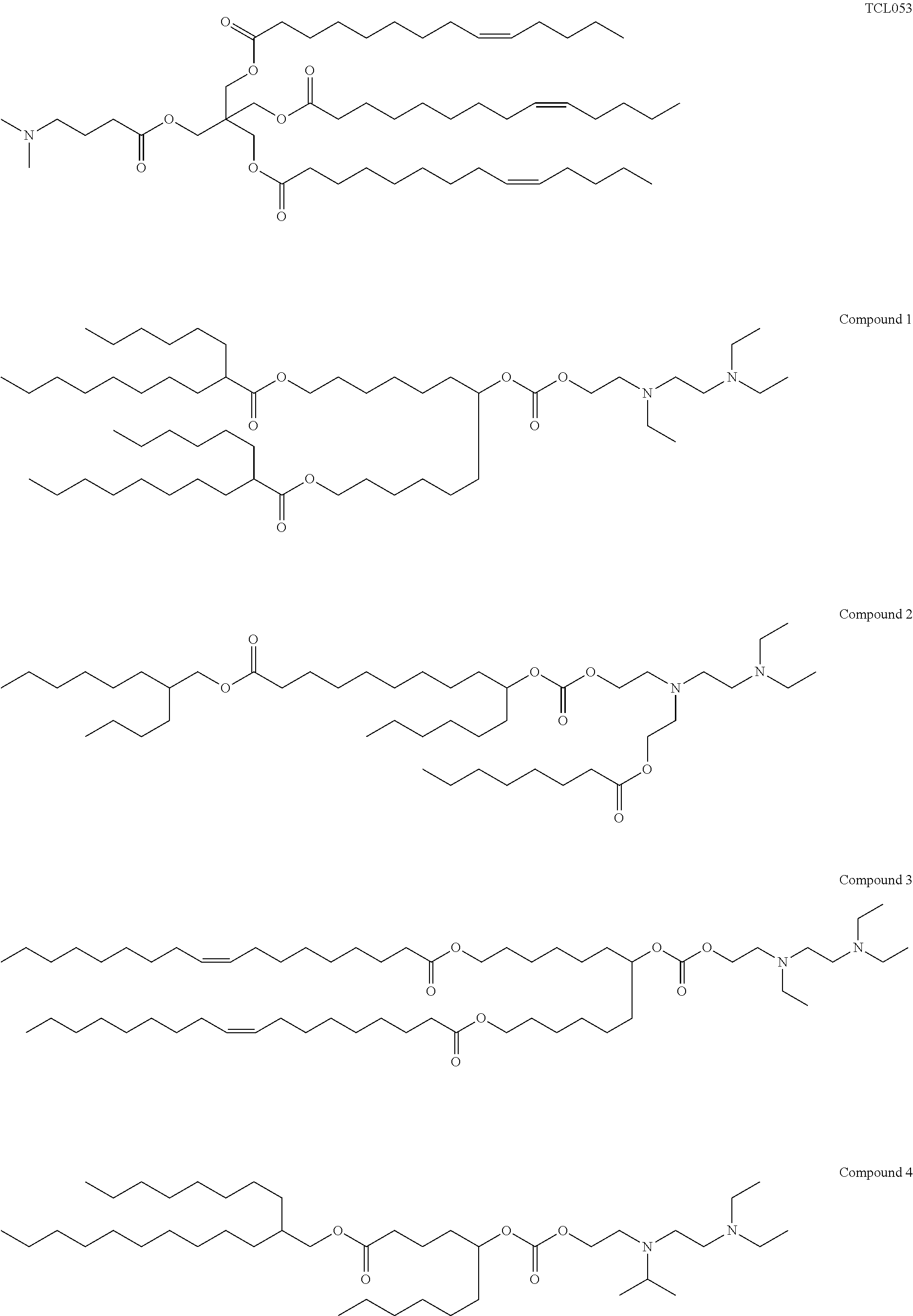

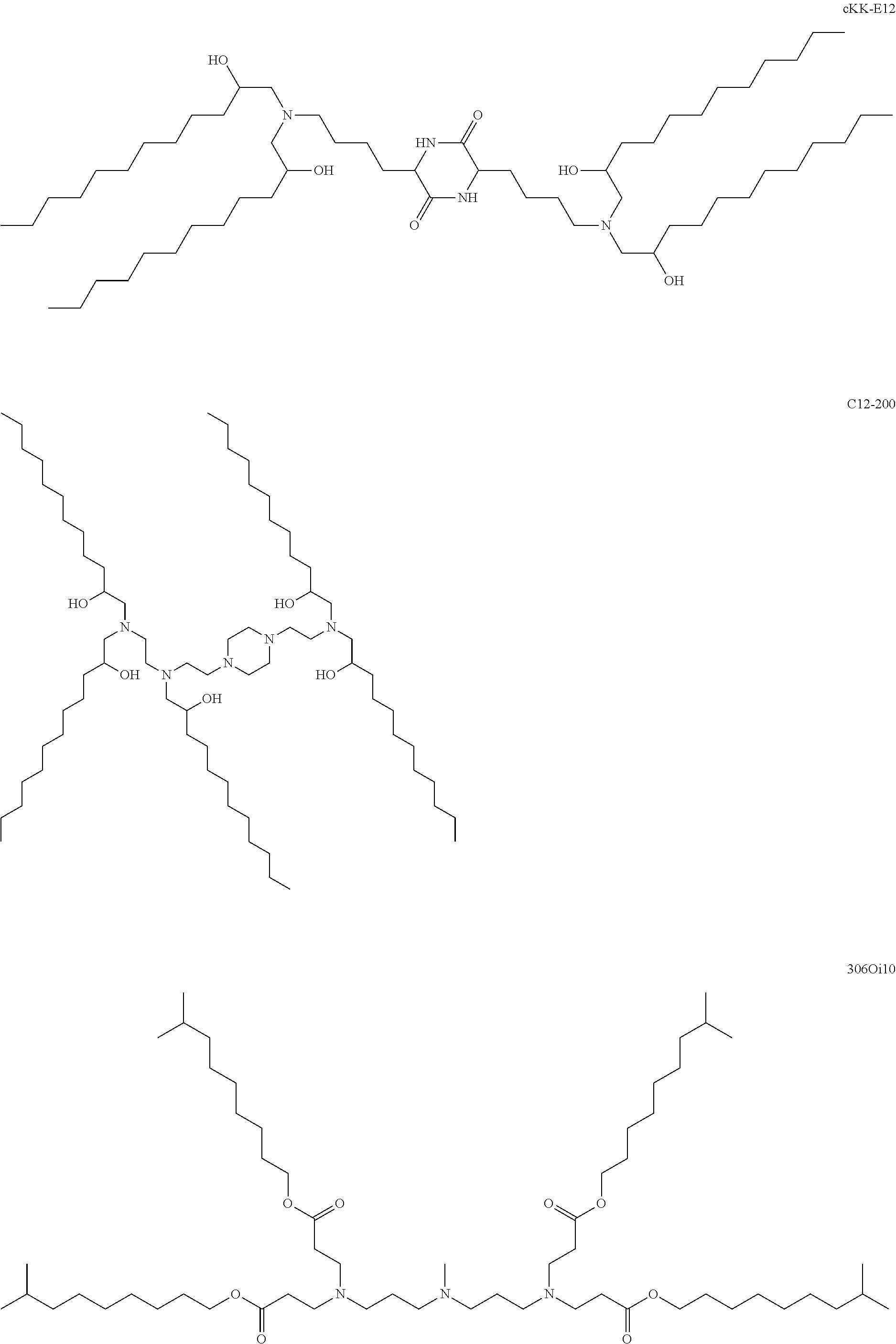

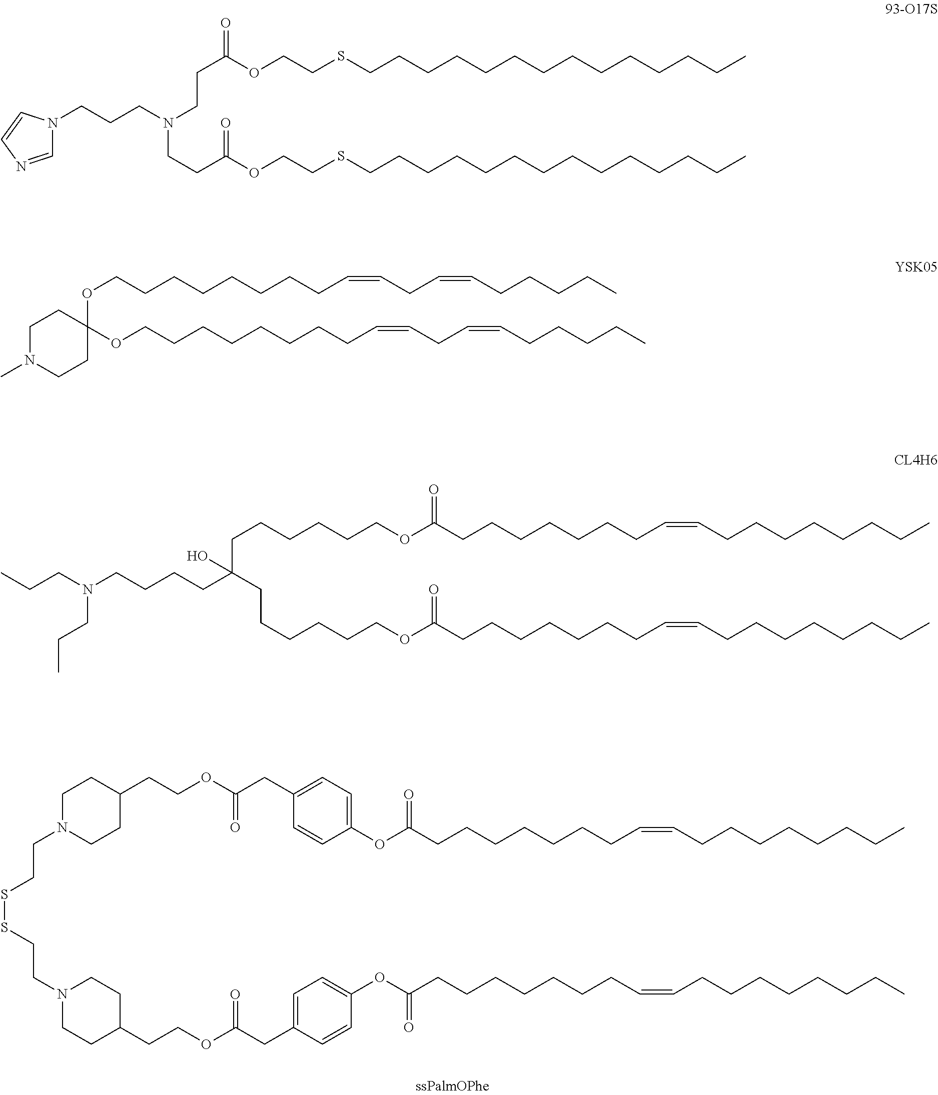

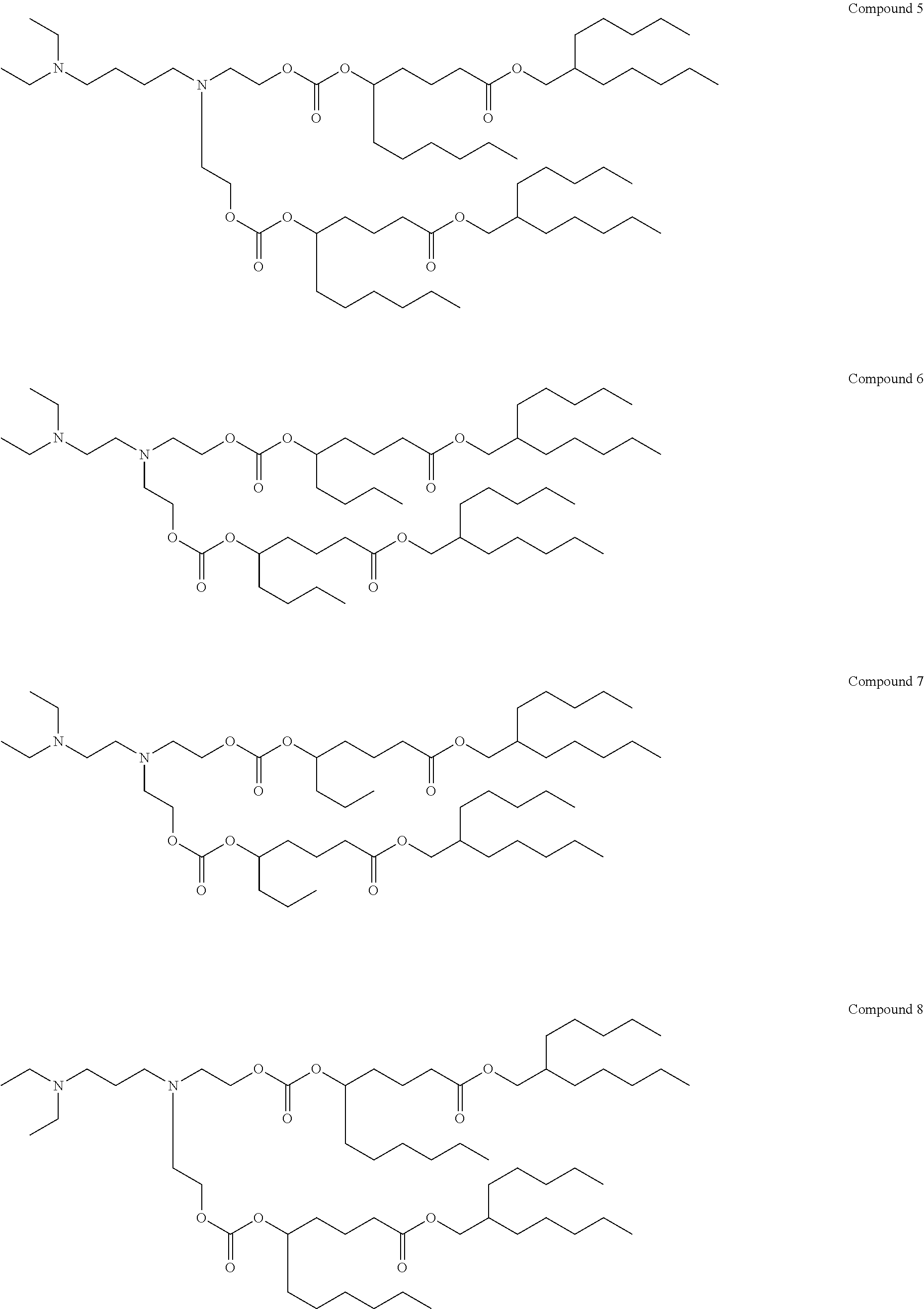

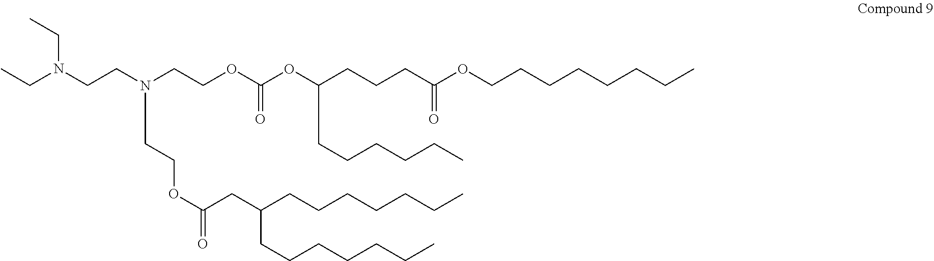 [SMILES: CCCCCCCCOC(=O)CCCC(CCCCCC)OC(=O)OCCN(CCOC(=O)CC(CCCCCCC)CCCCCCC)CCN(CC)CC]

8. The lipid composition of claim 1, wherein the lipid nanoparticle comprises a sterol.

9. The lipid composition of claim 1, wherein the lipid nanoparticle comprises a phospholipid.

10. The lipid composition of claim 1, wherein the therapeutic agent comprises a polynucleotide.

11. The lipid composition of claim 9, wherein the polynucleotide is DNA or RNA.

12. The lipid composition of claim 9, wherein the polynucleotide is mRNA, sgRNA or siRNA.

13. The lipid composition of claim 1, wherein the targeting molecule is at least one which is selected from nucleic acid, peptide, antibody and small molecule.

14. The lipid composition of claim 12, wherein the targeting molecule is antibody.

15. The lipid composition of claim 1, wherein the marker of hematopoietic stem/progenitor cells is CD34, CD105, CD117, or CD184 (CXCR4).

16. The lipid composition of claim 1, wherein the marker of hematopoietic stem/progenitor cells is CD117.

17. The lipid composition of claim 1, wherein the marker of mesenchymal stem cell is CD105.

18. A method for delivering a therapeutic agent to a cell which expresses a marker of hematopoietic stem/progenitor cells or mesenchymal stem cells., which comprises administering the lipid composition of claim 1 to a subject.

19. The method of claim 18, which further comprises administering a therapeutically effective amount of an inflammatory reducing agent to the subject prior to administering a lipid composition comprising (A) a therapeutic agent and (B) a lipid nanoparticle conjugated to a targeting molecule to the subject,
wherein the lipid nanoparticle comprises an ionizable lipid, and
the targeting molecule specifically binds to a marker of hematopoietic stem/progenitor cells or mesenchymal stem cells.

20. The method of claim 19, wherein the inflammatory reducing agents is selected from (a) corticosteroids, (b) antihistamines, (c) acetaminophen, (d) NSAIDS, (e) kinase inhibitors with CD117 kinase activity inhibiting activity, or (f) other immunosuppressants.

21. A method of reducing adverse effects related to anti-CD117 antibody-modified LNP administration, the method comprising administering a therapeutically effective amount of an inflammatory reducing agent to a subject prior to administering CD117 antibody-modified LNP.

22. The method of claim 21, wherein the inflammatory reducing agents is selected from (a) corticosteroids, (b) antihistamines, (c) acetaminophen, (d) NSAIDS, (e) kinase inhibitors with CD117 kinase activity inhibiting activity, or (f) other immunosuppressants.
